(19) United States
(12) Patent Application Publication
(10) Pub. No.: US 2016/0136272 A1
(43) Pub. Date: May 19, 2016

(54) METHODS FOR RESTORING CORTICOSTEROID SENSITIVITY

(71) Applicant: INNATE PHARMA, Marseille (FR)

(72) Inventors: RENAUD BUFFET, BOULOGNE (FR); CARINE PATUREL, MARCY L'ETOILE (FR)

(21) Appl. No.: 14/890,921

(22) PCT Filed: May 13, 2014
(86) PCT No.: PCT/EP2014/059773

§ 371 Date: Nov. 13, 2015

Related U.S. Application Data
(60) Provisional application No. 61/822,997, filed on May 14, 2013.

(51) Int. Cl. A61K 39/395; C07K 16/28; A61K 31/573

(52) U.S. Cl. CPC A61K 39/3955; A61K 31/573; C07K 16/2896; A61K 2039/505

(57) ABSTRACT

The present invention relates to use of therapeutic agents that specifically bind and inhibit TLR3 signalling in order to sensitize patients to treatment with corticosteroids, notably for the treatment and prevention of inflammatory and autoimmune disorders.

### CROSS-REFERENCE TO RELATED APPLICATIONS

This application claims the benefit of U.S. Provisional Application Nos. 61/822,997 filed 14 May 2013; which is incorporated herein by reference in its entirety; including any drawings.

### REFERENCE TO SEQUENCE LISTING

The present application is being filed along with a Sequence Listing in electronic format. The Sequence Listing is provided as a file entitled “PCT Seq list TLR3-6_5 T25”, created May 9, 2014, which is 38 KB in size. The information in the electronic format of the Sequence Listing is incorporated herein by reference in its entirety.

### FIELD OF THE INVENTION

The present invention relates to use of therapeutic agents (e.g. small molecules, antibodies, antibody fragments, and derivatives thereof) that specifically bind TLR3, and that inhibit, signalling, for the treatment and prevention of inflammatory and autoimmune disorders.

### BACKGROUND

Standard therapy for a variety of immune and inflammatory disorders includes administration of corticosteroids, which have the ability to suppress immunologic and inflammatory responses. Corticoids have a broad scope of action, including reduction in various proinflammatory cytokines. A wide body of clinical experience has given rise to current treatment practice which is to administer oral or intravenous corticoids at high doses of at least about 1 mg/kg per day because severe immune and inflammatory disorders are generally not sufficiently controlled below such doses. Treatment for exacerbations can involve even high doses of corticosteroids, for example 250 mg or 500 mg per day of methylprednisolone, for several days. Toxicity generally arises at about 10 mg/kg per day or more of prednisone-equivalent. Complications associated with prolonged and/or high dose steroid usage include musculoskeletal effects (e.g., osteoporosis, myopathy, aseptic necrosis of bone), ophthalmic effects (e.g., posterior subcapsular cataracts), gastrointestinal effects (e.g., ulcers, pancreatitis, nausea, vomiting), weight gain, induced diabetes, cardiovascular effects (e.g., hypertension, atherosclerosis), central nervous system effects (e.g., pseudotumor cerebri, psychiatric reactions), dermatological effects (e.g., hirsutism, redistribution of subcutaneous fat, impaired wound healing, thinning of the skin), an increase of the increase of the infectious risk, growth retardation in children and suppression of the hypothalamus-pituitary-adrenal axis. Many of the side effects of corticosteroid usage appear to be dose-dependent (Kimberly, R. P. (1992) Curr. Opin. Rheumatol. 4:325-331). Another problem of high dosages of corticosteroids is the occurrence of a “steroid rebound effect” when corticosteroid administration is discontinued. A steroid rebound effect is characterized by the worsening of the inflammatory condition(s) being treated upon cessation of steroid therapy.

A further problem is that patients can become resistant to the effects of corticosteroids and must be treated with other agents (e.g. cyclophosphamide). Finally, some conditions such as COPD, severe sepsis or septic shock are broadly resistant to corticoid treatment. Corticosteroid insensitivity has serious health, societal, and economic costs. For example, a small percentage of patients with asthma (5-10%) have severe corticosteroid-refractory condition that often fails to respond but these patients account for >50% of the total asthma health care costs.

There is therefore a need to provide improved methods and compositions for therapy of inflammatory diseases.

### SUMMARY OF THE INVENTION

The invention arises from the findings that anti-TLR3 agents that inhibit TLR3 signalling (e.g., small molecules, antibodies, antibody fragments, and derivatives thereof that specifically bind TLR3) can render animals having a corticoid insensitive inflammatory or autoimmune conditions sensitive to a corticosteroid therapy. When administered in combination with anti-TLR3 agents, animals regained sensitivity to corticosteroids. The methods open new avenues for restoring or improving corticosteroid sensitivity, a particularly important features for patients receiving (or having received) high dose corticosteroids (e.g. high dose regimens typically administered orally or by infusion or injection) and who are not or no longer sufficiently responsive, e.g., not or no longer sufficiently responsive to standard doses, low doses, etc. The methods are also valuable for decreasing doses of corticosteroids, e.g. to avoid toxic side effects, and can therefore be used in low-dose corticosteroid regimens.

The synergy observed with anti-TLR3 agents and corticosteroids is particularly strong in settings involving multiple populations of immune cells suggesting that the combination will be particularly effective in pathologies characterized by a strong immune component, notably T and/or B cells, as a factor contributing to disease.

The findings presented herein are important because they permit uses of anti-TLR3 agents to sensitize patients having inflammatory or autoimmune disorders to corticosteroids.

In one aspect, provided is a method of sensitizing a patient to treatment with a corticosteroid, the method comprising administering to the patient an anti-TLR3 agent that inhibits TLR3 signalling. In one embodiment, the patient has an inflammatory or autoimmune disorder.

The anti-TLR3 agents are advantageously used in combination therapy. In one embodiment of the treatment methods, anti-TLR3 agents are administered before, concomitantly with or after a corticosteroid.

In one aspect, the provided is a method of treating a subject (e.g. an individual, a patient), the method comprising administering to the subject an anti-TLR3 agent that inhibits TLR3 signalling, in combination with a corticosteroid. In one embodiment, the subject has an inflammatory or autoimmune disorder. In one aspect, provided is the use of an anti-TLR3 agent that inhibits TLR3 signalling, in combination with a corticosteroid, for the treatment of an inflammatory or autoimmune disease, e.g. a chronic inflammatory or autoimmune disease. In one embodiment of any of the methods or uses, the corticosteroid is administered orally or by injection. In one embodiment of any of the methods or uses, the corticosteroid is administered at a dose of at least 1 mg/kg per day of prednisone equivalent (a dose of a corticosteroid that is equivalent to a dose of at least 1 mg/kg per day of prednisone). Equivalent doses between corticosteroids are known in the art and can be determined according to conversion guidelines. For example, 5 mg prednisone or prednisolone is equivalent to 20 mg hydroxycortisone, 4 mg methylprednisone or 0.75 mg dexamethasone. Thus, a dose of hydroxycortisone of at least 4 mg/kg per day will correspond to a dose of at least 1 mg/kg per day of prednisone equivalent. In one embodiment of any of the methods or uses, the corticosteroid is administered orally or per injection or infusion, at a dose of at least 1 mg/kg per day, or between 1 mg/kg per day and 10 mg/kg per day of prednisone equivalent. In one embodiment, the treatment comprises the anti-TLR3 agent and the corticosteroid, and one or more additional therapeutic agents used for the treatment of the particularly inflammatory or autoimmune disease (e.g. the treatment is a 3-agent combination therapy, for example anti-TLR3 agent, corticosteroid and a long-acting beta-adrenoceptor agonist in asthma or chronic obstructive pulmonary disease (COPD)). In one embodiment, the treatment comprises the anti-TLR3 agent and the corticosteroid but does not comprise an additional therapeutic agent used for the treatment of the particularly inflammatory or autoimmune disease (e.g. the therapeutic agents used to treat the particular condition is a 2-agent combination therapy).

In one embodiment of any of the methods or uses, the corticosteroid is administered as a low-dose regimen. In one embodiment of any of the methods or uses, the corticosteroid is administered at a dose of less than 10 mg/kg per day of prednisone equivalent. In one embodiment of any of the methods or uses, the corticosteroid is administered at a dose of 1 mg/kg per day or less than 1 mg/kg per day of prednisone equivalent. In one embodiment, of any of the methods or uses, the corticosteroid is administered orally or by injection, at a dose of 1 mg/kg per day or less than 1 mg/kg per day of prednisone equivalent. In one embodiment of any of the methods or uses, the corticosteroid is administered by inhalation at a dose equal to or lower than 1200 μg/day of beclomethasone equivalent (a dose of a corticosteroid that is equivalent to a dose equal to or lower than 1200 μg/day of beclomethasone). In one embodiment of any of the methods or uses of the herein, the corticosteroid is administered by inhalation at a dose higher than 1200 μg/day of beclomethasone equivalent.

In one embodiment of any of the methods or uses, the corticosteroid is administered at a dose of 10 mg per day of prednisone equivalent or less, optionally less than 10 mg of prednisone equivalent per day.

Optionally, in any of the embodiments herein, the corticosteroid is administered daily or once every other day (e.g. at least 3 times per week). In one embodiment, the corticosteroid is administered at less than 10 mg/kg per day of prednisone equivalent. In one embodiment, the corticosteroid used in combination with anti-TLR3 agent is given in a dosage that is at least 25% less than the same corticosteroid administered alone to achieve said therapeutic effect. In one embodiment, the corticosteroid used in combination with anti-TLR3 agent is given in a dosage that is at least 50% less than the same corticosteroid administered alone to achieve said therapeutic effect.

In one aspect, provided is a method of treating a subject (e.g. an individual, a patient) having lupus, the method comprising administering to the subject an anti-TLR3 agent that inhibits TLR3 signalling, in combination with a corticosteroid. In one embodiment, the corticosteroid is administered as a pulse therapy, optionally wherein the corticosteroid is methylprednisolone, optionally between 125 mg/day and 1 gram/day. In one embodiment, the corticosteroid is administered as a low-dose regimen. In one embodiment of any of the methods or uses, the corticosteroid is administered at a dose of less than 10 mg/kg per day of prednisone equivalent. In one embodiment of any of the methods or uses, the corticosteroid is administered at a dose of less than 1 mg/kg per day of prednisone equivalent. In one embodiment of any of the methods or uses, the corticosteroid is administered at a dose of 10 mg per day to 1 mg/kg/day of prednisone equivalent. In one embodiment of any of the methods or uses, the corticosteroid is administered at a dose of 10 mg per day or less, optionally less than 10 mg per day, of prednisone equivalent. In one embodiment of any of the methods or uses, the corticosteroid is administered at a dose of less than 30, 40 or 50 mg per day of prednisone equivalent. Optionally, the corticosteroid is administered daily or once every other day (e.g. at least 3 times per week). In one embodiment, the corticosteroid is administered at less than 10 mg/kg per day. In one embodiment, the corticosteroid is selected from prednisone, prednisolone and methylprednisolone and the corticosteroid is administered at a dose of 1 mg/kg per day or less, optionally 10 mg per day or less of prednisone equivalent, by oral administration, or optionally by intra-muscular (IM) injection into the skin for discoid rashes, or direct injection into a joint. In one embodiment, the corticosteroid is administered less than daily, e.g. every other day, no more than four times per week.

In one aspect, provided is a method of treating a subject (e.g. an individual, a patient) having a respiratory disorder (e.g. asthma, chronic asthma, moderate or severe asthma, COPD) or other immune or inflammatory disorder, the method comprising administering to the subject an anti-TLR3 agent that inhibits TLR3 signalling, in combination with a corticosteroid. In one embodiment, the corticosteroid is administered between 0.1 mg/day and 1 gram/day. In one embodiment, the subject is treated with an anti-TLR3 agent in combination with an inhaled corticosteroid, wherein the dose of the corticosteroid is no more than (or less than) 2 mg per day, optionally less than 1 mg per day, optionally between 0.001 mg per day and 2 mg/day of prednisone equivalent. In one embodiment, the subject (e.g. a subject having severe asthma, COPD, corticosteroid-insensitive disease) is treated with an anti-TLR3 agent in combination with a corticosteroid administered orally or by injection, wherein the dose of the corticosteroid is no more than (or is less than) 10 mg/kg per day of prednisone equivalent, optionally less than 1 mg/kg per day, optionally between 1 mg per day and 60 mg/day of prednisone equivalent. Optionally, the corticosteroid is administered daily or once every other day (e.g. at least 3 times per week).

In one aspect, provided is methods for modulating (increasing) responsiveness to a corticosteroid in a subject (or for treating a subject having corticosteroid insensitivity), comprising administering to the subject suffering from a condition normally responsive to corticosteroid therapy: (a) an anti-TLR3 agent, being administered at a dosage and by a route sufficient to inhibit TLR3 signalling; and (b) a corticosteroid, such that responsiveness of the subject to the corticosteroid is modulated (increased) as compared to when a corticosteroid alone is administered to the subject. Optionally, the anti-TLR3 agent and the corticosteroid are administered such that corticosteroid insensitivity in the subject is reversed, as compared to when a corticosteroid alone is administered to the subject. Optionally, the anti-TLR3 agent and the corticosteroid are administered such that corticosteroid sensitivity in the subject is increased, as compared to when a corticosteroid alone is administered to the subject. Optionally, the anti-TLR3 agent and the corticosteroid are administered to the subject according to a schedule that reduces the dosage of the corticosteroid over time and/or the method ameliorates a steroid rebound effect associated with administration of reduced dosages of the corticosteroid.

In one aspect, provided is the use of an anti-TLR3 agent that inhibits TLR3 signalling, in combination with a corticoid, for the treatment of a corticosteroid-insensitive inflammatory or autoimmune disease (or individual having such disease).

In one embodiment, the corticosteroid-resistant inflammatory or autoimmune disease is a disease which is not sufficiently controlled by administration of a corticosteroid. In one embodiment, the corticosteroid-resistant inflammatory or autoimmune disease is a disease which has been previously treated with a corticosteroid and is not sufficiently controlled by administration of a corticosteroid. In one embodiment of the regimen, the corticoid is administered as a low-dose regimen in combination with the anti-TLR3 agent, optionally wherein the corticosteroid is administered at less than 10 mg/kg per day of prednisone equivalent.

In one embodiment, the corticosteroid-resistant inflammatory or autoimmune disease is a disease which is known not to be responsive to administration of a corticoid. In one embodiment, the disease is COPD. In one embodiment, the disease is corticosteroid-resistant asthma or severe asthma. In one aspect, provided is the use of an anti-TLR3 agent that inhibits TLR3 signalling, in combination with a corticoid, for the treatment of COPD. In one embodiment, the corticosteroid is administered at less than 10 mg/kg per day. In one embodiment, the corticosteroid is administered at less than 1 mg/kg, 2 mg/kg or 3 mg/kg per day of prednisone equivalent. In one embodiment, the corticosteroid is administered at 10 mg/kg per day or more of prednisone equivalent.

In one embodiment, the inflammatory or autoimmune disease treated with the anti-TLR3 agent (and corticosteroid) is a respiratory disorder, optionally asthma (e.g., corticosteroid-resistant asthma, severe asthma), optionally COPD, optionally sarcoidosis. In one embodiment, the inflammatory or autoimmune disease treated with the anti-TLR3 agent (and corticosteroid) is a pathology characterized by a strong immune component, for example T and/or B cells, as a factor contributing to disease. In one embodiment the disease in which T cells contribute to disease. In one embodiment the disease in which B cells contribute to disease. In one embodiment, the inflammatory or autoimmune disease involving B cells treated with the anti-TLR3 agent is lupus, e.g. systemic lupus erythematosus. In one embodiment, the inflammatory or autoimmune disease involving B cells treated with the anti-TLR3 agent is rheumatoid arthritis, Sjogren's syndrome, ANCA-associated vasculitis, antiphospholipid syndrome, idiopathic thrombocytopaenia, autoimmune haemolytic anaemia, Guillian-Barre syndrome, peripheral chronic immune polyneuropathy, autoimmune thyroiditis, Type I diabetes, Addison's disease, membranous glomerulonephropathy, Goodpasture's disease, autoimmune gastritis, pernicious anaemia, pemiphigus vulgarus, primary biliary cirrhosis, dermatomyositis-polymyositis, myasthenia gravis, celiac disease, immunoglobulin A nephropathy, Henoch-Schonlein purpura, chronic graft rejection, atopic dermatitis, asthma or allergy.

In one embodiment, the inflammatory or autoimmune disease treated with the anti-TLR3 agent is an inflammatory bowel disorder.

In one embodiment, corticosteroid-resistant asthma is disease that is not adequately controlled (e.g. presence of recurrent symptoms or exacerbations) by standard corticosteroid therapy, e.g. more than 1200 μg/day of inhaled beclomethasone or equivalent corticosteroid per day, with or without use of systemic (e.g. oral) corticosteroid (e.g. prednisolone) therapy. In one embodiment, corticosteroid-resistant disease (e.g. asthma, lupus, etc.) is a disease that is not adequately controlled at a dose of corticosteroid that causes side-effects in the individual.

In one aspect, provided is a method for the treatment of an autoimmune or inflammatory disease in a patient, comprising: (a) determining whether said patient has corticosteroid insensitivity; and (b) if said patient has corticosteroid insensitivity, administering to said patient an effective dose of anti-TLR3 antibody in combination with a corticosteroid. In one embodiment, said corticosteroid insensitivity is corticosteroid resistance or corticosteroid refractory response. In one embodiment, said corticosteroid insensitivity is corticosteroid dependence. In one embodiment, said corticosteroid insensitivity is corticosteroid intolerance.

In one embodiment, the anti-TLR3 antibodies are administered to a patient at a frequency of from about twice per week to about once every 2 months, optionally in an amount of from about 0.01 to about 3 mg/kg, or any other appropriate dosage as described herein. In one embodiment, provided is an article of manufacture comprising:
(a) a container comprising an anti-TLR3 antibody; and
(b) a package insert with instructions for treating a patient (and/or treating an inflammatory or autoimmune disorder), wherein the instructions indicate that a dose of the anti-TLR3 antibody is administered to the patient at a frequency of from about twice per week to about once every 2 months, in combination with a corticosteroid regimen, optionally a low-dose corticosteroid regimen, optionally a corticosteroid dosage described herein, optionally wherein the instructions further indicate the dose of the anti-TLR3 antibody is about 0.01 to about 3 mg/kg.

In one embodiment, provided is a method of treating an individual having an autoimmune or inflammatory disease, comprising administering to the individual an effective amount of an anti-TLR3 antibody and a corticosteroid, wherein the effective amount is between about 0.01 and 20 mg/kg, optionally between about 0.05 and 20 mg/kg, administered to the individual at a frequency of from about once per week to about once every 2 months.

In one aspect of any embodiment herein, an individual treated has an established or chronic autoimmune or inflammatory disease. In one aspect of any embodiment herein, an individual treated suffers from an attack, crisis, flare or exacerbation in an individual having an established or chronic autoimmune or inflammatory disease.

In one embodiment, provided is a method for the treatment of an autoimmune or inflammatory disease in an individual, comprising:

(a) evaluating whether said individual is corticosteroid insensitive (e.g. has a disease or condition that is corticosteroid insensitive);

(b) if said individual is corticosteroid insensitive, administering to said patient an effective dose of an antibody that binds a TLR3 polypeptide, in combination with a corticosteroid.

In one embodiment, provided is a method for determining the suitability of a patient for treatment with an antibody that binds a TLR3 polypeptide, comprising evaluating whether said patient is corticosteroid insensitive.

In one embodiment of any of the methods described herein, evaluating whether a patient is corticosteroid insensitive comprises treating the patient with (e.g. administering to the patient) a corticosteroid (e.g. a short course of treatment with a corticosteroid (at least 5-7 days, optionally wherein the dose is subsequently tapered), a longer course of treatment with a corticosteroid, a low dose corticosteroid regimen, a high dose corticosteroid regimen, etc.), and observing an insufficient therapeutic response. In one embodiment, the individual is being treated with a corticosteroid at the time of the evaluating step, or has undergone treatment with a corticosteroid.

In one embodiment of any of the methods described herein, evaluating whether a patient is corticosteroid insensitive comprises obtaining a biological sample from the individual and conducting an in vitro assay. Studies have shown that cells from individuals can be corticosteroid insensitive, in vitro (e.g. in asthma at the cellular level, macrophages (Goleva et al (2006) Am J Respir Crit Care Med 173:607-616), epithelial cells (Hamilton et al., (2003) Clin. Exp. Allergy 33:233-240) and PBMCs (Mercado et al., (2011) Mol. Pharmacol. 80:1128-1135) obtained from patients have been reported to be corticosteroid insensitive in vitro. Studies have also shown that impaired glucocorticoid receptor nuclear localization is associated with increased phosphorylation, correlates with reduced corticosteroid sensitivity (Mercado et al. (2012) PLosOne 7(7):e4182).

In one embodiment, evaluating the corticoid sensitivity of disease in an individual comprises obtaining a biological sample from the individual and determining in vitro the presence of one or more inflammatory mediators and/or markers of ongoing inflammation or disease. In one embodiment, evaluating whether a patient is corticosteroid insensitive comprises obtaining a biological sample from the individual and determining the presence of one or more biomarkers associated with poor therapeutic response to corticosteroids, wherein the presence of one or more biomarkers associated with poor therapeutic response to corticosteroids indicates that the patient is corticosteroid insensitive. In one embodiment, the biomarker is the glucocorticoid receptor (GR). In one embodiment, GR nuclear localization or phosphorylation is assessed, wherein impaired nuclear localization (e.g. reduced GR translocation) and/or increased GR phosphorylation (e.g. at S226) or p38MAPKa/b activity is indicative of a poor therapeutic response. In one embodiment, evaluating whether a patient is corticosteroid insensitive comprises obtaining a biological sample from the individual and assessing whether cells (e.g. macrophages, epithelial cells, PBMC and/or other immune cells) are sensitive to a corticosteroid, wherein insensitivity indicates that the patient is corticosteroid insensitive.

In one embodiment, evaluating whether a patient is corticosteroid insensitive comprises evaluating symptoms of disease, e.g., through spirometry in respiratory disease, evaluating symptoms with and without corticosteroid treatment. In one embodiment, evaluating whether a patient is corticosteroid insensitive comprises treating the patient with (e.g. administering to the patient) a corticosteroid (e.g. a high dose corticosteroid regimen, a low-dose corticosteroid regimen, a short duration corticosteroid treatment), and observing an insufficient response, or dependence on the continued administration of corticosteroids. If disease is present (e.g. inadequately controlled) following such corticosteroid treatment, the patient can be corticosteroid-insensitive.

In one embodiment, a patient is treated with a corticosteroid and determined to be corticosteroid-insensitive if corticosteroid resistance observed during the first weeks (e.g. first 6 weeks, first 4 weeks) of treatment. In one embodiment, a patient is treated with a corticosteroid and determined to be corticosteroid-insensitive if disease is present (e.g. inadequately controlled) when high doses of a corticosteroid is administered. In one embodiment, a patient is treated with a corticosteroid and determined to be corticosteroid-insensitive if disease is present (e.g. inadequately controlled) when corticosteroid dose is tapered following a treatment at higher doses of corticosteroid (the patient has corticosteroid dependence).

In one aspect of any embodiment herein, the corticosteroid is selected from the group consisting of: hydrocortisone (Cortisol), cortisone acetate, prednisone, prednisolone, methylprednisolone, deflazacort, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide. In one aspect, the corticosteroid is dexamethasone. In one aspect, the corticosteroid is budenoside. In one aspect, the corticosteroid is a corticosteroid other than dexamethasone.

In one aspect, provided is a method of assessing or screening an anti-TLR3 agent, comprising providing an anti-TLR3 agent and evaluating whether the anti-TLR3 agent is capable of sensitizing a cell to the effects of a corticosteroid. The step of evaluation can be carried out for example according to any method which detects whether an anti-TLR3 agent renders an anti-inflammatory effect of a corticosteroid more efficient, e.g. an in vitro or in vivo assay, any assays disclosed herein.

In one aspect, the antibodies bind human TLR3 inhibit, TLR3 signalling, e.g. in a TLR3-expressing reporter cell, optionally in a dendritic cell (DC) (e.g. a myeloid DC (MdDC) or a monocyte derived DC). In one aspect, the disease to be treated involves cross-talk between immune cell populations, e.g. between DC and B- and/or T-cells.

In one aspect, the antibodies bind human TLR3 under acidic conditions, and in particular under conditions representative of that encountered in an acidified subcellular compartment of a cell (e.g. compartments of the endocytic pathway endosomal, lysosomal). Such acidic conditions are generally characterized by a pH lower than about pH 6.5, or between about pH 4.5 to 6.5, or about pH 5.6. In one aspect of any of the embodiments herein, the antibodies modulate, optionally inhibit, TLR3 signalling in an acidified subcellular compartment of a cell (e.g. compartments of the endocytic pathway endosomic, lysosomal).

In other aspects of any of the embodiments herein, the antibodies' bivalent binding affinity for TLR3 under acidic conditions can optionally be characterized by a mean K_{D} of no more than about (i.e. better affinity than) 100, 50, 10, 5, or 1 nanomolar, optionally subnanomolar or optionally no more than about 300, 200, 100 or 10 picomolar. In one embodiment, the antibodies have binding affinity (K_{D}) for a human TLR3 polypeptide at an acidic pH, i.e. a pH of about 5.6, of less than 10⁻⁹ M, optionally less than 10⁻¹⁰M. In another embodiment, the antibodies have binding affinity (K_{D}) for a human TLR3 polypeptide at a neutral pH, i.e. a pH of about 7.2, of less than 10⁻⁹ M, optionally less than 10⁻¹⁰M. In another embodiment, the antibodies have an affinity of less than 10⁻⁹ M, optionally less than 10⁻¹⁰M at both an acidic pH and at a neutral pH.

In one embodiment, the anti-TLR3 agent is an antibody of any of the following three classes of anti-TLR3 inhibitory antibodies with in vivo activity that have shown strong efficacy in inhibition of TLR3 in vivo:
(i) antibodies, exemplified by antibodies 11E1, 7G11, 31F6, 32C4, and 37B7, that bind human TLR3 in a region on the N-terminal of the TLR3 protein that is on the opposite terminal end of the principal dsRNA binding site (the C-terminal portion of the TLR3 polypeptide (optionally these antibodies can block binding of a dsRNA TLR3 ligand to the secondary (N-terminal) dsRNA binding site on the TLR3 polypeptide). Such antibodies include epitopes in the segment corresponding to residues 41-251 (e.g. 41-89, 41-120, 41-139). The individual residues bound by various such antibodies include 1, 2, 3 or more of residues 64, 65, 86, 89, 112, 113, 115, 117, 120, 137 or 139 of the TLR3 polypeptide of SEQ ID NO: 1;
(ii) antibodies, exemplified by mAb 15, that block binding of a dsRNA TLR3 ligand to the primary (C-terminal) dsRNA binding site on the TLR3 polypeptide. Such antibodies include epitopes in the segment corresponding to residues 416-619 of the TLR3 polypeptide of SEQ ID NO: 1. The individual residues bound by such antibodies may include 1, 2, 3 or more of residues K416, K418, L440, N441, E442, Y465, N466, K467, Y468, R488, R489, A491, K493, N515, N516, N517, H539, N541, S571, L595, and K619 of the TLR3 polypeptide of SEQ ID NO: 1; and
(iii) antibodies exemplified by antibodies 31C3 and 34A3, that inhibit TLR3 signalling without blocking the binding of a dsRNA TLR3 ligand to a TLR3 polypeptide, and/or by binding TLR3 at a site outside of the dsRNA TLR3 ligand binding site and causing conformational change to TLR3 such that TLR3 can no longer signal. Such antibodies include epitopes in the segment corresponding to residues 102 to 151 (e.g., 152 to 173, 174-191). The individual residues bound by various such antibodies include residue 182 of the TLR3 polypeptide of SEQ ID NO: 1, or residues 116 and/or 145 of the TLR3 polypeptide of SEQ ID NO: 1.

In one aspect of any of the embodiments herein, the anti-TLR3 antibody inhibits TLR3 signalling with or without blocking the binding of a TLR3 ligand to a TLR3 polypeptide, with or without blocking binding of a dsRNA TLR3 ligand to the principal (C-terminal) dsRNA binding site on the TLR3 polypeptide. The TLR3 ligand may be a naturally occurring or non-naturally occurring TLR3 ligand, optionally a dsRNA-based ligand such as polyAU (polyadenylic acid:polyuridylic acid) or polyIC (polyinosinic:polycytidylic acid). In particular, the antibodies are able to inhibit TLR3 signalling even when a TLR3 ligand such as dsRNA is already bound to the TLR3 polypeptide and/or when a TLR3-expression cell has been in contact with a TLR3 ligand. The antibodies are also able to inhibit TLR3 signalling even in a pre-activated condition, e.g., in the presence of IFNα. The antibodies will also have the advantage of binding TLR3 even if the C-terminal TLR3 ligand binding site is occupied by a dsRNA molecule thus potentially allowing broader overall binding. Antibodies that inhibit TLR3 signalling without blocking the binding of a dsRNA TLR3 ligand to a TLR3 polypeptide have been found to bind certain epitopes on TLR3. Consequently in one embodiment, an anti-TLR3 antibody may be characterized as having reduced binding to a TLR3 polypeptide having a mutation at residue 182 of the TLR3 polypeptide of SEQ ID NO: 1, in comparison to binding to a wild-type TLR3 polypeptide of SEQ ID NO: 1; optionally said mutation is a K182E mutation. In another aspect, the antibody binds to at least one residue in the segment corresponding to residues 152 to 173 of the TLR3 polypeptide of SEQ ID NO: 1. In another aspect, the antibody binds to at least one residue in the segment corresponding to residues 102 to 151 of the TLR3 polypeptide of SEQ ID NO: 1. In one aspect, the antibody specifically binds to at least one residue in the segment corresponding to residues 102-204 of the TLR3 polypeptide of SEQ ID NO: 1. Optionally, the antibody inhibits signalling by the TLR3 polypeptide. Optionally, the antibody binds to at least one residue in the segment corresponding to residues 174 to 191 of the TLR3 polypeptide of SEQ ID NO: 1. In one embodiment, the antibody binds residue 116, and/or residue 145 of the TLR3 polypeptide of SEQ ID NO: 1. In another embodiment, the antibody does not bind residue 116, and/or residue 145 of the TLR3 polypeptide of SEQ ID NO: 1. Optionally, the antibody does not bind residue 171, and/or residue 196 of the TLR3 polypeptide of SEQ ID NO: 1. Optionally, the antibody binds amino acid residue 182 of the TLR3 polypeptide of SEQ ID NO:1. Optionally, binding of the antibody to a TLR3 polypeptide having a mutation at residues 116, 141, 196 and/or residue 171 of the TLR3 polypeptide of SEQ ID NO: 1 is not substantially reduced, in comparison to binding to a wild-type TLR3 polypeptide of SEQ ID NO: 1; optionally said mutation is a K145E, D116R, N196A and/or E171A mutation. Optionally, binding of the antibody to a TLR3 polypeptide having a mutation at residue 182 of the TLR3 polypeptide of SEQ ID NO: 1 is reduced, in comparison to binding to a wild-type TLR3 polypeptide of SEQ ID NO: 1; optionally said mutation is a K182E mutation.

In one aspect of any of the embodiments herein, the anti-TLR3 antibody may have a heavy and/or light chain having one, two or three CDRs of an antibody selected from the group consisting of antibody 11E1, 7G11, 31F6, 32C4 or 37B7 (antibodies that block binding of a dsRNA TLR3 ligand to the secondary (N-terminal) dsRNA binding site on the TLR3 polypeptide). Antibodies that block binding of a dsRNA TLR3 ligand to the secondary (N-terminal) dsRNA binding site on the TLR3 polypeptide have been found to bind certain epitopes on TLR3. Consequently in one embodiment, an anti-TLR3 antibody may be characterized as having reduced binding to a TLR3 polypeptide having a mutation at residues 64 and/or residue 65 of SEQ ID NO: 1, and/or reduced binding to a TLR3 polypeptide having a mutation at residues 86 and/or residue 89 of SEQ ID NO: 1; has reduced binding to a TLR3 polypeptide having a mutation at residues 117 and/or residue 120 of SEQ ID NO: 1, and/or has reduced binding to a TLR3 polypeptide having a mutation at residues 137 and/or residue 139 of SEQ ID NO: 1, and/or has reduced binding to a TLR3 polypeptide having a mutation at residues 112, 113 and/or 115 of SEQ ID NO: 1 (in each case, in comparison to binding to a wild-type TLR3 polypeptide of SEQ ID NO: 1); and/or binds to at least one, two, three, four, five, six, seven or more residues in the segment corresponding to residues 41-251, 41-89, 41-120 or 41-139 of the TLR3 polypeptide of SEQ ID NO: 1.

In one aspect of any of the embodiments herein, the antibodies inhibit TLR3 signalling and block the binding of a TLR3 ligand to a TLR3 polypeptide, optionally by blocking binding of a dsRNA TLR3 ligand to the principal (C-terminal) dsRNA binding site on the TLR3 polypeptide. An example of such an antibody is mAb 15. In one aspect of any of the embodiments herein, the antibody may have a heavy and/or light chain having one, two or three CDRs of antibody mAb 15. Antibody mAb 15 (including variants thereof, e.g., mAb 15EVQ) is described, e.g. in WO2010/127113, the disclosure of which is incorporated herein by reference. Antibodies that block binding of a dsRNA TLR3 ligand to the primary (C-terminal) dsRNA binding site on the TLR3 polypeptide have been found to bind certain epitopes on TLR3. Optionally, the antibody binds 1, 2, 3, 4, 5 or more of residues K416, K418, L440, N441, E442, Y465, N466, K467, Y468, R488, R489, A491, K493, N515, N516, N517, H539, N541, S571, L595, and K619 of the TLR3 polypeptide of SEQ ID NO: 1, in comparison to binding to a wild-type TLR3 polypeptide of SEQ ID NO: 1. In one embodiment, an anti-TLR3 antibody may be characterized as having reduced binding to a TLR3 polypeptide having a mutation at K467, R488 and/or R489.

In one aspect of any of the embodiments herein, the anti-TLR3 antibody competes for binding to a TLR3 polypeptide with any one or any combination of monoclonal antibodies 11E1, 7G11, 31F6, 32C4, 37B7, mAb 15, 31C3 or 34A3, optionally under acid and/or neutral conditions. In one embodiment, an antibody competes for binding to a TLR3 polypeptide, optionally under acid and/or neutral conditions, with an antibody selected from the group consisting of:

(a) an antibody having respectively a VH and VL region of 11E1,

(b) an antibody having respectively a VH and VL region of 7G11,

(c) an antibody having respectively a VH and VL region of 31F6,

(d) an antibody having respectively a VH and VL region of 32C4,

(e) an antibody having respectively a VH and VL region of 37B7,

(f) an antibody having respectively a VH and VL region of mAb 15,

(g) an antibody having respectively a VH and VL region of 31C3, and

(h) an antibody having respectively a VH and VL region of 34A3.

In one aspect, the anti-TLR3 antibody has one or more (including any combination thereof, or all of) of the following properties:
a. has a subnanomolar affinity for a TLR3 polypeptide at an acidic pH, e.g. a pH less than about 6.5, or between about 4.5 to 6.5 or about pH 5.6;
b. inhibits TLR3 signalling in an inflammatory background, e.g. in the presence of inflammatory cytokines such as IFNα;
c. internalizes into a cell that expresses TLR3;
d. inhibits IP-10 secretion on DC (e.g. in human myeloid DC);
e. binds to a TLR3 polypeptide comprising an amino acid sequence of SEQ ID NO: 1; and/or
f. specifically binds a human TLR3 polypeptide expressed solely at the surface of a cell, wherein the antibody has an EC₅₀ of no more than 0.3 μg/ml, optionally no more than 0.2 μg/ml, optionally no more than 0.1 μg/ml, for binding to cells expressing TLR3 solely at the cell surface.

In one embodiment, the antibody is chimeric, e.g. contains a non-murine, optionally a human, constant region. In one embodiment, the antibody is human or humanized.

In one aspect of any of the embodiments, the antibody is an antibody fragment selected from Fab, Fab′, Fab′-SH, F(ab′)2, Fv, diabodies, single-chain antibody fragment, or a multispecific antibody comprising multiple different antibody fragments. In one aspect of any of the embodiments, the antibody does not comprise an Fc domain or is of an isotype that is not substantially bound by FcγR. In one embodiment, the antibody is of an IgG4 or IgG2 isotype. In one embodiment, the anti-TLR3 antibody comprises a heavy chain of human IgG4 isotype. In one embodiment, the anti-TLR3 antibody comprises an IgG4 heavy chain comprising a serine to proline mutation in residue 241, corresponding to position 228 according to the EU-index (Kabat et al., “Sequences of proteins of immunological interest”, 5^{th} ed., NIH, Bethesda, M L, 1991). Compositions comprising such antibodies can be characterized as having less than about 15%, such as less than about 10% (e.g., about 5% or less, about 4% or less, about 3% or less, or even about 1% or less) of IgG4 “half-antibodies” (comprising a single heavy chain/light chain pair). Such IgG4 “half-antibody” by-products form due to heterogeneity of inter-heavy chain disulphide bridges in the hinge region in a proportion of secreted human IgG4 (see Angal et al., Molecular Immunology, 30(1):105-108, 1993 for a description of IgG4 “half-antibodies”, S241P mutation, and related principles). This effect is typically only detectable under denaturing, non-reducing conditions.

Also provided are pharmaceutical formulations and kits comprising the anti-TLR3 agents and/or corticosteroids for uses according to the methods herein.

Diseases that can be treated using the methods and anti-TLR3 compositions include inflammatory or autoimmune disorders that are known to be potentially responsive to or treatable with corticosteroids, examples include: inflammatory rheumatisms (rheumatoid arthritis, juvenile rheumatoid arthritis, Still's disease, ankylosing spondylitis and other spondyloarthritides), inflammatory bowel diseases including Crohn's disease and ulcerative colitis, acute or chronic hepatitis for which corticosteroids are indicated, pulmonary conditions including asthma, chronic obstructive pulmonary disease, sarcoidosis and other interstitial lung diseases, such as idiopathic pulmonary fibrosis, non-specific interstitial pneumonitis and organizing pneumonia), primary or secondary glomerulonephritis for which corticosteroids are indicated (including but not limited to refractory nephrotic syndrome in adults); acute and chronic demyelinating polyneuropathies; myasthenia gravis; systemic lupus erythematosus (SLE); antiphospholipid syndrome, Sjögren's syndrome, systemic sclerosis, polymyositis, dermatomyositis; inflammatory muscle disorders, Sharp's syndrome; mixed connective tissue disease; chronic or sub-acute cutaneous lupus; psoriasis; epidermolysis bullosa and refractory mucosal lesions of pemphigus vulgaris; severe refractory atopic dermatitis; rosacea; primary vasculitis including but not limited to “ANCA Associated Vasculitis” (AAV, e.g., vasculitis associated with autoantibodies: the anti-neutrophil cytoplasmic antibodies (ANCA); these AAV include microscopic polyangitis, Churg-Strauss' syndrome and Wegener's granulomatosis), polyartertitis nodosa, Goodpasture's syndrome, giant cell arteritis and polymyalgia rheumatica, Behçet's disease, Schönlein-Henoch purpura, Takayasu's arteritis, Kawasaki disease; vasculitis secondary to any inflammatory disease; idiopathic thrombocytopenic purpura and autoimmune hemolytic anemia; thrombotic thrombocytopenic purpura and micrioangiopathic hemolytic anemia and hypereosinophilic syndrome.

The methods and anti-TLR3 compositions can also be used in the treatment or prevention of sepsis or acute or chronic Graft versus Host Disease. The ant-TLR3 agent and corticosteroid can be administered in combination to a subject who has received or who is expected to receive a transplant, e.g. a stem cell transplant, a hematopoietic stem cell transplant, an organ transplant.

These and additional advantageous aspects and features of the invention may be further described elsewhere herein.

### BRIEF DESCRIPTION OF THE DRAWINGS

FIG. 1 shows dose dependant inhibition of TLR3 signalling using a 293T-mouse TLR3 luciferase assay with the mouse anti-TLR3 antibody 28G7 (full line, black squares) compared to a control antibody with no activity (control, dotted line, opened squares).

FIGS. 2A-2B show in vivo dose effect of test anti-mouse antibodies on IL-6 secretion in sera of mice treated with poly(A:U) dsRNA. FIG. 2A shows the inhibition of IL-6 secretion (in pg/ml), 2 h post treatment with 20 μg of poly(A:U) dsRNA injected intravenously, for 100 μg dose of anti-mouse TLR3 antibody 28G7 (black triangles), in comparison with a control antibody (control-open lozenges) and a PBS treated group (black lozenges). FIG. 2B shows the inhibition of IL-6 secretion (in pg/ml), 2 h post treatment with 100 μg of poly(A:U), for 200 μg dose of anti-mouse TLR3 antibody 28G7 (black triangles) injected intravenously, in comparison with a control antibody (control-open lozenges) and a PBS treated group (black lozenges). Anti-mouse TLR3 antibodies were injected intra-peritoneally 3 h prior poly(A:U) treatment.

FIGS. 3A-3C show results of a rheumatoid arthritis mouse models. FIG. 3A shows the results of a preventive rheumatoid arthritis mouse model. FIG. 3B shows the results of a curative rheumatoid arthritis mouse model. FIG. 3C shows the results of a curative rheumatoid arthritis mouse model when mice are treated with PBS, a control antibody, 28G7 and an anti-TNFα antibody (Humira™).

FIGS. 4A-4B show results of the mouse colitis model. FIG. 4A shows the wall thickness measurements for the mice treated with saline (black dots), with TNBS only (black squares) with an anti-TNFα antibody and TNBS (black triangles), with 28G7 and TNBS (open dots), and with a control Ab and TNBS (open squares). FIG. 4B shows the macroscopic damage score for the mice treated with saline (black dots), with TNBS only (black squares) with an anti-TNFα antibody and TNBS (black triangles), with 28G7 and TNBS (open dots), and with a control Ab and TNBS (open squares). The anti-TLR3 antibody ameliorates the development of the disease (* p<0.05, ** p<0.01 vs saline).

FIG. 5 shows results of a CLP (cecal ligation and puncture—sepsis) mouse model. In this acute model, mice experience an acute infection, mimicking septic shock.

FIG. 6 shows reduction of IP-10 production in donor 2 in response to polyAU using anti-human TLR3 mAbs 31C3 or 34A3 in combination with methotrexate, dexamethasone or Humira®, showing that TLR3 mAbs reduce IP-10 compared with polyAU and polyAU in combination with methotrexate, dexamethasone or Humira®.

FIG. 7 shows reduction of IP-10 production in donor 1 in response to polyIC using anti-human TLR3 mAbs 31C3 or 34A3 in combination with methotrexate, dexamethasone or Humira®, showing that TLR3 mAbs reduce IP-10 compared with polyIC and polyIC in combination with methotrexate, dexamethasone or Humira®.

FIG. 8 shows reduction of IP-10 production in donor 2 in response to polyIC using anti-human TLR3 mAbs 31C3 or 34A3 in combination with methotrexate, dexamethasone or Humira®, showing that TLR3 mAbs reduce IP-10 compared with polyIC and polyIC in combination with methotrexate, dexamethasone or Humira®.

FIG. 9 shows the effects of 2 mg/mouse TNBS i.c. on body weight, when either untreated or treated with isotype control antibody, anti-TLR3 antibody, dexamethasone, or anti-TLR3 antibody+dexamethasone. Results are expressed as means+/−s.e.m. * p<0.05 compared to Control (saline) group.

FIG. 10 shows the effects of 2 mg/mouse TNBS i.c. on disease activity index, when either untreated or treated with isotype control antibody, anti-TLR3 antibody, dexamethasone, or anti-TLR3 antibody+dexamethasone. Results are expressed as means±s.e.m. * p<0.05 compared to Control (saline) group. ^{$}p<0.05 compared to TNBS Dexa/39B10 group. Combination treatment with both dexamethasone and the anti-TLR3 antibody significantly inhibited disease activity, while dexamethasone did not inhibit TNBS-induced disease activity.

FIGS. 11A and 11B show the effects of 2 mg/mouse TNBS enema on macroscopic damage score (11A) and wall thickness (11B), when either untreated or treated with isotype control antibody, anti-TLR3 antibody, dexamethasone, or anti-TLR3 antibody+dexamethasone. Results are expressed as means±s.e.m. * p<0.05 compared to Control group, $ indicates p<0.05 compared to Dexa/isotype control-TNBS group. the mice that had received the combo treatment dexamethasone+anti-TLR3 had a significantly lower colonic wall thickness compared to mice that had received dexamethasone+isotype control (FIG. 11B).

FIG. 12 shows the experimental setting used to demonstrate the ability of anti-TLR3 antibodies sensitize human epithelial bronchial cells or PBMC to corticosteroids.

FIG. 13 shows that anti-TLR3 antibodies sensitize human PBMC to corticosteroids, illustrated by reduction in IP-10 production in response to double stranded RNA.

FIGS. 14 and 15 show that anti-TLR3 antibodies sensitize human epithelial bronchial cells to corticosteroids, illustrated by reduction in IP-10 (FIG. 14) and IL-6 (FIG. 15) production in response to double stranded RNA in a virus-transformed lung epithelial BEAS-2B cell line model.

### DETAILED DESCRIPTION OF THE INVENTION

### Introduction

Described herein are methods, compositions, and kits which are suitable for restoring corticosteroid sensitivity, enhancing corticosteroid sensitivity and/or reversing the corticosteroid insensitivity in a subject experiencing corticosteroid dependence, corticosteroid resistance, or unresponsiveness or intolerance to corticosteroids. Corticosteroid insensitivity related conditions include, for instance, a range of immune-inflammatory disorders/diseases treated with corticosteroids when the therapy fails to achieve disease control or is not effective or intolerant or dependent or resistant to corticosteroids, and combinations thereof. The methods, compositions, and kits are effective to achieve the corticosteroid-sensitizer effects of steroid-sparing in corticosteroid-dependent patients, better responsiveness or tolerance to corticosteroids, achieving efficacy by using a lower dose of corticosteroid, preventing individuals at risk for developing corticosteroid refractory responses or resistance or exacerbations in response to antigen exposures, infections, exercise, or irritants, achieving optimal immune-functions, easier responses for the subject or patient when steroid administration is tapered or withdrawn, or in prolonged administration of corticosteroids, decreased risks for developing corticosteroid-related adverse events such as opportunistic infections and bone loss, and combinations thereof. The corticosteroid insensitivity related conditions include various conditions/disorders associated with and/or attributed to corticosteroid resistance, corticosteroid refractory responses, corticosteroid dependence and corticosteroid intolerance.

Provides are methods for treating an autoimmune or inflammatory disease in a subject in need thereof using an anti-TLR3 binding agent (e.g. an anti-TLR3 antibody) which inhibits TLR3 signalling, in combination with a corticosteroid. Also provided aremethods for treating relapses, attacks, or acute phases, occurring during the course of an inflammatory or autoimmune disease in a subject in need thereof using an anti-TLR3 antibody which inhibits TLR3 signalling. Also provided are methods for treating established inflammatory or autoimmune diseases in a subject in need thereof using an anti-TLR3 antibody which inhibits TLR3 signalling. Also provided are treatment regimens and treatment combinations that can be used for the treatment of inflammatory or autoimmune disease in a subject in need thereof using an anti-TLR3 antibody which inhibits TLR3 signalling (e.g. the anti-TLR3 antibody inhibits the TLR3 signalling induced by a TLR3 ligand). In one embodiment, the corticosteroid-sensitizing combination therapy is used to decrease the dosage of corticosteroid compared to a treatment regimen not comprising anti-TLR3 agent and corticosteroid is administered in a low-dose regimen, for example at a dose of less than 1 mg/kg per day. In one embodiment, the corticosteroid-sensitizing combination therapy is used to treat an otherwise corticosteroid-insensitive subject, and the corticosteroid is administered in a high-dose regimen (e.g., at a dose of at least 1 mg/kg per day, or a dose of less than 1 mg/kg per day).

### DEFINITIONS

As used herein, “TLR3 ligand” refers to any compound that can specifically bind to and alter the activity of TLR3 in vitro, ex vivo, or in vivo. The compound can be a naturally occurring ligand, e.g., generally dsRNA or viral dsRNA, or a synthetic ligand such as polyIC or polyAU. The compound can be any type of molecule, including inorganic or organic compounds or elements, including proteins (such as antibodies), nucleic acids, carbohydrates, lipids, or any other molecular entity. Further, such compounds can modulate TLR3 receptors in any way, including activating or inhibiting, and by any mechanism, including by binding to the receptor and triggering or shutting off activity in a manner similar to a naturally occurring ligand, or by binding to the receptor and blocking access to other ligands. Optionally, the ligand activates the receptor, and as such can be used to induce the production of cytokines by TLR3-expressing cells.

As used herein, the term “corticosteroid”, used interchangeably with “corticoid” or “glucocorticoid”, refers to a class of therapeutic agents that bind cytosolic glucocorticoid receptor (GR) and are useful in treatment of inflammatory conditions, including those resulting from infection, transplant rejection and autoimmune disorders. Corticosteroids suppress inflammation mainly as a result of both activation of anti-inflammatory genes and suppression of pro-inflammatory genes. The activation of anti-inflammatory gene expression starts as corticosteroid binds cytosolic GR, which is activated and translocates to the nucleus. Once in the nucleus, it binds to glucocorticoid response elements (GREs) and transcriptional coactivator molecules, and causes acetylation of core histones, which leads to the expression of anti-inflammatory genes. Inflammatory stimuli switch on multiple inflammatory genes that encode cytokines, chemokines, adhesion molecules, inflammatory enzymes, and receptors via pro-inflammatory transcription factors, such as nuclear factor κB (NFκB) and activator protein 1, and the recruitment of co-repressor molecules. Activated glucocorticoid receptors bind to the coactivators in the nucleus to inhibit histone acetyl transferase (HAT) activity directly and recruit histone deacetylase 2 (HDAC2), leading to suppression of the activated inflammatory genes. Corticosteroids include those that are naturally occurring, synthetic, or semi-synthetic in origin, and are typically characterized by the presence of a steroid nucleus of four fused rings, for example, as found in cholesterol, dihydroxycholesterol, stigmasterol, and lanosterol structures. Corticosteroid drugs include cortisone, cortisol, hydrocortisone (11(31 7-dihydroxy-21-(phosphonooxy)-pregn-4-ene3 20-dione disodium), dihydroxycortisone, dexamethasone (21-(acetyloxy)-9-fluoro-11P,17-dihydroxy-16a-methylpregna-1,4-diene-3,20-dione), and highly derivatized steroid drugs such as beconase (beclomethasone dipropionate, which is 9-chloro-11P,17,21, trihydroxy16-methylpregna-1,4 diene-3,20-dione 17,21-dipropionate). Other examples of corticosteroids include flunisolide, prednisone, prednisolone, methylprednisolone, triamcinolone, deflazacort and betamethasone.

The terms “corticosteroid resistant disease”, “corticosteroid resistance” and “corticosteroid resistant subject” as used herein are intended to refer to diseases and subjects that do not respond significantly to corticosteroid therapy prior to, or outside of, treatment accordance with the methods herein.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (VL) and variable heavy chain (VH) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the examples of classes of antibodies that can be employed, particularly IgG because they are the most common antibodies in the physiological situation and because they are most easily made in a laboratory setting. Optionally the antibody is a monoclonal antibody. Optionally an antibody is a humanized, chimeric, human, or otherwise-human-suitable antibody. “Antibodies” also includes any fragment or derivative of any of the herein described antibodies.

The term “specifically binds to” means that an antibody can bind in a competitive binding assay to the binding partner, e.g. TLR3, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody (e.g. 31C3 or 34A3), it means that the antibody competes with the monoclonal antibody in a binding assay using either recombinant TLR3 molecules or surface expressed TLR3 molecules. For example, if a test antibody reduces the binding of 31C3 or 34A3 to a TLR3 polypeptide or TLR3-expressing cell in a binding assay, the antibody is said to “compete” respectively with 31C3 or 34A3.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Ka is defined by 1/Kd. Examples of methods for determining the affinity of mAbs can be found in Harlow, et al., Antibodies: A Laboratory Manual, Cold Spring Harbor Laboratory Press, Cold Spring Harbor, N. Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of Biacore instruments.

A “determinant” designates a site of interaction or binding on a polypeptide.

The term “epitope” is defined as an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

A “humanized” or “human” antibody refers to an antibody in which the constant and variable framework region of one or more human immunoglobulins is fused with the binding region, e.g. the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The terms “Fc domain,” “Fc portion,” and “Fc region” refer to a C-terminal fragment of an antibody heavy chain, e.g., from about amino acid (aa) 230 to about aa 450 of human γ (gamma) heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5^{th} ed., United States Public Health Service, National Institute of Health, Bethesda, Md.).

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

An antibody that “binds” a common determinant designates an antibody that binds said determinant with specificity and/or affinity.

Treatment of Disease

Provided herein are methods for the treatment of an individual having an autoimmune or inflammatory disease, comprising administering to the individual (a) an anti-TLR3 agent that inhibits signalling by a TLR3 polypeptide (e.g. an anti-TLR3 antibody) and (b) a corticosteroid. In one embodiment, the individual has an autoimmune or inflammatory disease that has been declared for an extended period of time (e.g. more than one year), has signs of ongoing or active inflammation, has physical signs of disease (e.g. respiratory symptoms or impairment, joint swelling, lesions, neurological symptoms, etc.), has chronic disease, has severe disease (as assessed by applicable criteria, e.g. DAS or ACR criteria in rheumatoid arthritis) or has progressing disease. In one embodiment, the individual has a corticosteroid insensitive disease. Optionally the individual has disease that is not sufficiently controlled with high doses of corticosteroid treatment (e.g. more than 1 mg/kg per day, between 1 mg/kg and 10 mg/kg per day, e.g., by injection or oral administration, e.g., of prednisone equivalent). Optionally the individual has disease that is not sufficiently controlled with 1 mg/day, optionally 2 mg/day of corticosteroid treatment by inhalation.

In one embodiment, the disease is selected from the group consisting of rheumatoid arthritis, Juvenile idiopathic arthritis, multiple sclerosis, Crohn's disease or rectocolitis, Lupus erythematosus, hepatitis, chronic obstructive pulmonary disease (COPD) or asthma, ankylosing spondylitis and related diseases. In one embodiment, the disease is characterized by the presence of a TLR3 ligand (e.g. extracellular dsRNA). In one embodiment, the disease is characterized by the presence of detectable levels of a proteolytic enzyme, an inflammatory mediator, a marker of ongoing inflammation or a proinflammatory cytokine (e.g. TNF-α and/or interleukin-1 (IL-1)).

In another embodiment, the subject to be treated is suffering from a corticosteroid insensitive autoimmune or inflammatory disorder. Example of such disorders may include: refractory inflammatory bowel disease, such as refractory ulcerative colitis and children with severe Crohn disease, corticosteroid refractory asthma or glucocorticoid resistant asthma or symptomatic corticosteroid dependent asthma, chronic obstructive pulmonary disorder, desquamative interstitial pneumonia refractory to corticosteroid, refractory inflammatory myopathies, refractory myasthenia gravis, refractory pemphigus vulgaris, methotrexaterefractory rheumatoid arthritis (RA) patients, refractory nephrotic syndrome in adults, corticosteroid dependent systemic lupus erythematosus (SLE), primary Sjogren's syndrome, systemic vasculitis and polymyositis, chronic graft-versus-host disease, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory idiopathic thrombocytopenia purpura, refractory orbital myositis, refractory or recurrent lymphomas, critically ill patients with sepsis or acute respiratory distress syndrome (ARDS) or relative adrenal insufficiency, corticosteroid-dependent conditions (e.g., rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, etc.

The term “corticosteroid insensitivity” is intended to include, but is not limited to, corticosteroid resistance, corticosteroid dependence, corticosteroid refractory responses, corticosteroid intolerance, and other types of corticosteroid ineffectiveness. Corticosteroid resistance to the anti-inflammatory effects of corticosteroids is defined as inadequate or no improvement after treatment with corticosteroid. Corticosteroid dependence is defined as a condition that initially responds to corticosteroids but relapses quickly upon drug withdrawal or dose tapering. Corticosteroid refractory response is defined as a condition that does not respond to an adequate induction dose of corticosteroids. It includes relatively or totally refractory responses to corticosteroid therapy. Other types of corticosteroid ineffectiveness includes need for a high dose or very high dose treatment, “difficult to treat” and “do not respond well” or severe cases, and impaired in vitro and in vivo responsiveness. Corticosteroid intolerance is defined as toxicity of the therapy and/or risks for developing corticosteroid-related adverse events such as opportunistic infections and bone loss.

The anti-TLR3 agent can thus be used as a corticosteroid sensitizer. A corticosteroid sensitizer is a pharmaceutical agent and product that has a function in restoring corticosteroid sensitivity, enhancing corticosteroid sensitivity, reversing corticosteroid insensitivity, and protecting against loss of corticosteroid sensitivity, and used for treating, preventing, or ameliorating one or more of the symptoms of diseases or disorders associated with corticosteroid insensitivity (e.g., corticosteroid dependent or corticoid resistant or unresponsive or intolerant to corticosteroids). Therapeutic effects of the use of a corticosteroid sensitizer include any, but are not limited to, steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune functions, easier responses for the subject or patient when steroid administration is tapered or withdrawn, or after prolonged administration of corticosteroids, decreased risks for developing corticosteroid-related adverse events such as opportunistic infections, bone loss, pathologic fracture, diabetes, cataract, and combinations thereof.

The treatment methods herein can advantageously be used to treat established disease, e.g. a corticosteroid-insensitive established disease. “Established disease” refers to an autoimmune or inflammatory disease which has been declared for an extended period of time, e.g. more than one year. Depending on the specific disease, established disease also means a disease which is not controlled e.g. which is still progressing or for which the patient does not experience remission, in the presence or in the absence of a treatment. In one aspect, provided is a method for the treatment of an autoimmune or inflammatory disease in a patient, comprising: (a) determining whether said patient has an established disease; and (b) if said patient has an established disease, administering to said patient an effective dose of anti-TLR3 antibody in combination with a corticosteroid. Optionally, the corticosteroid is administered at a low dose. In one aspect, provided is a method for the treatment of an autoimmune or inflammatory disease in a patient, comprising: (a) determining whether said patient has an established disease that does not respond sufficiently to corticosteroid therapy, e.g. high dose corticosteroid therapy; and (b) if said patient has an established disease that does not respond sufficiently to corticosteroid therapy, administering to said patient an effective dose of anti-TLR3 antibody in combination with a corticosteroid.

Anti-TLR3 antibodies can also advantageously be used to treat chronic disease, e.g. a corticosteroid-insensitive chronic disease. Nonlimiting examples of chronic inflammatory disorder which can be treated include asthma, rubella arthritis, and chronic autoimmune diseases, such as systemic lupus erythematosus, psoriasis, inflammatory bowel disease, including Crohn's disease and ulcerative colitis, multiple sclerosis and rheumatoid arthritis. Chronic disease” refers to a disease that persists for an extended period of time. For instance, a chronic disease can be a disease lasting 3 months or more, as defined by the U.S. National Center for Health Statistics. In one aspect, provided is a method for the treatment of an autoimmune or inflammatory disease in a patient, comprising: (a) determining whether said patient has chronic disease; and (b) if said patient has chronic disease, administering to said patient an effective dose of anti-TLR3 antibody and a corticosteroid. Optionally, the corticosteroid is administered at a low dose. In one aspect, provided is a method for the treatment of an autoimmune or inflammatory disease in a patient, comprising: (a) determining whether said patient has chronic disease that does not respond sufficiently to corticosteroid therapy, e.g. high dose corticosteroid therapy; and (b) if said patient has chronic disease that does not respond sufficiently to corticosteroid therapy, e.g. high dose corticosteroid therapy, administering to said patient an effective dose of anti-TLR3 antibody in combination with a corticosteroid.

In another embodiment, the subject to be treated is suffering from an acute inflammatory disorder. Examples of acute inflammatory disorders including graft versus host disease, transplant rejection, septic shock, endotoxemia, Lyme arthritis, infectious meningitis (e.g., viral, bacterial, Lyme disease-associated), an acute episode of asthma and acute episodes of an autoimmune disease.

In another embodiment, the subject to be treated is suffering from an attack, crisis, exacerbation or flare, e.g. a corticosteroid-insensitive attack, crisis, exacerbation or flare, or a an attack, crisis, exacerbation or flare in a steroid-resistant disorder or patient. The terms “attack”, “crisis”, “exacerbation” and “flare”, designate a more rapid evolution of new symptoms or worsening of old symptoms related to an inflammatory or an autoimmune disease. Such phases last over a period of hours or days, as opposed to a slow progression of the disease that occurs over months and years. During such attacks, the patient experiences fever, pain, inflammatory syndrome (flu-like syndrome). In RA, the joints of the patient are swollen and painful. The patient can experience flu-like syndromes. A crisis can last from a few hours to many weeks. In Multiple Sclerosis, flare-ups can feature a new symptom or the worsening of an existing symptom but must last at least 24 hours to be considered a true exacerbation, a flare up denotes new lesions forming in the brain or spinal cord that disrupt neural transmission. Most flare-ups last a few days or weeks but can last for several months. Effects can for instance be: movement difficulties or spasms, balance and coordination problems; vision problems, uncoordinated eye movements, blurred vision or double vision, partial blindness during a flare-up; bladder and bowel symptoms; sexual problems, changes in mental function: memory loss, inattention and poor judgment or depression. In COPD, an exacerbation can be defined as “an event in the natural course of the disease characterized by a change in the patient's baseline dyspnea, cough, and/or sputum that is beyond normal day-to-day variations, is acute in onset and may warrant a change in medication in a patient with underlying COPD”. The patient experiencing an exacerbation has one of the following symptoms: increased cough and sputum production, change in the color and/or thickness of the sputum, wheezing, chest tightness, fever. In Crohn's disease or rectocolitis, a flare up is mainly the exacerbation of usual Crohn's disease symptoms: diarrhea, crampy abdominal pain, fever, loss of appetite. In one aspect, provided is a method for the treatment an autoimmune or inflammatory disease in a patient comprising: (a) determining whether said patient is experiencing an attack, crisis, exacerbation or flare; (b) if said patient experiences an attack, crisis, exacerbation or flare, administering to said patient an effective dose of an anti-TLR3 antibody and a corticosteroid. Optionally, the corticosteroid is administered at a low dose. In one aspect, provided is a method for the treatment an autoimmune or inflammatory disease in a patient comprising: (a) determining whether said patient is experiencing an attack, crisis, exacerbation or flare that does not respond sufficiently to corticosteroid therapy, e.g. high dose corticosteroid therapy; (b) if said patient experiences an attack, crisis, exacerbation or flare that does not respond sufficiently to corticosteroid therapy, e.g. high dose corticosteroid therapy, administering to said patient an effective dose of an anti-TLR3 antibody and a corticosteroid.

In another embodiment, the subject to be treated is suffering from a relapse. The term “relapse” refers to worsening of a patient's symptoms. A disease is relapsing when the health or condition of the patient worsens. It may be specified that the subject is suffering from a relapse while being treated with a corticosteroid. In one aspect, provided is a method for the treatment an autoimmune or inflammatory disease in a patient comprising: (a) determining whether said patient is experiencing a relapse; (b) if said patient experiences a relapse, administering to said patient an effective dose of an anti-TLR3 antibody and a corticosteroid. Optionally, the corticosteroid is administered at a low dose. In one aspect, provided is a method for the treatment an autoimmune or inflammatory disease in a patient comprising: (a) determining whether said patient is experiencing a relapse flare that does not respond sufficiently to corticosteroid therapy; (b) if said patient experiences a relapse flare that does not respond sufficiently to corticosteroid therapy, administering to said patient an effective dose of an anti-TLR3 antibody and a corticosteroid.

The exact dosage and regimen for administering anti-TLR3 agent and corticosteroid will necessarily depend upon the needs of the subject being treated, the type of treatment, the efficacy of the compound and the degree of disease severity in the subject. However, a nonlimiting example of a dosage range for an anti-TLR3 antibody is from about 0.01 to about 150 mg/kg body weight/day. Multiple dosages may be given so as to maintain TLR3 inhibition over an extended time period over which the corticosteroid is given, e.g. at least 2, 3, 4, 5 or 6 months, or more. The anti-TLR3 antibody is administered by subcutaneous, intramuscular or intravenous route.

In the methods herein, the anti-TLR3 agent is administered to a subject in combination with one or more corticosteroids. The term “in combination with” a corticosteroid is intended to include simultaneous administration of the agent and the corticosteroid, administration of the agent first, followed by the corticosteroid and administration of the corticosteroid first, followed by the anti-TLR3 agent. Corticosteroids that can be include, inter alia, short acting compounds, intermediate acting compounds and long acting compounds. Corticoids are typically classified by the duration of their tissue effects: short acting compounds (e.g., beclomethasone, flunisolide, hydrocortisone, cortisone), intermediate acting compounds (e.g., prednisone, prednisolone, methylprednisolone, triamcinolone, deflazacort) and long-acting compounds (e.g., dexamethasone, betamethasone). One or more corticosteroids can be administered to the subject by a route and at a dosage effective to achieve the desired therapeutic results.

For example, the pharmaceutical composition comprising a corticosteroid may be administered by subcutaneous, intravenous, intraperitoneal, intraarterial or intramuscular injection; rectally; by transdermally delivery; intravaginal delivery; or buccally; or by oral delivery. When an inhalation route of administration is used, delivery may optionally be, for example, via an aerosol spray or powder mixture in a pressurized pack or a nebulizer or in an inhaler.

The anti-TLR3 agent and the corticosteroid are administered to the subject in need of treatment according to standard routes of drug delivery well known in the art, the particular route and dosage of the anti-TLR3 agent and the corticosteroid being selected depending upon the needs of the subject being treated, the type of treatment, the efficacy of the compound and the degree of disease severity in the subject. The anti-TLR3 agent and the corticosteroid are administered at an “effective therapeutic dose”, which means that amount of the therapeutic composition which, when administered to a subject produces an amelioration of a disorder in comparison to those subjects which have not been administered the drug.

The exact dosage and regimen for administering a corticosteroid to the subject will necessarily depend upon the needs of the subject being treated, the type of treatment, the efficacy of the compound and the degree of disease severity in the subject. However, a nonlimiting example of a dosage range for corticosteroids is from about 0.05 mg/day to about 1000 mg/day, depending upon the particular corticosteroid used.

With respect to the frequency of administration of the corticosteroid, any frequency which achieves the desired result (e.g., steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune-functions, easier responses for the subject when steroid administration is tapered or withdrawn, or after prolonged administration of corticosteroids, decreased risks for developing corticosteroid-related adverse events such as opportunistic infections and bone loss, and combinations thereof) when used in combination with the anti-TLR3 agent may be used. The frequency of administration of the corticosteroid will optionally be determined, at least in part, by the particular corticosteroid and/or dosage form selected. In various embodiments, the corticosteroid is administered at least once daily or at least one per week, for example every other day, at least 3 times per week. Certain dosage regimens utilize alternate day administration (e.g., high dose intravenous pulse therapy).

Specific examples of corticosteroids include, but are not limited to, hydrocortisone (Cortisol), cortisone acetate, dexamethasone (hereinafter, “Dexamethasone”), prednisone, prednisolone, methylprednisolone, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide.

Corticosteroid formulations suitable for administration are well known in the art and commercially available. For example, dexamethasone acetate, 16 mg/ml aqueous suspension, is suitable for intramuscular injection in the treatment of rheumatoid, dermatological, ophthalmic, gastrointestinal, hematologic, neoplastic, allergic conditions and collagen disorders. Nonlimiting examples of dosages include 0.8 mg, 1.6 mg, 4 mg and 16 mg of dexamethasone per injection.

Hydroxycortisone is available as a sterile aqueous solution for intravenous, intramuscular, and subcutaneous injection and is a potent anti-inflammatory agent for conditions such as osteoarthritis, rheumatoid arthritis, juvenile rheumatoid arthritis, acute and chronic bursitis. Exemplary initial dosages can be from 15 mg to 250 mg per human subject per day. Exemplary dosages are oral or parenteral, and can be administered in half the daily dosage, administered twice per day, or other multiples. Hydrocortisone injection can be added to sodium chloride injection or dextrose injection and administered by intravenous drip. Hydrocortisone valerate, 0.2% by weight, is formulated as a cream for topical use under the name Westcort. Dosages can comprise application to affected areas several times daily as thin films.

Beconase (beclomethasone) is available for inflammation of the nasal passages and sinuses, for example, as 8.4 mg for 200 metered spray doses in a 0.042% aqueous suspension, delivered in metered doses of 100 mg containing 42 pg per metered dose. It can be delivered, for example, in an aqueous medium in suspension with microcrystalline cellulose, carboxymethylcellulose sodium, dextrose, benzalkonium chloride, polysorbate 80, and 0.25% v/w phenylethyl alcohol. Additional propellants and media are included in some formulations.

In certain embodiments in which an anti-TLR3 agent is coadministered with a corticosteroid, the agent is administered systemically to inhibit TLR3 systemically while the corticosteroid is administered either locally or systemically. For example, in certain embodiments when a phosphodiesterase IV inhibitor or a beta-2 agonist is additionally administered together with a corticosteroid, the phosphodiesterase IV inhibitor or beta-2 agonist is administered systemically, such as intravenously or orally, and the corticosteroid is administered either systemically or locally. In other examples, where a corticosteroid is administered by inhalation, a beta-2 agonist can be combined in an inhaler such that corticosteroid and beta-2 agonist are delivered simultaneously (e.g. Sybicort™, budenoside and formoterol inhalers).

Based on efficacy data collected during mice experiments using potent TLR3-blocking antibodies, a dose as low as 100 μg/mouse produces a therapeutic effect. Such dosage is equivalent to 4 mg/kg in the mouse and therefore 0.5 mg/kg in a human subject. Therefore the anti TLR3-antibody can be administered at a dosage comprised between 0.01 and 20 mg/kg, 0.05 and 20 mg/kg in human, optionally 0.1 and 10 mg/kg, further optionally y between 0.5 and 5 mg/kg (for example a unit dose of between about 25 mg and 500 mg).

An exemplary treatment regime entails administration of the anti-TLR3 antibody twice per week, once per week, once every two weeks, once every three weeks, once every four weeks, once a month, once every 2 to 3 months, or once every 3 to 6 months. Exemplary dosage regimens for an anti-TLR3 antibody include between 0.01 and 20 mg/kg, 0.05 and 20 mg/kg (optionally 0.1 and 10 mg/kg, further optionally between 0.5 and 5 mg/kg) body weight body weight via intravenous administration or subcutaneous injection.

The anti-TLR3 antibody is optionally administered at a dose that is suitable to induce substantially full TLR3 receptor saturation (90%, optionally 95% receptor saturation), e.g. saturation of TLR3 polypeptide expressed in targeted cells. As the TLR3 receptor is thought to dimerize before signalling, an inhibition of less than fully saturation by at least 20%, 30%, 40%, 50% receptor saturation may be useful in the treatment of a disease. In one embodiment, a dose of anti-TLR3 antibody resulting in at least about 20%, 30%, 40%, 50%, 90% or 95% receptor saturation is administered from about 2 times per week to about once per month, or from about once per month to about once per 2 months. The dose can be, e.g., administered at least 3 times, at least 6 times, or more. For example, the method may comprise administering an anti-TLR3 antibody at a dose and a dosing frequency achieving at least about 20%, 30%, 40%, 50%, 90% or 95% TLR3 receptor saturation on targeted cells for at least about two weeks, one month, 6 months, 9 months or 12 months. In one embodiment, a regimen results in sustained substantially full receptor saturation. A dose of anti-TLR3 antibody resulting in substantially full receptor saturation for a period of at least about 1 week, 2 weeks or 1 month is administered.

Receptor occupancy can be evaluated on human samples where target cells are present (e.g. whole blood, any tissue which is the site of an inflammation, synovial fluid). Saturation percentage of the TLR3 receptor can be measured by FACS analysis using methods known in the art, via intracellular staining since the TLR3 receptor is present in the cells. Alternatively, saturation percentage can be determined using a test of cytokine inhibition secretion profile in response to a TLR3 ligand such as a dsRNA (i.e. polyAU) in mononuclear cells (e.g., PBMCs) obtained from a patient. An efficient cytokine inhibition is correlated with an efficient therapeutic effect and the dosage can then be adapted for each patient. Cytokines that can be measured in this assay are for instance IP-10 or IL-6. In another embodiment, receptor saturation is assessed as receptor occupancy, for example by conducting free site and bound site assays. Briefly, free and bound TLR3 receptor levels are assessed on target cells from a biological sample obtained from an individual treated with the anti-TLR3 antibody, where a free site assay assesses unbound TLR3 by staining with PE-conjugated form of the anti-TLR3 antibody administered to an individual. A bound site assay assesses TLR3 polypeptides occupied by anti-TLR3 antibody by staining with a PE-conjugated mouse anti-human IgG4 monoclonal antibody (when the anti-TLR3 antibody is of human IgG4 isotype) that recognizes the anti-TLR3 antibody bound to the TLR3 polypeptides. In one embodiment, provided is a method for treating an individual comprising: (a) administering an anti-TLR3 antibody to an individual and (b) determining TLR3 receptor saturation in the individual, optionally further determining a dosage of anti-TLR3 antibody to be administered to the individual.

In some embodiments, the anti-TLR3 agent is administered prior to the administration of the corticosteroid. For example, when the anti-TLR3 agent is an antibody, the anti-TLR3 antibody can be administered approximately 0 to 30 days prior to the administration of the second therapeutic agent. In some embodiments, an anti-TLR3 antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, the anti-TLR3 agent is administered concurrently with the administration of the therapeutic agents. In some embodiments, the anti-TLR3 agent is administered after the administration of the second therapeutic agent. For example, an anti-TLR3 antibody can be administered approximately 0 to 30 days after the administration of the second therapeutic agent. In some embodiments, an anti-TLR3 antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

Rheumatoid Arthritis

Rheumatoid arthritis (RA) is a chronic and typically progressive inflammatory disease in which the synovial membrane is the primary site of inflammation. Bone destruction occurs with the progression of inflammation, resulting in deformation or damage of bones and cartilages. Rheumatoid arthritis sometimes develops into a wasting disease accompanying not only inflammation of synovial membranes or osteoarticular tissues, but also systemic inflammation, causing disorders in various organs and tissues, and may even lead to severe symptoms affecting life prognosis. Rheumatoid arthritis (RA) affects up to 1% of the adult population worldwide (Gabriel, Rheum Dis CHn North Am 27:269-81, 2001). Since rheumatoid arthritis often develops in people in their thirties and forties and gradually becomes advanced and aggravated during the middle to old age, it significantly affects daily life. The long-term prognosis of RA is poor, with as much as 50% of patients experiencing significant functional disability within 10 years from the time of diagnosis. (Keystone, Rheumatology, 44 (Suppl. 2): ii8-ii12 (2005)). Life expectancy is reduced by an average of 3-10 years. (Alamanos and Drosos). Patients with a high titer of rheumatoid factor (RF) (approximately 80% of patients) have more aggressive disease (Bukhari et al., Arthritis Rheum., 46: 906-912 (2002)), with a worse long-term outcome and increased mortality over those who are RF negative. (Heliovaara et al., Ann. Rheum. Dis., 54: 811-814 (1995)). Therefore, vigorous research and development of anti-rheumatic agents have been carried out.

The pathogenesis of chronic inflammatory bone diseases, such as RA, is not fully elucidated. TNF-α, IL-1β, and IL-1Ra gene polymorphisms are associated with increased RA susceptibility risk and disease severity. (Paradowska and Lacki, Centr Eur J Immunol., 31(3-4): 117-122 (2006)). IL-1 and TNF-α gene polymorphisms are associated with levels of anti-cytokine, including anti-TNF, clinical responses. Such diseases are accompanied by bone loss around affected joints due to increased osteoclastic resorption. This process is mediated largely by increased local production of pro-inflammatory cytokines. These cytokines can act directly on cells in the osteoclast lineage or indirectly by affecting the production of the essential osteoclast differentiation factor, receptor activator of NFκB ligand (RANKL), and/or its soluble decoy receptor, osteoprotegerin (OPG), by osteoblast/stromal cells. Tumor necrosis factor-alpha (TNF-α) is a major mediator of inflammation. Its importance in the pathogenesis of various forms of bone loss is supported by several lines of experimental and clinical evidence. However, TNF-α is not essential for osteoclastogenesis, erosive arthritis, or osteolysis, as these can occur in the absence of TNF-α.

In RA specifically, an immune response is thought to be initiated/perpetuated by one or several antigens presenting in the synovial compartment, producing an influx of acute inflammatory cells and lymphocytes into the joint. Successive waves of inflammation, also referred to as attacks, lead to the formation of an invasive and erosive tissue called pannus. This contains proliferating fibroblast-like synoviocytes and macrophages that produce proinflammatory cytokines such as TNF-α and interleukin-1 (IL-1). Local release of proteolytic enzymes, various inflammatory mediators, and osteoclast activation contributes to much of the tissue damage. There is loss of articular cartilage and the formation of bone erosions. Surrounding tendons and bursa may become affected by the inflammatory process. Ultimately, the integrity of the joint structure is compromised, producing disability.

Structural damage to joints is an important consequence of chronic synovial inflammation. Between 60% and 95% of patients with RA develop at least one radiographic erosion within 3-8 years of disease onset. (Paulus et al., J. Rheumatol., 23: 801-805 (1996); Hulsmans et al., Arthritis Rheum., 43: 1927-1940 (2000)). In early RA, the correlation between radiographic damage scores and functional capacity is weak, but after 8 years of disease, correlation coefficients can reach as high as 0.68. (Scott et al., Rheumatology, 39:122-132 (2000)).

Rheumatoid arthritis (RA) progresses in stages. The first stage is the swelling of the synovial lining, causing pain, warmth, stiffness, redness and swelling around the joint. Second is the rapid division and growth of cells, or pannus, which causes the synovium to thicken. In the third stage, the inflamed cells release enzymes that may digest bone and cartilage, often causing the involved joint to lose its shape and alignment, more pain, and loss of movement. A patient affected with the disease can experience a period of remission, without pain, and then a rheumatoid arthritis crisis, also named flare or attack, where the pain will increase. The methods herein propose to treat such patient experiencing a crisis to help them to deal with the pain.

The level of RA disease can be evaluated using different criteria. The most known criteria have been set up by the ACR (American College of Rheumatology). ACR criteria are indicated as ACR 20, ACR 50, and ACR 70. ACR criteria measure improvement in tender or swollen joint counts and improvement in three of the following five parameters: acute phase reactant (such as sedimentation rate), patient assessment, physician assessment, pain scale and disability/functional questionnaire.

The severity of the disease can also be measured by a score known as DAS (Disease Activity Score). DAS is a composite index of RA activity drawn up by EULAR (European League Against Rheumatism) initially developed for 44 joints for the numbers of joints with synovitis and the 53 Ritchie index sites. DAS is calculated according to the following formula:

DAS=[0.553938√Richie's index]+[0.06465√(number of joints with synovitis)]+[0.330 Ln(erythrocyte sedimentation rate)]+0.024

Ritchie's index covers 53 joints: temporomandibular, acromioclavicular, sternocostoclavicular, shoulder, elbow, wrist, metacarpophalangeal (MCP), proximal interphalangeal (PIP) in the fingers, hip, knee, ankle, subtalar, transverse tarsal, and metatarsophalangeal (MTP).

Three activity levels have been defined according to the value of DAS: RA with low activity level DAS 2.4, moderate active RA 2.4<DAS≦3.7, active RA>3.7. Remission threshold value defined for DAS is <1.6.

The primary objective of the methods of treatment herein is to control the activity of the disease and, also, to achieve remission, reduce pain, prevent and control joint destruction, prevent loss of function in everyday activities and at work, and optimise the patient's quality of life.

Current treatment options for RA include corticosteroids (prednisone; dexamethasone, methylprenisolone, Medrol®). They can be given orally, intravenously, intramuscularly or can be injected directly into the joint. Corticosteroids are useful in early disease as temporary adjunctive therapy while waiting for DMARDs to exert their anti-inflammatory effects. Corticosteroids are also useful as chronic adjunctive therapy in patients with severe disease that is not well controlled on NSAIDs and DMARDs. The usual dose of predinione is 5 to 10 mg daily. Although prednisone can be started at higher doses (15 to 20 mg daily), attempts should be made to taper the dose over a few weeks to less than 10 mg daily. Once started, corticosteroid therapy may be very difficult to discontinue and even at low doses. Some patients are very sensitive to the tapering of prednisone which is generally done slowly over a few weeks. Although a few patients can tolerate every other day dosing of corticosteroids which may reduce side effects, most require corticosteroids daily to avoid symptoms. Once a day dosing of prednisone is associated with fewer side effects than the equivalent dose given twice or three times daily. Generally steroids are given in the morning upon wakening to mimic the body's own steroid surge. Repetitive short courses of high-dose corticosteroids, intermittent intramuscular injections, adrenocorticotropic hormone injections, and the use of corticosteroids as the sole therapeutic agent are all to be avoided. Intra-articular corticosteroids (e.g., triamcinolone or methylprednisolone and others) are effective for controlling a local flare in a joint without changing the overall drug regime. Current recommendations for treatment of RA include early treatment with disease modifying anti-rheumatic drugs (DMARDs) after the diagnosis has been established. Non-steroidal anti-inflammatory drugs (NSAIDs), and until recently, COX-2 inhibitors have been widely used while waiting to confirm the diagnosis or later in the course of the disease in conjunction with DMARDs. Methotrexate is the most widely used DMARD, but other agents, including hydroxychloroquine, sulfasalazine, and leflunomide, are also prescribed.

Non-steroidal anti-inflammatory agents (NSAIDs). These drugs inhibit the generation of prostaglandins by blocking cyclooxygenase enzymes, COX-1 and COX-2. Prostaglandins are mediators of inflammation and pain but also have important roles in maintenance of normal body functions including protection from stomach acid, maintenance of kidney blood flow, and contributing to platelet stickiness and vascular function. COX-2 selective inhibitors selectively block prostaglandins generated via COX-2 which have prominent roles in inflammation. Many different NSAIDS are available, some over the counter including aspirin, ibuprofen (Advil®, Motrin®, Nuprin®) and naproxen (Alleve®) and many others are available by prescription including meloxicam (Mobic®), etodolac (Lodine®), nabumetone (Relafen®), sulindac (Clinoril®), tolementin (Tolectin®), choline magnesium salicylate (Trilasate®), diclofenac (Cataflam®, Voltaren®, Arthrotec®), Diflusinal (Dolobid®), indomethicin (Indocin®), Ketoprofen (Orudis®, Oruvail®), Oxaprozin (Daypro®), and piroxicam (Feldene®). Longer acting NSAIDs that allow daily or twice daily dosing may improve compliance. The NSAID class also includes drugs known as COX-2 inhibitors that are also effective in controlling inflammation. Only one of these agents is currently available in the United States (celecoxib, Celebrex®) while additional compounds are available in other countries (etoricoxib, Arcoxia®; lumiracoxib, Prexige®). These drugs were designed to decrease the gastrointestinal risk of NSAIDS, but concerns of possible increases in cardiovascular risk with these agents has led to the withdrawal of two of these drugs from the market (rofecoxib, Vioxx®; valdecoxib, Bextra®). While in some cases, lower doses of NSAIDS are effective, in rheumatoid arthritis and other forms of inflammatory arthritis a higher dose is often required to decrease inflammation. A lower dosage can initially be used if inflammation is mild, if mechanical pain is the major problem, if the patient is elderly or if the patient suffers from conditions that increase the risk for toxicity (see below). If a particular preparation is ineffective after a 4-week trial or is not tolerated, then another NSAID can be initiated. No one NSAID has been demonstrated to be better than another for the treatment of rheumatoid arthritis nor have the COX-2 agents been shown to be superior to traditional NSAIDS in terms of effectiveness.

Disease Modifying Anti-rheumatic Drugs (DMARDS): Although both NSAIDs and DMARD agents improve symptoms of active rheumatoid arthritis, only DMARD agents have been shown to alter the disease course and improve radiographic outcomes. DMARDs have an effect upon rheumatoid arthritis that is different and may be more delayed in onset than either NSAIDs or corticosteroids. In most cases, when the diagnosis of rheumatoid arthritis is confirmed, DMARD agents should be started. The presence of erosions or joint space narrowing on x-rays of the involved joints is a clear indication for DMARD therapy. The currently available drugs include: Methotrexate (Rheumatrex®, Trexall®), Hydroxychloroquine (Plaquenil®), Sulfasalazine (Azulfidine®), Leflunomide (Arava®), Tumor Necrosis Factor Inhibitors (e.g. anti-TNFalpha antibodies)—etanercept (Enbrel®, adalimumab (Humira®), and infliximab (Remicade®), T-cell Costimulatory Blocking Agents—abatacept (Orencia®), B cell Depleting Agents—rituximab (Rituxan®), Interleukin-1 (IL-1) Receptor Antagonist Therapy—anakinra (Kineret®), Intramuscular Gold, Other Immunomodulatory and Cytotoxic agents—azathioprine (Imuran®), cyclophosphamide, and cyclosporine A (Neoral®, Sandimmune®).

Methotrexate is now considered the first-line DMARD agent for most patients with RA. It has a relatively rapid onset of action at therapeutic doses (6-8 weeks), good efficacy, favorable toxicity profile, ease of administration, and relatively low cost. Methotrexate is effective in reducing the signs and symptoms of RA, as well as slowing or halting radiographic damage. Methotrexate is also effective in many other forms of inflammatory arthritis including psoriatic arthritis and other spondyloarthopathies, and is used in many other autoimmune diseases. Dosage: In a study comparing methotrexate to etanercept in early RA, methotrexate was started at a dose of 10 mg per week, and increased to 20 mg per week by week 8. This dosing regimen or regimens that start at even higher doses (up to 15 mg per week) with a dose escalation to 20 mg within the first three months is now fairly well accepted in clinical practice. Maximal dose is usually 25 mg per week but is sometimes increased further. Methotrexate can be given orally or by subcutaneous injection. The latter route of administration can be advantageous for patients who have methotrexate-associated nausea. Patients starting methotrexate should be carefully evaluated for renal insufficiency, acute or chronic liver disease, significant alcohol intake or alcohol abuse, leukopenia (low white blood cell counts), thrombocytopenia (low platelet counts), or untreated folate deficiency. Obesity, diabetes and history of hepatitis B or C are factors that have been suggested but not confirmed to increase methotrexate hepatotoxicity (liver injury). Salicylates (and other NSAIDs) and the antibiotic trimethoprim (Bactrim®, Septra®) block the renal excretion of methotrexate and increase serum levels with an increased risk of toxicity. If alternatives exist, concomitant use of methotrexate and trimethoprim is to be avoided. The coadministration of NSAIDS with methotrexate is routine in patients with rheumatoid arthritis and is considered safe by rheumatologists as long as liver function tests are closely monitored. Methotrexate can be combined safely with nearly every other FDA approved DMARDs for RA, including sulfasalazine, hydroxychloroquine, TNF inhibitors, abatacept, rituximab, anakinra, and leflunomide. In all clinical trials combining methotrexate with one of these DMARDs, no unexpected toxicities or synergistic toxicities were observed with the exception of higher liver toxicity with leflunomide which is also metabolized by the liver.

Treatment with an Anti-TLR3 Antibody and Corticosteroid

In one aspect, a patient having RA, and optionally having active inflammation and/or established or chronic RA, and/or experiencing a flare is treated with an anti-TLR3 antibody in combination with a corticosteroid. Optionally, established RA may be characterized as RA which has been progressing for over a year, or which has been progressing for less than a year but is unresponsive to a first disease modifying anti-rheumatic drug (DMARD). Established RA can also be assessed using the DAS or the CAS criteria. “RA and related diseases” refers to diseases that can cause or derive from the onset or evolution of rheumatoid arthritis such as e.g. episcleritis, pneumothorax, embolism and ischemic skin ulcer.

Corticosteroids (e.g., prednisone; dexamethasone, methylprenisolone, Medrol®) can be administered orally, intravenously, intramuscularly or can be injected directly into the joint while anti-TLR3 antibody can be injected or infused, e.g. via subcutaneous, intravenous, intramuscular, intra-articular, intra-synovial, intrasternal, intrathecal or intralesional routes. In one embodiment, a TLR3 antibody is administered intra-articularly, e.g., at the site of the inflammation. The corticosteroid may be administered at less than 10 mg/kg per day, optionally less than 1 mg/kg per day of prednisone equivalent, for example prednisone can be administered at 5 to 10 mg (total) daily. Dosing may be daily, or in some cases less frequently (e.g. alternate days). Optionally, the treatment using corticosteroid and anti-TLR3 agent can additionally comprise an additional therapeutic agent for combination use, for example a DMARD and/or NSAID. Optionally, the treatment regimen using corticosteroid and anti-TLR3 agent does not include combination treatment with a DMARD and/or NSAID.

Inflammatory Bowel Diseases (IBD)—Crohn's Disease—Ulceratice Colitis

Inflammatory Bowel Diseases are a series of diseases affecting the gastrointestinal tractus. Most common IBD are ulcerative colitis and Crohn's disease. They are also known as chronic inflammatory diseases of the intestine regional enteritis, rectocolitis and granulomatous ileocolitis. Ulcerative Colitis (UC) is an inflammatory disease limited to the colon and rectum. Crohn's disease is an inflammatory disease of the intestines that may affect any part of the gastrointestinal tract from mouth to anus (however usually sparing the rectum), causing a wide variety of symptoms. It primarily causes abdominal pain, diarrhea, vomiting, or weight loss. Both diseases (Crohn's disease and UC) may also cause complications outside of the gastrointestinal tract such as skin rashes, arthritis, inflammation of the eye, and tiredness, and augmentation of likelihood of a colon cancer.

Historically, Crohn's disease has been difficult to diagnose. In part, this is because its symptoms are similar to those of other bowel disorders, including ulcerative colitis and irritable bowel syndrome. A larger problem is that the small intestine has been difficult to examine using traditional methods.

Diagnosis tests include: non-invasive laboratory tests (anaemia and infection, liver function tests to screen for liver and bile duct problems, and stool studies to rule out bacterial, viral and parasitic infections), endoscopy, endoscopic ultrasound (EUS), capsule endoscopy, radiology such as Multiphase CT enterography, MR enterography (MRE).

Chronic inflammatory diseases of the intestine are rather hard to score. In rectocolitis, a coloscopy can provide a quite complete overview of the wounds in the colon, but in Crohn's disease, as wound can appear anywhere from oesophagus to rectum, patient evaluation is much more difficult to obtain. A scoring system has been set up to evaluate Crohn's disease: the Crohn's disease activity index (CDAI, see for review Sandborn W J et al. Gastroenterology 2002; 112:512). Score ranges from 0 to 600. Below 150 points, patients are scored as “very well”. Between 150 and 219, the disease is mildly active, between 220 and 449, the disease is moderately active. Above 450 points the disease is rated as very severe. However, such scoring may be patient dependant and more recently, another scoring system, the Crohn Disease Digestive Damage Score (CD₃S) or Lémann score has been established to score patients disease through more precise scientific values, such as a score established by tomodensitometry. (Pariente B. et al. Development of the Crohn's Disease Digestive Damage score, the Lernann score Innflamm Bowel Dis 2011; 127:1415-22.

Crohn's disease and ulcerative colitis evolve with crises, also known as flare-ups. Flare-ups can be mild or severe, brief or prolonged. Severe flare-ups can lead to intense pain, dehydration, and blood loss. Recurrent inflammation tends to appear in the same area of the intestine, but it may spread to adjacent areas after a diseased segment has been removed surgically. When Crohn's disease or ulcerative colitis causes a flare-up of gastrointestinal symptoms, the person may also experience inflammation of the joints (arthritis), inflammation of the whites of the eyes (episcleritis), mouth sores (aphthous stomatitis), inflamed skin nodules on the arms and legs (erythema nodosum), and blue-red skin sores containing pus (pyoderma gangrenosum). Even when Crohn's disease or ulcerative colitis is not causing a flare-up of gastrointestinal symptoms, the person still may experience pyoderma gangrenosum, while inflammation of the spine (ankylosing spondylitis), inflammation of the pelvic joints (sacroiliitis), inflammation inside the eye (uveitis), or inflammation of the bile ducts (primary sclerosing cholangitis) are liable to occur entirely without relation to the clinical activity of the bowel disease.

Current treatment options are restricted to controlling symptoms, maintaining remission, and preventing relapse. Treatment of Crohn's disease and ulcerative colitis involve first treating the acute symptoms of the disease, then maintaining remission. Treatment initially involves the use of medications to eliminate infections, generally antibiotics, and reduce inflammation, generally aminosalicylate anti-inflammatory drugs and corticosteroids. Surgery may be required for complications such as obstructions or abscesses, or if the disease does not respond to drugs within a reasonable time.

Aminosalicylate anti-inflammatory drugs include Mesalazine or mesalamine (Lialda™, Asacol™, Pentasa™, Salofalk™, Dipentum™ and Rowasa™), Sulfasalazine, which is converted to 5-ASA and sulfapyridine by intestinal bacteria. The sulfapyridine may have some therapeutic effect in rheumatoid arthritis. However, the sulfapyridine component is often the limiting factor in treatment of IBD because of high side-effect profile. 5-ASA compounds have been shown to be useful in the treatment of mild-to-moderate Crohn's disease. They are usually considered to be first line therapy for disease in the ileum and right side of the colon particularly due to their lower side effect profile compared to corticosteroids. They can also be administered intra-rectally.

Corticosteroid are used as current treatments in steroid enemas for treatment of rectal disease symptoms and more generally primarily for treatment of moderate to severe flares or attacks of Crohn's disease or ulcerative colitis. They are used more sparingly due to the availability of effective treatments with less side-effects. The most commonly prescribed oral corticosteroid is prednisone, which is typically dosed at 0.5 to 1 mg/kg for induction of remission (e.g. 40 mg/day or more). Intravenous steroids are used for cases refractory to oral steroids, or where oral steroids cannot be taken. Because corticosteroids reduce the ability to fight infection, care must be used to ensure that there is no active infection, particularly an intra-abdominal abscess before the initiation of steroids. Budesonide is an oral corticosteroid with limited absorption and high level of first-pass metabolism, meaning that less quantities of steroid enter into the bloodstream. It has been shown to be useful in the treatment of mild-to-moderate Crohn's disease and for maintenance of remission in IBD. Formulated as Entocort™, budesonide is released in the ileum and right colon, and is therefore has a topical effect against disease in that area. Budesonide is also useful when used in combination with antibiotics for active Crohn's disease. Finally, hydrocortisone or beclomethasone is also used orally or rectally to treat the distal manifestations of these enteric inflammations.

Corticosteroids are also used as maintenance therapy but are mostly insufficiently effective at a low dose.

Mercaptopurine immunosuppressing drugs can be used. Azathioprine is a first line steroid-sparing immunosuppressant. Azathioprine and 6-mercaptopurine (6-MP) are the most used immunosuppressants for maintenance therapy of IBD. They are purine anti-metabolites, meaning that they interfere with the synthesis of purines required for inflammatory cells. They have a duration of action of months, making it unwieldy to use them for induction of remission. Both drugs are dosed at 1.5 to 2.5 mg/kg, with literature supporting the use of higher doses. Azathioprine and 6-MP have been found to be useful for the following indications: maintenance therapy for people who are dependent on steroids, fistulizing disease, induction of remission in steroid refractory disease, maintenance of remission after surgery for Crohn's disease. Azathioprine is however a dangerous drug, with potential for inviting a host of potentially fatal infections, and is also listed by the FDA as a human carcinogen. Azathioprine can also induce bone marrow failure or pancreatitis. Azathioprine can be replaced by methotrexate which has however also potential toxicity, including bone marrow suppression and hepatotoxicitylnfliximab, marketed as Remicade™, a mouse-human chimeric antibody that targets tumour necrosis factor, can also be used. It is a monoclonal antibody that inhibits the pro-inflammatory cytokine tumour necrosis factor alpha. It is administered intravenously and dosed per weight starting at 5 mg/kg and increasing according to character of disease. Infliximab has found utility as follows: maintenance of remission for people with Crohn's disease and ulcerative colitis, induction of remission for people with Crohn's disease, and ulcerative colitis maintenance for fistulizing Crohn's disease. Adalimumab, marketed as Humira™, an antibody that targets tumour necrosis factor, can also be used. Adalimumab has been shown to reduce the signs and symptoms of, and is approved for treatment of, moderate to severe Crohn's disease (CD) in adults who have not responded well to conventional treatments and who have lost response to, or are unable to tolerate infliximab.

Cortico-resistant ulcerative colitis are often treated by intravenous cyclosporine, even if the above mentioned anti TNF antibodies are increasingly used as an alternative treatment; given their better short term safety profile and effectiveness as compared with cyclosporine.

Treatment with an Anti-TLR3 Antibody and Corticosteroid Combination

The anti-TLR3 agent and corticosteroid combination therapy can be used as maintenance therapy in established Crohn's disease or ulcerative colitis, as well as induction to reduce or abort a disease attack, thereby leading to an improvement of the patient's health and comfort. Thus, provided also is a method for treating a patient having a chronic inflammatory disease of the intestine comprising the step of assessing whether said patient is experiencing a flare-up or an attack, and if said patient is experiencing an attack, treating said patient with an effective amount of an anti-TLR3 agent and corticosteroid. The methods can also be used for the prophylactic treatment of a patient suffering from a Crohn's disease, thereby avoiding a flare up.

In one embodiment, a subject is treated with anti-TLR3 agent in combination with a corticosteroid, for example dosed at less than 5 mg/kg per day, at 1 mg/kg per day or less than 1 mg/kg per day, about 0.5 mg/kg per day or less than 0.5 mg/kg per day of prednisone-equivalent. For example an orally administered corticosteroid can be used, optionally prednisone, In one embodiment, a subject is treated with anti-TLR3 agent in combination with an injected or intravenously administered corticosteroid, for example dosed at less than 5 mg/kg per day, at 1 mg/kg per day or less than 1 mg/kg per day, about 0.5 mg/kg per day or less than 0.5 mg/kg per day of prednisone-equivalent. In one embodiment, the orally administered corticosteroid is formulated to be released in the ileum and right colon. In one embodiment, the corticosteroid is prednisone or budesonide. When the anti-TLR3 agent is an antibody, the antibody can be administered by injection or infusion.

Treatment with anti-TLR3 agent and corticosteroid can involve treatment with or without an additional treatment selected from the group consisting of an aminosalicylate anti-inflammatory drug, a mercaptopurine immunosuppressing drugs, an anti-TNFalpha agent (e.g. infliximab, adalimumab) and another immunomodulatory antibody.

Hepatitis and Autoimmune Hepatitis

Hepatitis is an inflammation of the liver characterized by the presence of inflammatory cells in the tissue of the organ. Hepatitis may occur with limited or no symptoms, but often leads to jaundice, anorexia and malaise. Hepatitis is acute when it lasts less than six months and chronic, or established when it persists longer. Hepatitis is established when it has been diagnosed for more than 6 months. A group of viruses known as the hepatitis viruses cause most cases of hepatitis worldwide, but it can also be due to toxins (notably alcohol, certain medications and plants), other infections, fatty liver accumulation and autoimmune diseases. Yin et al, Gastroenterology Research and Practice, Volume 2010 have reviewed the role of TLR3 in hepatitis.

Immunohistochemistry analyses showed that the expression of TLR3 was markedly increased in biliary epithelial cells at sites of ductular reaction in primary biliary cirrhosis and autoimmune hepatitis. A strong positive correlation between the mRNA levels of TLR3 and type I IFN in the liver was found in the patients with primary biliary cirrhosis, suggesting TLR3 signalling is involved in the pathogenesis of primary biliary cirrhosis. (M. Nakamura, K. Funami, A. Komori et al, Hepatology International, vol. 2, no. 2, pp. 222-230, 2008 and Y. Takii, M. Nakamura, M. Ito et al., Laboratory Investigation, vol. 85, no. 7, pp. 908-920, 2005).

Hepatitis can be of various origin, for instance, hepatitis can be diagnosed following an autoimmune dysregulation, hepatic dysfunction due to an overdose of alcohol, or be a side effect of a immunosuppressive treatment.

Autoimmune hepatitis is an inflammation of the liver without a specific cause. The condition is chronic and progressive. Although the disease is chronic, many patients with autoimmune hepatitis present acutely ill with jaundice, fever and sometimes symptoms of severe hepatic dysfunction, a picture that resembles acute hepatitis. Patients usually present with evidence of moderate to severe hepatitis with elevated serum ALT and AST activities in the setting of normal to marginally elevated alkaline phosphatase and gamma-glutamyltranspeptidase activities. Blood tests identify ANA or smooth muscle antibodies (SMA) in the majority of patients (60%). More than 80% of affected individuals have increased gamma globulin in the blood. Autoimmune hepatitis usually occurs in women (70%) between the ages of 15 and 40 and an estimated 70% will be forced to continue drug therapy for the rest of their lives or in some cases be candidates for a liver transplant. An estimated 10% to 30% will be able to discontinue all drug therapy after a remission of four years.

Serum protein electrophoresis and testing for autoantibodies are of central importance in the diagnosis of autoimmune hepatitis. Patients with one subtype of autoimmune hepatitis have serum gamma-globulin concentrations more than twice normal and sometimes antinuclear antibodies and/or anti-smooth muscle (anti-actin) antibodies. Patients with another subtype may have normal or only slightly elevated serum gamma-globulin concentrations but will have antibodies against a particular cytochrome p450 isoenzyme that are called anti-LKM (liver kidney microsome).

The most common symptoms of autoimmune hepatitis are fatigue, abdominal discomfort, aching joints, itching, jaundice, enlarged liver, and spider angiomas (tumors) on the skin. Patients may also have complications of more advanced chronic hepatitis with cirrhosis, such as ascites (abdominal fluid) or mental confusion called encephalopathy. A liver biopsy is important to confirm the diagnosis and provide a prognosis. Liver biopsy may show mild chronic active hepatitis, more advanced chronic active hepatitis with scarring (fibrosis), or a fully developed cirrhosis.

Current treatment options include corticosteroids. Patients in whom a diagnosis of autoimmune hepatitis is suspected should have a liver biopsy. If the biopsy is consistent, treatment with steroids (prednisone or prednisolone) and azathioprine (Imuran) is begun immediately. These are tapered over the next 6 to 24 months depending upon the patient's course. This medical therapy has been shown to decrease symptoms, improve liver tests, and prolong survival in the majority of patients. Therapy is usually begun with prednisone 30 to 40 mg per day and then this dosage is reduced after a response is achieved. The standard dosage used in the majority of patients is prednisone 10-15 mg per day, either alone or with azathioprine 50 mg per day. Higher doses of prednisone given long-term are associated with an increase in serious side effects, including: hypertension, diabetes, peptic ulcer, bone thinning, and cataracts. Lower doses of prednisone may be used when combined with azathioprine. If immediate liver biopsy is contraindicated because of a prolonged prothrombin time or thrombocytopenia, steroids and azathioprine should be started prior to biopsy if the diagnosis of autoimmune hepatitis is likely based on clinical criteria (e.g. a young woman with severe hepatitis, elevated serum gamma-globulin concentration, negative risk factors and serologies for viral hepatitis). The patient will often rapidly improve and biopsy should be performed to confirm the diagnosis as soon as the prothrombin time decreases and platelet count increases to within safe ranges. Azathioprine at a relatively high dose (2 mg per kilogram of body weight) induces a risk of development of a cancer.

The goal of treatment of autoimmune hepatitis is to cure or control the disease. About two thirds to three quarters of patients with autoimmune hepatitis respond to treatment based on the return of serum ALT and AST activities to normal and an improved biopsy after several months. Long-term follow-up studies show that autoimmune hepatitis appears more often to be a controllable rather than a curable disease, because the majority of patients relapse within six months after therapy is ended. Therefore, most patients need long-term maintenance therapy. Some patients relapse as corticosteroids and azathioprine doses are tapered or stopped and need chronic maintenance medications. Not all patients with autoimmune hepatitis respond to prednisone treatment. Approximately 15-20% of patients with severe disease continue to deteriorate despite initiation of appropriate therapy. Such patients are unlikely to respond to further medical therapy under standard practice, and liver transplantation should be considered. Over the long term, many patients develop cirrhosis despite having a response to treatment, and patients who do not respond to treatment will almost always progress to cirrhosis. If end-stage liver disease develops, orthotopic liver transplantation is an effective procedure.

Treatment with an Anti-TLR3 Antibody and Corticosteroid Combination

In one embodiment, a subject is treated with anti-TLR3 agent in combination with an orally administered corticosteroid, e.g. prednisone, for example dosed at less than 1 mg/kg per day, at 40 mg per day or less, or at 15 or 10 mg per day or less. In one embodiment, a subject is treated with anti-TLR3 agent in combination with an injected or intravenously administered corticosteroid, for example dosed at less than 1 mg/kg per day. In one embodiment, the combination treatment is administered to a patient that whose disease relapsed or did not respond well to previous treatment with a corticosteroid (e.g. a corticosteroid such as prednisone alone or together with azathioprine). When the anti-TLR3 agent is an antibody, the antibody can be injected or infused via subcutaneous, intravenous or intramuscular routes.

COPD

Chronic obstructive pulmonary disease (COPD), also known as chronic obstructive lung disease (COLD), chronic obstructive airway disease (COAD), chronic airflow limitation (CAL) and chronic obstructive respiratory disease (CORD), refers to chronic bronchitis and emphysema, a pair of commonly co-existing diseases of the lungs in which the airways become narrowed. In clinical practice, COPD is defined by its characteristically low airflow on lung function tests; This airflow obstruction is only partly reversible and usually progressive and associated with an a inflammatory response of the lung to noxious particles or gas, most commonly from tobacco smoking. The natural course of COPD is also frequently characterized by occasional sudden worsening of symptoms called acute exacerbations, most of which are caused by infections or air pollution.

A chronic obstructive pulmonary disease (COPD) diagnosis is confirmed by spirometry. Diagnosis of COPD should be considered in any patient who has symptoms of a chronic cough, sputum production, dyspnea and a history of exposure to risk factors for the disease. Because COPD develops slowly, it is most frequently diagnosed in people aged 40 years or over. COPD leads to a gradual decline of the forced expiratory volume, which often leads to a chronic respiratory failure. It is also frequently associated with systemic manifestations, severe comorbidities including not only lung cancer but also depression, skeletal muscle wasting, cachexia, osteoporosis, diabetes and ischemic heart disease, COPD is currently the fourth leading cause of death.

Current treatments for COPD include: Short-acting bronchodilators, both beta agonists and anticholinergics, are the mainstay of therapy for COPD, long-acting bronchodilators are indicated for moderate to severe COPD. Currently two beta agonists are available. Another treatment option is inhaled corticosteroids (e.g. dexamethasone, for example dexamethasone nasal spray such as Dexacort™) which are recommended, as combination with long-acting bronchodilators for patients with moderate to severe COPD with frequent exacerbations (incidents which worsen symptoms). Systemic corticosteroids (IV or pills) are beneficial for treatment of severe exacerbations. Antibiotics may be beneficial for treatment of exacerbations. Theophylline in low doses may reduce frequency of exacerbations in patients who tolerate it, despite of the strong reported side effects. An oral phosphodiesterase-4 inhibitor, roflumilast, was recently approved as maintenance treatment of severe COPD, associated with chronic bronchitis in adult patients with a history of frequent exacerbations as add on to bronchodilator treatment. Other compounds currently in development are also suitable for a combination in the treatment methods, including NVA23 and QVA149, a combination of indacaterol and NVA237, an inhaled muscarinic receptor antagonist (Novartis AG), indacaterol (QAB149), an adrenergic receptor beta 2 agonist (Novartis), and Relovair fluticasone furoate/vilanterol (GW685698/GW64244) (GlaxoSmith-Kline plc), a fixed dose combination of an inhaled corticosteroid and a long-acting adrenergic receptor beta 2 agonist (LABA).

Treatment with an Anti-TLR3 Antibody and Corticoid

A patient having COPD can be evaluated to assess the presence, stage, evolution or rating of disease. An individual suspected of having COPD, disease can be evaluated using spirometry, postbronchodilator spirometry, lung volumes, and diffusion capacity. Optionally, blood gases, optionally arterial blood gases are measured. A determination that a patient has COPD (or an exacerbation), indicates that the patient can be treated with an anti-TLR3 antibody and corticoid combination therapy.

In one embodiment, the subject with an exacerbation of his COPD is treated with an anti-TLR3 agent in combination with an orally administered corticosteroid, e.g. prednisone, for example dosed at between 0.5 mg/kg per day and 1 mg/kg per day, or optionally 1 mg/kg or more per day. In one embodiment, the subject is treated with an anti-TLR3 agent in combination with an inhaled administered corticosteroid, e.g. beclomethasone, for example dosed at less than 1000 μg per day. In one embodiment, the subject with an exacerbation of COPD is treated with an anti-TLR3 agent in combination with an injected or infused corticosteroid, e.g. prednisone, for example dosed at less than 5 mg/kg per day, or less than 1 mg/kg per day. When the anti-TLR3 agent is an antibody, the antibody can be injected or infused via subcutaneous, intravenous or intramuscular routes.

Asthma

Asthma is a common chronic disorder of the airways that is complex and characterized by variable and recurring symptoms, airflow obstruction, bronchial hyperresponsiveness, and an underlying inflammation. The interaction of these features of asthma determines the clinical manifestations and severity of asthma and the response to treatment. Asthma is a generally understood as a chronic inflammatory disorder of the airways in which many cells and cellular elements play a role: in particular, mast cells, eosinophils, T lymphocytes, macrophages, neutrophils, epithelial cells, and smooth muscle cells. In susceptible individuals, this inflammation causes recurrent episodes of wheezing, breathlessness, chest tightness, and coughing, particularly at night or in the early morning. These episodes are usually associated with widespread but variable airflow obstruction that is often reversible either spontaneously or with treatment. The inflammation also causes an associated increase in the existing bronchial hyperresponsiveness to a variety of stimuli. Reversibility of airflow limitation may be incomplete in some patients with asthma.

Asthma is generally diagnosed by presence of episodic symptoms of airflow obstruction or airway hyperresponsiveness. Treatment with anti-inflammatory drugs can, to a large extent, reverse some of these processes, and airflow obstruction is at least partially reversible, in the absence of alternate diagnoses. A careful medical history, physical examination, pulmonary function tests (e.g. spirometry), and additional tests are carried out to ensure a correct diagnosis of asthma.

Successful response to therapy often requires weeks to achieve and, in some situations, may be incomplete. For some patients, the development of chronic inflammation may be associated with permanent alterations in the airway structure—referred to as airway remodeling—that are not prevented by or fully responsive to currently available treatments. Therefore, the paradigm of asthma has been expanded from bronchospasm and airway inflammation to include airway remodeling. Pharmacologic therapy is used to prevent and control asthma symptoms, improve quality of life, reduce the frequency and severity of asthma exacerbations, and reverse airflow obstruction. Asthma medications are categorized into two general classes: long-term control medications taken daily on a long-term basis to achieve and maintain control of persistent asthma (these medications are also known as long-term preventive, controller, or maintenance medications) and quick-relief medications taken to provide prompt reversal of acute airflow obstruction and relief of accompanying bronchoconstriction (these medications are also known as reliever or rescue medications). Patients who have persistent asthma require both classes of medication. Among long-term medications are Corticosteroids, Cromolyn sodium and nedocromil, immunomodulators (e.g. anti-IgE antibodies), leukotriene modifiers, long-acting beta-adrenoceptor agonists (or long-acting β₂ agonists) (LABAs) such as salmeterol and formoterol; LABAs are often used in combination with corticosteroids.

Corticosteroids are generally administered by inhalation for maintenance therapy, and by oral or intravenous administration in case of exacerbations. Chronic administration of oral systemic can be used as a long-term-control medication in cases of severe asthma. While studies have shown that, for most patients whose asthma has been well controlled for at least 2 months by a high dose of a corticosteroid alone, a 50 percent reduction in dose does not lead to loss of control, guidelines indicate that treatment with corticosteroid cannot necessarily be stopped altogether as asthma control in most patients can worsen within a few weeks when treatment is discontinued. Such patients who need continued treatment may benefit from combination therapy with corticosteroids and anti-TLR3 agent. Corticosteroid treatment may be increased temporarily in response to some index of worsening asthma, although the effectiveness of this adjustable dose approach may be a function of timing or of dose, and exacerbations are not known to be consistently responsive to increased corticosteroid dosage.

Decreased corticosteroid dosage regiments have been developed using an inhaler containing both budesonide as corticosteroid and formoterol (a LABA with a rapid onset of action). It has been reported that use of a low dose of budesonide from this combination inhaler twice daily (maintenance therapy) plus additional use for relief of symptoms was associated with a lower rate of asthma exacerbations and a lower cumulative dose of budesonide. Such combinations can be used further with an anti-TLR3 agent.

Treatment with an Anti-TLR3 Antibody and Corticoid

In one embodiment, the subject is treated with an anti-TLR3 agent in combination with a corticosteroid, wherein the dose of the corticosteroid is less than 1 mg/kg per day of prednisone equivalent.

In one embodiment, the subject having asthma is treated with an anti-TLR3 agent in combination with an inhaled corticosteroid, e.g. beclomethasone dipropionate, budesonide, flunisolide, fluticasone propionate, mometasone furoate, triamcinolone acetonide, or a similar or equivalent corticosteroid. Optionally, the corticosteroid is administered in combination (further) with a long-acting β2-agonist, e.g. formoterol.

In one embodiment, the subject is treated with an anti-TLR3 agent in combination with an inhaled corticosteroid, wherein the dose of the corticosteroid is less than 2 mg per day, optionally less than 1 mg per day, optionally of beclomethasone equivalent. In one embodiment, the dose of inhaled corticosteroid is between 0.1 mg per day and 2 mg/day, optionally of beclomethasone equivalent. Equivalent doses between corticosteroids and formulations will vary and it will be appreciated that the skilled practitioner will determine the suitable dose for the particular inhaled corticosteroid. For example, in combination with an anti-TLR3 agent, beclomethasone hydrofluoroalkane (HFA) can be administered at 80-240 μg/day, corresponding to a dosage of Budesonide dry powder inhaler (DPI) of 180-600 μg/day, Flunisolide at 500-1000 μg/day, Flunisolide HFA at 320 μg/day, Fluticasone HFA/metered dose inhaler (MDI) at 88-264 μg/day or DPI at 100-300 μg/day, Mometasone DPI at 200 μg/day and Triamcinolone acetonide at 300-750 μg/day. For example, in combination with an anti-TLR3 agent, beclomethasone hydrofluoroalkane (HFA) can be administered at 240-480 μg/day, corresponding to a dosage of Budesonide dry powder inhaler (DPI) of 600-1200 μg/day, Flunisolide at 1000-2000 μg/day, Flunisolide HFA at 320-640 μg/day, Fluticasone HFA/metered dose ihaler (MDI) at 264-440 μg/day or DPI at 300-500 μg/day, Mometasone DPI at 400 μg/day and Triamcinolone acetonide at 750-1500 μg/day. Further, in combination with an anti-TLR3 agent, inhaled corticosteroids can be administered at non-low dose regimens in patients that would not otherwise have adequate disease control. For example beclomethasone hydrofluoroalkane (HFA) can be administered in a dosage of at least 480 μg/day, corresponding to a dosage of Budesonide dry powder inhaler (DPI) of at least 1200 μg/day, Flunisolide of at least 2000 μg/day, Flunisolide HFA of at least 640 μg/day, Fluticasone HFA/metered dose ihaler (MDI) of at least 440 μg/day or DPI of at least 500 μg/day, Mometasone DPI of at least 400 μg/day and Triamcinolone acetonide of at least 1500 μg/day.

In one embodiment, the subject having asthma is treated with an anti-TLR3 agent in combination with a corticosteroid administered systemically, e.g. orally, for example Methylprednisolone, Prednisolone or prednisone, or a similar or equivalent corticosteroid. In an optional embodiment, the corticosteroid is administered at less than 5 mg/kg per day, or less than 1 mg/kg per day.

In one embodiment, the subject is treated with an anti-TLR3 agent in combination with an injected or infused corticosteroid, e.g. prednisone, for example dosed at less than 1 mg/kg per day.

When the anti-TLR3 agent is an antibody, the antibody is typically injected or infused via subcutaneous, intravenous, or intramuscular, routes.

Lupus Erythematosus

Four main types of lupus exist—systemic lupus erythematosus, discoid lupus erythematosus, drug-induced lupus erythematosus, and neonatal lupus erythematosus. Of these, systemic lupus erythematosus is the most common and serious form of lupus.

Discoid lupus erythematosus (DLE) is a chronic skin condition of sores with inflammation and scarring favoring the face, ears, and scalp and at times on other body areas. These lesions develop as a red, inflamed patch with a scaling and crusty appearance. The center areas may appear lighter in color with a rim darker than the normal skin.

Drug-induced lupus erythematosus (DIL or DILE) is an autoimmune disorder caused by chronic use of certain drugs. These drugs cause an autoimmune response producing symptoms similar to those of SLE. There are 38 known medications to cause DIL but there are three that report the highest number of cases: hydralazine, procainamide, and isoniazid. While the criteria for diagnosing DIL has not been thoroughly established, symptoms of DIL typically present as myalgia and arthralgia. Generally, the symptoms recede after discontinuing use of the drugs.

Neonatal lupus erythematosus presents in infants, most often girls, born to mothers who carry the Ro/SSA antibody. The infants have no skin lesions at birth, but develop them during the first weeks of life.

Systemic lupus erythematosus is a chronic systemic autoimmune disease that can affect any part of the body. As occurs in other autoimmune diseases, the immune system attacks the body's cells and tissue, resulting in inflammation and tissue damage. SLE most often harms the joints, skin, kidneys, nervous system, blood cells, heart, lungs, blood vessels. The course of the disease is unpredictable, with periods of illness (flares) alternating with remissions. The disease occurs nine times more often in women than in men, especially in women in child-bearing years ages 15 to 35, and is more common in those also of non-European descent. SLE is treatable through addressing its symptoms, mainly with cyclophosphamide, corticosteroids and immunosuppressants. Severe lupus, susceptible to induce end organ damage, or even to be life-threatening are not rare, representing approximately 40% of all SLE cases. Most frequent severe damage consist of glomerulonephritis, high serum creatinine, hypertension, nephrotic syndrome, anemia and hypoalbuminemia are poor prognostic factors. The ANA is the most sensitive screening test for evaluation, whereas anti-Sm (anti-Smith) is the most specific. dsDNA titer is sometimes useful to monitor disease flares or response to treatment. Some physicians make a diagnosis on the basis of the American College of Rheumatology (ACR) classification criteria. The criteria, however, were established mainly for use in scientific research including use in randomized controlled trials which require higher confidence levels, so some people with SLE may not pass the full criteria. The American College of Rheumatology established eleven criteria in 1982, revised in 1997 as a classificatory instrument to operationalize the definition of SLE in clinical trials.

Current treatment of SLE involves preventing flares and reducing their severity and duration when they occur. Treatment can include corticosteroids and anti-malarial drugs. Frequent types of lupus nephritis such as proliferative glomerulonephritis require bouts of cytotoxic drugs. These drugs include cyclophosphamide and mycophenolate which are administered as combination with high doses of corticosteroids. Disease-modifying anti-rheumatic drugs (DMARDs) are used preventively to reduce the incidence of flares, the process of the disease, and lower the need for steroid use; when flares occur, they are treated with corticosteroids. Corticosteroids are typically administered on a daily basis since every other day regimens cannot maintain disease control. Doses of corticosteroids used in lupus are higher than in other diseases; when doses greater than 1 mg/day per day are required, patients receive intravenous methylprednisolone (Solu-Medrol) as pulse therapy (usually 250 mg to 1 g/day over several days). Such doses of corticosteroids help to control severe complications of lupus, including proliferative nephritis. DMARDs commonly in use are antimalarials such as plaquenil and immunosuppressants (e.g. methotrexate and azathioprine). Anti-BlyS antibodies (Benlysta™, Human Genome Science, Inc) was approved and can be used as a DMARD. Hydroxychloroquine is an antimalarial used for constitutional, cutaneous, and articular manifestations. Hydroxychloroquine has relatively few side effects, and there is evidence that it improves survival among people who have SLE. Cyclophosphamide is used for severe glomerulonephritis or other organ-damaging complications. Numerous new immunosuppressive drugs are being actively tested for SLE. Rather than suppressing the immune system nonspecifically, as corticosteroids do, they target the responses of individual immune cells; analgesics, such as indomethacin and diclofenac, may be used if over-the-counter drugs (mainly nonsteroidal anti-inflammatory drugs) do not provide effective relief. Intravenous immunoglobulins may be used to control SLE with organ involvement, or vasculitis. Due to the variety of symptoms and organ system involvement with SLE, its severity in an individual must be assessed in order to successfully treat SLE. Mild or remittent disease can sometimes be safely left untreated.

Treatment with an Anti-TLR3 Antibody and Corticosteroid Combination

The combination therapy can be used for the treatment of established lupus, or to reduce or abort a lupus flare, thereby leading to an improvement of the patient's health and comfort. In one embodiment, the subject is treated with an anti-TLR3 agent in combination with an orally administered, injected or infused corticosteroid, e.g. prednisone, for example at a dose of 40 mg per day, greater than 40 mg per day, or optionally less than 40 mg per day of prednisone equivalent. In one embodiment of any of the methods or uses, the corticosteroid is administered at a dose 1 mg/kg or more of prednisone-equivalent, and optionally of less than 1 mg/kg per day of prednisone equivalent. In one embodiment of any of the methods or uses, the corticosteroid is administered at a dose 0.5 mg/kg or more of prednisone-equivalent, and optionally of less than 0.5 mg/kg per day of prednisone equivalent. In one embodiment of any of the methods or uses, the corticosteroid is administered at a dose of 11-40 mg per day of prednisone equivalent. In one embodiment of any of the methods or uses, the corticosteroid is administered at a dose of 10 mg per day or less, optionally less than 10 mg per day of prednisone equivalent. Optionally, the corticosteroid is administered daily or once every other day (e.g. at least 3 times per week) of prednisone equivalent. In one embodiment, the corticosteroid is administered at less than 10 mg/kg per day. In one embodiment, the corticosteroid is selected from prednisone, prednisolone and methylprednisolone and the corticosteroid is administered at a dose of 40 mg per day or less, optionally 10 mg per day or less of prednisone equivalent, by oral administration, or optionally by intra-muscular (IM) injection into the skin for discoid rashes, or direct injection into a joint.

When the anti-TLR3 agent is an antibody, the antibody can be injected or infused for example via subcutaneous, intravenous, intramuscular, intra-articular, intra-synovial, intrathecal or intralesional routes.

Sepsis

Sepsis (systemic inflammatory response syndrome or SIRS) is a serious medical condition that is characterized by a whole-body inflammatory state. In addition to symptoms related to the provoking infection, sepsis is characterized by presence of acute inflammation present throughout the entire body, and is, therefore, frequently associated with fever and elevated white blood cell count (leukocytosis) or low white blood cell count and lower-than-average temperature, and vomiting. SIRS is characterized by hemodynamic compromise and resultant metabolic derangement. Outward physical symptoms of this response frequently include a high heart rate, high respiratory rate, elevated WBC count and elevated or lowered body temperature. Sepsis is differentiated from SIRS by the presence of a known pathogen. For example SIRS and a positive blood culture for a pathogen indicate the presence of sepsis. Without a known infection, it's not possible to classify the above symptoms as sepsis, only SIRS. This immunological response causes widespread activation of acute-phase proteins, affecting the complement system and the coagulation pathways, which then cause damage to the vasculature as well as to the organs. Various neuroendocrine counter-regulatory systems are then activated as well, often compounding the problem. Even with immediate and aggressive treatment, this may progress to multiple organ dysfunction syndrome and eventually death.

The American College of Chest Physicians and the Society of Critical Care Medicine has established different levels of sepsis. Systemic inflammatory response syndrome (SIRS) is defined by the presence of two or more of the following findings: Body temperature <36° C. (97° F.) or >38° C. (100° F.) (hypothermia or fever), heart rate >90 beats per minute, Respiratory rate >20 breaths per minute or, on blood gas, a PaCO2 less than 32 mm Hg (4.3 kPa) (tachypnea or hypocapnia due to hyperventilation), White blood cell count <4,000 cells/mm3 or >12,000 cells/mm3 (<4×109 or >12×109 cells/L), or greater than 10% band forms (immature white blood cells). (leukopenia, leukocytosis, or bandemia). Sepsis is defined as SIRS in response to a confirmed infectious process. Infection can be suspected or proven, or a clinical syndrome pathognomonic for infection. Severe sepsis is defined as sepsis with organ dysfunction, hypoperfusion, or hypotension. Septic shock is defined as sepsis with refractory arterial hypotension or hypoperfusion abnormalities in spite of adequate fluid resuscitation.

Sepsis is usually treated in the intensive care unit with intravenous fluids and antibiotics. If fluid replacement is insufficient to maintain blood pressure, specific vasopressor medications can be used. Mechanical ventilation and dialysis may be needed to support the function of the lungs and kidneys, respectively. To guide therapy, a central venous catheter and an arterial catheter may be placed. Sepsis patients require preventive measures for deep vein thrombosis, stress ulcers and pressure ulcers, unless other conditions prevent this. Treatment with an anti-TLR3 antibody and corticosteroid combination

One aspect is to provide a composition which is able to treat a SIRS, a sepsis, a severe sepsis or a septic shock. In one embodiment, the subject is treated with an anti-TLR3 agent in combination with an injected or infused administered corticosteroid, e.g. prednisone, at high doses for example dosed at least 1 mg/kg per day. In one embodiment, the corticosteroid is administered at less than less than 1 mg/kg per day. When the anti-TLR3 agent is an antibody, the antibody can be injected or infused via subcutaneous, intravenous, intramuscular, intra-articular, or intrathecal route.

Other Autoimmune and Inflammatory Disorders

The anti-TLR3 antibodies can be used to treat any other suitable autoimmune and inflammatory disorders that are known to be potentially responsive to corticosteroids, for example other inflammatory rheumatisms (juvenile rheumatoid arthritis, Still's disease, ankylosing spondylitis and other spondyloarthritides), acute or chronic hepatitis for which corticosteroids are indicated), sarcoidosis and other interstitial lung diseases, such as idiopathic pulmonary fibrosis, non-specific interstitial pneumonitis and organizing pneumoniaprimary or secondary glomerulonephritis for which corticosteroids are indicated (including but not limited to refractory nephrotic syndrome in adults); myasthenia gravis; systemic lupus erythematosus (SLE); antiphospholipid syndrome, the Sjögren's syndrome, systemic sclerosis, polymyositis, dermatomyositis; inflammatory muscle disorders, the Sharp's syndrome; connective tissue disease; chronic or sub-acute cutaneous lupus; psoriasis; epidermolysis bullosa and refractory mucosal lesions of pemphigus vulgaris; severe refractory atopic dermatitis; rosacea; primary vasculitis including but not limited to “ANCA Associated Vasculitis” (AAV, e.g., vasculitis associated with autoantibodies: the anti-neutrophil cytoplasmic antibodies (ANCA); these AAV include microscopic polyangitis, the Churg-Strauss' syndrome and Wegener's granulomatosis), polyartertitis nodosa, the Goodpasture's syndrome, giant cell arteritis and polymyalgia rheumatica, Behçet's disease, Schönlein-Henoch purpura, Takayasu's arteritis, Kawasaki disease; vasculitis secondary to any inflammatory disease; idiopathic thrombocytopenic purpura and autoimmune hemolytic anemia; thrombotic thrombocytopenic purpura and micrioangiopathic hemolytic anemia; the hypereosinophilic syndrome; uveitis secondary to an inflammatory disease; and acute or chronic Graft versus Host Disease.

The methods and compositions herein can be used advantageously in the treatment or prevention of acute or chronic Graft versus Host Disease. The ant-TLR3 agent and corticosteroid can be administered in combination to a subject who has received or who is expected to receive a transplant, e.g. a stem cell transplant, a hematopoietic stem cell transplant, an organ transplant. In one embodiment, the subject is treated with an anti-TLR3 antibody in combination with an injected or infused administered corticosteroid, e.g. prednisone, at high doses for example dosed at least 1 mg/kg per day. In one embodiment, the corticosteroid is administered at less than less than 1 mg/kg per day. When the anti-TLR3 agent is an antibody, the antibody can be injected or infused via subcutaneous, intravenous, intramuscular, intra-articular or intrathecal route. In one embodiment, the ant-TLR3 agent and corticosteroid are administered prior to a subject prior to transplantation. In one embodiment, the ant-TLR3 agent and corticosteroid are administered prior to a subject after transplantation.

Therapeutic Agents

Any corticosteroid having sufficient therapeutic activity can be used. Examples include hydrocortisone (Cortisol), cortisone acetate, dexamethasone (hereinafter, “Dexamethasone”), prednisone, prednisolone, methylprednisolone, betamethasone, triamcinolone, beclometasone, Paramethasone, fluticasone, fludrocortisone acetate, deoxycorticosterone acetate (DOCA), Fluprednisolone, fluticasone propionate, budesonide, beclomethasone dipropionate, flunisolide and triamcinolone acetonide.

Any anti-TLR3 agent that inhibits the activity of TLR3 and having sufficient therapeutic activity can be used. Optionally the anti-TLR3 agents binds directly to human TLR3, advantageously the anti-TLR3 agent is an antagonistic antibody that specifically binds to and inhibits TLR3, e.g., the antibodies are antibodies blocking the signalling induced by a TLR3 ligand (e.g. dsRNA) through TLR3.

In one embodiment, the antibodies have high affinity (e.g. subnanomoloar bivalent affinity) for a human TLR3 polypeptide at an acidic pH, i.e. a pH of about 5.6, of less than 10⁻⁹ M, optionally less than 10⁻¹⁰M. In another embodiment, the antibodies have high affinity at a neutral pH, i.e. a pH of about 7.2, of less than 10⁻⁹ M, optionally less than 10⁻¹⁰M. In another embodiment, the antibodies have high affinity at an acidic pH, i.e. a pH of about 5.6, and at a neutral pH, i.e. a pH of about 7.2, of less than 10⁻⁹ M, optionally less than 10⁻¹⁰M. In one embodiment, the antibodies are able to inhibit TLR3 signalling in the presence of a TLR3 ligand, i.e. dsRNA (polyAU, polyIC).

Producing Anti-TLR3 Antibodies

The antibodies suitable for use herein specifically bind TLR3. Particularly active antibodies furthermore bind TLR3 under acidic conditions corresponding to that encountered in an acidified endosomal compartment. Antibodies are furthermore capable of inhibiting the TLR3 signalling. In one embodiment, provided are methods of using an antibody that binds human TLR3, and competes for binding to human TLR3 with monoclonal antibody 31C3, 34A3, 11E1, 7G11, 31F6, 32C4 or 37B7 or mAb 15. Antibody 31C3 is produced by the cell deposited as 31C3.1 with the Collection Nationale de Culture de Microorganismes (CNCM), Institut Pasteur, 25 rue de Docteur Roux, F-75724 Paris on 3 Jul. 2009, under the number CNCM 1-4186. Antibody 29H3 is produced by the cell deposited as 29H3.7 with the Collection Nationale de Culture de Microorganismes (CNCM), Institut Pasteur, 25 rue de Docteur Roux, F-75724 Paris on 3 Jul. 2009, under the number CNCM 1-4187. Antibody mAb 15 (including all variants thereof, e.g. mAb 15EVQ) is described in PCT publication no. WO2010/127113, the disclosure of which is incorporated herein by reference in its entirety. Amino acid sequence of antibodies 11E1, 7G11, 31F6, 32C4 or 37B7 are disclosed herein.

“TLR3”, “TLR3 polypeptide” and “TLR3 receptor”, used interchangeably, are used herein to refer to Toll-Like Receptor 3, a member of the Toll-like receptor (TLRs) family. The amino acid sequence of human TLR3 is shown in SEQ ID NO: 1, below (NCBI accession number NP_003256, the disclosure of which is incorporated herein by reference). The human TLR3 mRNA sequence is described in NCBI accession number NM_003265. Human TLR3 sequences are also described in PCT patent publication no. WO 98/50547, the disclosure of which is incorporated herein by reference.

Human TLR3:
(SEQ ID NO: 1)
MRQTLPCIYF WGGLLPFGML CASSTTKCTV SHEVADCSHL
KLTQVPDDLP TNITVLNLTH NQLRRLPAAN FTRYSQLTSL
DVGFNTISKL EPELCQKLPM LKVLNLQHNE LSQLSDKTFA
FCTNLTELHL MSNSIQKIKN NPFVKQKNLI TLDLSHNGLS
STKLGTQVQL ENLQELLLSN NKIQALKSEE LDIFANSSLK
KLELSSNQIK EFSPGCFHAI GRLFGLFLNN VQLGPSLTEK
LCLELANTSI RNLSLSNSQL STTSNTTFLG LKWTNLTMLD
LSYNNLNVVG NDSFAWLPQL EYFFLEYNNI QHLFSHSLHG
LFNVRYLNLK RSFTKQSISL ASLPKIDDFS FQWLKCLEHL
NMEDNDIPGI KSNMFTGLIN LKYLSLSNSF TSLRTLTNET
FVSLAHSPLH ILNLTKNKIS KIESDAFSWL GHLEVLDLGL
NEIGQELTGQ EWRGLENIFE IYLSYNKYLQ LTRNSFALVP
SLQRLMLRRV ALKNVDSSPS PFQPLRNLTI LDLSNNNIAN
INDDMLEGLE KLEILDLQHN NLARLWKHAN PGGPIYFLKG
LSHLHILNLE SNGFDEIPVE VFKDLFELKI IDLGLNNLNT
LPASVFNNQV SLKSLNLQKN LITSVEKKVF GPAFRNLTEL
DMRFNPFDCT CESIAWFVNW INETHTNIPE LSSHYLCNTP
PHYHGFPVRL FDTSSCKDSA PFELFFMINT SILLIFIFIV
LLIHFEGWRI SFYWNVSVHR VLGFKEIDRQ TEQFEYAAYI
IHAYKDKDWV WEHFSSMEKE DQSLKFCLEE RDFEAGVFEL
EAIVNSIKRS RKIIFVITHH LLKDPLCKRF KVHHAVQQAI
EQNLDSIILV FLEEIPDYKL NHALCLRRGM FKSHCILNWP
VQKERIGAFR HKLQVALGSK NSVH

In one aspect, the anti-TLR3 antibody competes with monoclonal antibody 31C3, 34A3, 11E1, 7G11, 31F6, 32C4, 37B7 or mAb 15, and recognizes, binds to, or has immunospecificity for substantially or essentially the same, or the same, epitope or “epitopic site” on a TLR3 molecule as said monoclonal antibody 31C3, 34A3, 11E1, 7G11, 31F6, 32C4, 37B7 or mAb 15. In other embodiments, the monoclonal antibody consists of, or is a derivative or fragment of, antibody 31C3, 34A3, 11E1, 7G11, 31F6, 32C4, 37B7 or mAb 15 (e.g. mAb 15 EVQ).

It will be appreciated that, while exemplary antibodies bind to the same epitope as antibody 31C3, 34A3, 11E1, 7G11, 31F6, 32C4, 37B7 or mAb 15, the present antibodies can recognize and be raised against any part of the TLR3 polypeptide. For example, any fragment of TLR3, optionally but not exclusively human TLR3, or any combination of TLR3 fragments, can be used as immunogens to raise antibodies, and the antibodies of the invention can recognize epitopes at any location within the TLR3 polypeptide, so long as they can do so on TLR3 expressing cells such as MdDC or MoDC as described herein and inhibit TLR3 signalling.

The antibodies may be produced by a variety of techniques known in the art. Typically, they are produced by immunization of a non-human animal, e.g., a mouse, with an immunogen comprising a TLR3 polypeptide, e.g., a human TLR3 polypeptide. The TLR3 polypeptide may comprise the full length sequence of a human TLR3 polypeptide, or a fragment or derivative thereof, typically an immunogenic fragment, i.e., a portion of the polypeptide comprising an epitope exposed on the surface of cells expressing a TLR3 polypeptide, e.g., the epitope recognized by the 31C3, 34A3, 11E1, 7G11, 31F6, 32C4, 37B7 or mAb 15 antibody. Such fragments typically contain at least about 7 consecutive amino acids of the mature polypeptide sequence, or at least about 10 consecutive amino acids thereof. Fragments typically are essentially derived from the extra-cellular domain of the receptor. In one embodiment, the immunogen comprises a wild-type human TLR3 polypeptide in a lipid membrane, typically at the surface of a cell. In a specific embodiment, the immunogen comprises intact cells, particularly intact human cells, optionally treated or lysed. In another embodiment, the polypeptide is a recombinant TLR3 polypeptide.

The step of immunizing a non-human mammal with an antigen may be carried out in any manner well known in the art for stimulating the production of antibodies in a mouse (see, for example, E. Harlow and D. Lane, Antibodies: A Laboratory Manual., Cold Spring Harbor Laboratory Press, Cold Spring Harbor, N.Y. (1988), the entire disclosure of which is herein incorporated by reference). The immunogen is suspended or dissolved in a buffer, optionally with an adjuvant, such as complete or incomplete Freund's adjuvant. Methods for determining the amount of immunogen, types of buffers and amounts of adjuvant are well known to those of skill in the art and are not limiting in any way. These parameters may be different for different immunogens, but are easily elucidated.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with an adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be used as long as they result in the production of B cells expressing an antibody directed to the antigen used in immunization.

For polyclonal antibody preparation, serum is obtained from an immunized non-human animal and the antibodies present therein isolated by well-known techniques. The serum may be affinity purified using any of the immunogens set forth above linked to a solid support so as to obtain antibodies that react with TLR3 polypeptides.

In an alternate embodiment, lymphocytes from a non-immunized non-human mammal are isolated, grown in vitro, and then exposed to the immunogen in cell culture. The lymphocytes are then harvested and the fusion step described below is carried out.

For exemplary monoclonal antibodies, the next step is the isolation of splenocytes from the immunized non-human mammal and the subsequent fusion of those splenocytes with an immortalized cell in order to form an antibody-producing hybridoma. The isolation of splenocytes from a non-human mammal is well-known in the art and typically involves removing the spleen from an anesthetized non-human mammal, cutting it into small pieces and squeezing the splenocytes from the splenic capsule through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the lymphocytes can be fused to an immortal cell line. This is typically a mouse myeloma cell line, although many other immortal cell lines useful for creating hybridomas are known in the art. Murine myeloma lines include, but are not limited to, those derived from MOPC-21 and MPC-11 mouse tumors available from the Salk Institute Cell Distribution Center, San Diego, U.S.A, X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Hybridomas are typically grown on a feeder layer of macrophages. The macrophages can be from littermates of the non-human mammal used to isolate splenocytes and are typically primed with incomplete Freund's adjuvant or the like several days before plating the hybridomas. Fusion methods are described in Goding, “Monoclonal Antibodies: Principles and Practice,” pp. 59-103 (Academic Press, 1986), the disclosure of which is herein incorporated by reference.

The cells are allowed to grow in the selection media for sufficient time for colony formation and antibody production. This is usually between about 7 and about 14 days.

The hybridoma colonies are then assayed for the production of antibodies that specifically bind to TLR3 polypeptide gene products, optionally the epitope specifically recognized by antibody 31C3, 34A3, 11E1, 7G11, 31F6, 32C4, 37B7 or mAb 15. The assay is typically a colorimetric ELISA-type assay, although any assay may be employed that can be adapted to the wells that the hybridomas are grown in. Other assays include radioimmunoassays or fluorescence activated cell sorting. The wells positive for the desired antibody production are examined to determine if one or more distinct colonies are present. If more than one colony is present, the cells may be re-cloned and grown to ensure that only a single cell has given rise to the colony producing the desired antibody. Typically, the antibodies will also be tested for the ability to bind to TLR3 polypeptides, e.g., TLR3-expressing cells, in paraffin-embedded tissue sections, as described below.

Hybridomas that are confirmed to produce a monoclonal antibody can be grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

Positive wells with a single apparent colony are typically re-cloned and re-assayed to insure only one monoclonal antibody is being detected and produced.

Antibodies may also be produced by selection of combinatorial libraries of immunoglobulins, as disclosed for instance in (Ward et al. Nature, 341 (1989) p. 544, the entire disclosure of which is herein incorporated by reference).

The identification of one or more antibodies that bind(s) to TLR3, particularly substantially or essentially the same epitope as monoclonal antibody 31C3, 34A3, 11E1, 7G11, 31F6, 32C4, 37B7 or mAb 15, can be readily determined using any one of a variety of immunological screening assays in which antibody competition can be assessed. Many such assays are routinely practiced and are well known in the art (see, e. g., U.S. Pat. No. 5,660,827, issued Aug. 26, 1997, which is specifically incorporated herein by reference). It will be understood that actually determining the epitope to which an antibody described herein binds is not in any way required to identify an antibody that binds to the same or substantially the same epitope as the monoclonal antibody described herein. Methods for determining competition and for determining epitopes on TLR3 are described in PCT patent publication WO2012/095432 (Innate Pharma), which is specifically incorporated herein by reference.

The antibodies inhibit the activation of TLR3-expressing cells, e.g. they can inhibit the TLR3 signalling pathway, with or without blocking the binding (or by partially blocking binding) to TLR3 of natural or endogenous ligands such as dsRNA; optionally they may block the ability of TLR3 protein to form homodimers in the presence of a TLR3 ligand, thus blocking the initiation a signalling cascade. These antibodies are thus referred to as “neutralizing” or “inhibitory” or “blocking” antibodies. A range of cellular assays can be used to assess the ability of the antibodies to modulate TLR3 signalling. Any of a large number of assays, including molecular, cell-based, and animal-based models can be used to assess the ability of anti-TLR3 antibodies to modulate TLR3-expressing cell activity. For example, cell-based assays can be used in which cells expressing TLR3 are exposed to dsRNA, viral dsRNA, polyIC, or poly AU, or another TLR3 ligand and the ability of the antibody to disrupt the binding of the ligand or the stimulation of the receptor (as determined, e.g., by examining any of the TLR3 cell activities addressed herein, such as interferon expression, NFkB activity, NK cell activation, etc.) is assessed. The TLR3 ligand used in the assays may be in any suitable form, including but not limited to as a purified ligand composition, in a mixture with non-TLR3 ligands, in a naturally occurring composition, in a cell or on the surface of a cell, or secreted by a cell (e.g. a cell that produces ligand is used in the assay), in solution or on a solid support. Assays for assessing TLR3 inhibition are described in PCT patent publication WO2012/095432 (Innate Pharma), which is specifically incorporated herein by reference.

Antibody CDR Sequences

Exemplary anti-TLR3 antibodies include antibodies comprising the CDRs 1, 2 and 3 of the heavy and light chains of anti-TLR3 antibodies 31C3, 34A3, 11E1, 7G11, 31F6, 32C4, 37B7 or mAb 15, e.g, the HCDRs 1, 2 and 3 having the amino acid of the respective SEQ ID NO: listed below in Table A and the LCDRs 1, 2 and 3 having the amino acid of the respective SEQ ID NO: listed below in Table B.

Exemplary anti-TLR3 antibodies also include antibodies comprising the CDRs 1, 2 and 3 of the heavy and light variable chains of the anti-TLR3 antibodies 31C3, 34A3, 11E1, 7G11, 31F6, 32C4, 37B7 or mAb 15 having the respective VH and VL amino acid sequences shown in SEQ ID NOS: 65-80, respectively. The CDRs or antibodies may optionally be characterized as having an amino acid sequence that shares at least 50%, 60%, 70%, 80%, 85%, 90% or 95% sequence identity with the particular VH, VL, CDR sequences or set of CDR sequences.

In any anti-TLR3 antibodies, e.g., 31C3, 34A3, 11E1, 7G11, 31F6, 32C4, 37B7 or mAb 15, the specified variable region and CDR sequences may comprise conservative sequence modifications. Conservative sequence modifications refer to amino acid modifications that do not significantly affect or alter the binding characteristics of the antibody containing the amino acid sequence. Such conservative modifications include amino acid substitutions, additions and deletions. Modifications can be introduced into an antibody by standard techniques known in the art, such as site-directed mutagenesis and PCR-mediated mutagenesis. Conservative amino acid substitutions are typically those in which an amino acid residue is replaced with an amino acid residue having a side chain with similar physicochemical properties. Specified variable region and CDR sequences may comprise one, two, three, four or more amino acid insertions, deletions or substitutions. Where substitutions are made, exemplary substitutions will be conservative modifications. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g. threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for retained function (i.e., the properties set forth herein) using the assays described herein.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Exemplary methods for determining identity are designed to give the largest match between the sequences tested. Methods of determining identity are described in publicly available computer programs. A computer based method for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

The sequences of the CDRs, according to AbM (Oxford Molecular's AbM antibody modelling software definition), Kabat and Chothia definitions systems, have been summarized in Tables A and B below. The amino acids sequences described herein are numbered according to Abm, Kabat and Chothia numbering systems. While any suitable numbering system may be used to designated CDR regions, in the absence of any other indication, the numbering used herein is Abm. Such numbering has been established using the following indications: CDR-L1: Start: approx. residue 24, residue before: always a Cys, residue after: always a Trp (typically Trp-Tyr-Gln, but also, Trp-Leu-Gln, Trp-Phe-Gln, Trp-Tyr-Leu), length: 10 to 17 residues; CDR-L2: Start: always 16 residues after the end of L1, Residues before: generally Ile-Tyr (but also, Val-Tyr, Ile-Lys, Ile-Phe), Length: always 7 residues; CDR-L3, Start: always 33 residues after end of L2, Residue before: always Cys, Residues after: always Phe-Gly-Xaa-Gly, Length: 7 to 11 residues; CDR-H1, Start: approx residue 26 (always 4 after a Cys) (Chothia/AbM definition, the Kabat definition starts 5 residues later), Residues before: always Cys-Xaa-Xaa-Xaa, Residues after: always a Trp (typically Trp-Val, but also, Trp-Ile, Trp-Ala), Length: 10 to 12 residues (AbM definition, Chothia definition excludes the last 4 residues); CDR-H2, Start: always 15 residues after the end of Kabat/AbM definition of CDR-H1, Residues before: typically Leu-Glu-Trp-Ile-Gly (but a number of variations, Residues after Lys/Arg-Leu/Ile/Val/Phe/Thr/Ala-Thr/Ser/Ile/Ala), Length: Kabat definition 16 to 19 residues; AbM (and Chothia) definition ends 7 residues earlier; CDR-H3, Start: always 33 residues after end of CDR-H2 (always 2 after a Cys), Residues before: always Cys-Xaa-Xaa (typically Cys-Ala-Arg), Residues after: always Trp-Gly-Xaa-Gly, Length: 3 to 25 residues.

The sequences of the variable chains of the antibodies are listed in Table c below.

TABLE A

| | CDR | HCDR1 | | HCDR2 | | HCDR3 | |
|---|---|---|---|---|---|---|---|
| | defini- | SEQ | | SEQ | | SEQ | |
| mAb | tion | ID | Sequence | ID | Sequence | ID | Sequence |
| 11E1 | Kabat | 2 | NYWMN | 5 | MIDPSDSETHYNQMFKD | 7 | GASSDYYYFDY |
| | Chotia | 3 | GYTFTN | 6 | MIDPSDSETH | | GASSDYYYFDY |
| | Abm | 4 | GYTFTNYWMN | | MIDPSDSETH | | GASSDYYYFDY |
| 31F6 | Kabat | 11 | YTYMH | 14 | RIDPANGDSIYGEDFKT | 16 | GEFDYFDY |
| | Chotia | 12 | GYNIRY | 15 | RIDPANGDSI | | GEFDYFDY |
| | Abm | 13 | GYNIRYTYMH | | RIDPANGDSI | | GEFDYFDY |
| 32C4 | Kabat | 20 | STYIY | 23 | RIDPANGNTIYAEKFKT | 25 | GDHGGYVMDA |
| | Chotia | 21 | GYNIWS | 24 | RIDPANGNTI | | GDHGGYVMDA |
| | Abm | 22 | GYNIWSTYIY | | RIDPANGNTI | | GDHGGYVMDA |
| 37B7 | Kabat | 29 | SNYMH | 32 | WIFPGDGDTNYNQKFNG | 34 | SDGDWYFDF |
| | Chotia | 30 | GYTFTS | 33 | WIFPGDGDTN | | SDGDWYFDF |
| | Abm | 31 | GYTFTSNYMH | | WIFPGDGDTN | | SDGDWYFDF |
| 7G11 | Kabat | 38 | YTYMH | 41 | RIDPANGNTIYGEKFKN | 43 | GEFDYFDH |
| | Chotia | 39 | GYNITY | 42 | RIDPANGNTI | | GEFDYFDH |
| | Abm | 40 | GYNITYTYMH | | RIDPANGNTI | | GEFDYFDH |
| 31C3 | Kabat | 47 | AYYMH | 50 | RINPYNGATSYNRNFKD | 52 | SGGNTYFDY |
| | Chotia | 48 | GYSFTA | 51 | RINPYNGATS | | SGGNTYFDY |
| | Abm | 49 | GYSFTAYYMH | | RINPYNGATS | | SGGNTYFDY |
| 34A3 | Kabat | 56 | TYSIY | 59 | YIDPYNGDTSYNQKFKG | 61 | EGNYYGYFDY |
| | Chotia | 57 | GYVFTT | 60 | YIDPYNGDTS | | EGNYYGYFDY |
| | Abm | 58 | GYVFTTYSIY | | YIDPYNGDTS | | EGNYYGYFDY |

TABLE B

| | CDR | LCDR1 | | LCDR2 | | LCDR3 | |
|---|---|---|---|---|---|---|---|
| | defini- | SEQ | | SEQ | | SEQ | |
| mAb | tion | ID | Sequence | ID | Sequence | ID | Sequence |
| 11E1 | Abm, | 8 | ITSTDIHDDIN | 9 | EGNTLRP | 10 | LQSDNLPRT |
| | Chotia, | | | | | | |
| | Kabat | | | | | | |
| 31F6 | Abm, | 17 | LASEGISNDLA | 18 | AASRLQD | 19 | QQNYKYPLT |
| | Chotia, | | | | | | |
| | Kabat | | | | | | |
| 32C4 | Abm, | 26 | LASEDIYSYLA | 27 | AANRLED | 28 | LQGSEFPYT |
| | Chotia, | | | | | | |
| | Kabat | | | | | | |
| 37B7 | Abm, | 35 | QASEDIYSGLA | 36 | AASRLQD | 37 | QQGVKYPNT |
| | Chotia, | | | | | | |
| | Kabat | | | | | | |
| 7G11 | Abm, | 44 | LASEDISNDLA | 45 | AASRLED | 46 | QQSYKYPVT |
| | Chotia, | | | | | | |
| | Kabat | | | | | | |
| 31C3 | Abm, | 53 | RASENIYSSL | 54 | NAKTLAE | 55 | QHHYGTPPT |
| | Chotia, | | | | | | |
| | Kabat | | | | | | |
| 34A3 | Abm, | 62 | SASSSVSYMF | 63 | LTSNLA | 64 | QQWTGNPPT |
| | Chotia, | | | | | | |
| | Kabat | | | | | | |

TABLE C

| | SEQ | |
|---|---|---|
| Antibody | ID | |
| portion | NO: | Variable region amino acid sequence |
| 11E1 VH | 65 | Q V Q L Q Q P G A E L V R P G A S V K L S C K A S G |
| | | Y T F T N Y W M N W V K Q R P G Q G L E W I G M I D |
| | | P S D S E T H Y N Q M F K D K A T L T V D K S S S T |
| | | A Y M Q L S S L T S E D S A V Y Y C A R G G A S S D |
| | | Y Y Y F D Y W G Q G T T L T V S S A S T K |
| 11E1 VL | 66 | E T T V T Q S P A S L S M A I G E K V T I R C I T S |
| | | T D I H D D I N W Y Q Q K P G E P P K L L I S E G N |
| | | T L R P G V P S R F S S S G Y G T D F V F T I E N M |
| | | L S E D V A D Y Y C L Q S D N L P R T F G G G T K L |
| | | E I K R T |
| 31F6 VH | 67 | E V Q L Q Q S G A E L G K P G T S V K L S C K V S G |
| | | Y N I R Y T Y M H W V N Q R P G K G L E W I G R I D |
| | | P A N G D S I Y G E D F K T K A T L T A D T S S N T |
| | | A Y M Q L S Q L K S D D T A I Y F C A M G E F D Y F |
| | | D Y W G Q G V M V T V S S A S T K |
| 31F6 VL | 68 | D I Q M T Q S P P S L S A S L G E T V S I E C L A S |
| | | E G I S N D L A W Y Q Q R S G K S P Q L L I Y A A S |
| | | R L Q D G V P S R F S G S G S G T R Y S L K I S G M |
| | | Q P E D E A D Y F C Q Q N Y K Y P L T F G S G T K L |
| | | E I K |
| 32C4 VH | 69 | E V Q L Q Q Y G A E L G K P G T S V K L S C K V S G |
| | | Y N I W S T Y I Y W V N Q R P G K G L E W I G R I D |
| | | P A N G N T I Y A E K F K T K A T L T A D T S S N T |
| | | A Y M Q L S Q L K S D D T A I Y F C A M G D H G G Y |
| | | V M D A W G Q G A S V T V S S |
| 32C4 VL | 70 | D I Q M T Q S P G S L S A S L G E T V S I E C L A S |
| | | E D I Y S Y L A W Y Q Q K P G K S P Q L L I Y A A N |
| | | R L E D G V P S R F S G S G S G T Q Y S L K I S G M |
| | | Q P E D E G D Y F C L Q G S E F P Y T F G T G T K L |
| | | E L K |
| 37B7 VH | 71 | Q V Q L Q Q S G T E L V K P G S S V K I S C K A S G |
| | | Y T F T S N Y M H W I R Q L P G N G L E W I G W I F |
| | | P G D G D T N Y N Q K F N G K A T L T A D K S S S T |
| | | A Y M Q L S S L T S E D Y A V Y F C A R S D G D W Y |
| | | F D F W G P G T M V T V S S |
| 37B7 VL | 72 | D I Q M T Q S P G S L S A S L G E T V T I Q C Q A S |
| | | E D I Y S G L A W Y Q Q K P R K S P Q L L I S A A S |
| | | R L Q D G V P S R F S G S G S G T Q Y S L K I S S M |
| | | Q T E D E G V Y F C Q Q G V K Y P N T F G P G T K L |
| | | E L K |
| 7G11 VH | 73 | E V Q L Q Q Y G A E L G K P G T S V K L S C K V S G |
| | | Y N I T Y T Y M H W V N Q R P G K G L E W I G R I D |
| | | P A N G N T I Y G E K F K N K A T L T A D T S S N T |
| | | A Y M Q L S Q L K S D D T A I Y F C A M G E F D Y F |
| | | D H W G Q G V M V T V S S |
| 7G11 VL | 74 | D I Q M T Q S P A S L S A S L G E T V S I E C L A S |
| | | E D I S N D L A W Y Q Q K S G K S P Q V L I Y A A S |
| | | R L E D G V P S R F S G S G S G T R Y S L K I S G M |
| | | Q P E D E A D Y F C Q Q S Y K Y P V T F G S G T K L |
| | | E I K |
| 31C3 VH | 75 | MGWSWIFLFL LSGTAGVLSE VQLQQSGPEL VKPGASVKIS |
| | | CKPSGYSFTA YYMHWVKQSH VKSLEWIGRI NPYNGATSYN |
| | | RNFKDKASLT VDKSSSTAYM ELHSLTSEDS AVYYCARSGG |
| | | NTYFDYWGQG TTLTVS |
| 31C3 VL | 76 | MSVPTQVLGL LLLWLTGARC DIQMTQSPAS LSASVGETVT |
| | | ITCRASENIY SSLAWYQQKQ GKSPQLLVYN AKTLAEGVPS |
| | | RFSGSGSGTQ F/SSLKINSLQP EDFGTYYCQH HYGTPPTFGG |
| | | GTKLEIK |
| 34A3 VH | 77 | MEWRWIFLFL LSGTTGVHSE IQLQQSGPEL VKPGASVKVS |
| | | CKASGYVFTT YSIYWVKQSH GKSLEWIGYI DPYNGDTSYN |
| | | QKFKGKATLT VDKSSSTAYM HLNSLTSEDS TVYYCAREGN |
| | | YYGYFDYWGQ GTTLTVS |
| 34A3 VL | 78 | MDFQVQIFSF LLMSASVIMS RGQIVLTQSP ALMSASPGEK |
| | | VTMTCSASSSV SYMFWYQQKP RSSPKPWIYL TSNLASGVPA |
| | | RFSGSGSGTS YSLTISSMEA EDAATYYCQQ WTGNPPTFGG |
| | | GTKLEIK |

The amino acid sequences of the heavy and light chains of antibody mAb 15 are shown below (without leader sequences, see also WO2010/051470)

mAb 15 EVQ heavy chain (IgG4):
(SEQ ID NO: 79)
EVQLVQSGAEVKKPGESLKISCKGSGYSFTNYWVGWVRQMPGKGLEW
MGFIDPSDSYTNYAPSFQGQVTISADKSISTAYLQWSSLKASDTAMY
YCARELYQGYMDTFDSWGQGTLVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEA
AGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPS
DIAVEWESNGQPENNYKTTPPVLDSDGSFFLYSRLTVDKSRWQEGNV
FSCSVMHEALHNHYTQKSLSLSLGK
mAb 15 light chain:
(SEQ ID NO: 80)
DIQMTQSPSSLSASVGDRVTITCRASQSIGLYLAWYQQKPGKAPKLLI
YAASSLQSGVPSRFSGSGSGTDFTLTISSLQPEDFATYYCQQGNTLSY
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC

Fragments and derivatives of antibodies (which are encompassed by the term “antibody” or “antibodies” as used in this application, unless otherwise stated or clearly contradicted by context), optionally a 31C3, 34A3, 11E1, 7G11, 31F6, 32C4, 37B7 or mAb 15-like antibody, can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. Included, inter alia, are a nanobody, domain antibody, single domain antibody or a “dAb”.

Thus, according to another embodiment, the antibody, optionally a 31C3, 34A3, 11E1, 7G11, 31F6, 32C4, 37B7 or mAb 15-like antibody, is humanized. “Humanized” forms of antibodies are specific chimeric immunoglobulins, immunoglobulin chains or fragments thereof (such as Fv, Fab, Fab′, F (ab′) 2, or other antigen-binding subsequences of antibodies) which contain minimal sequence derived from the murine immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody.

In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al, Nature, 332, pp. 323 (1988); Presta, Curr. Op. Struct. Biol., 2, pp. 593 (1992); Verhoeyen et Science, 239, pp. 1534; and U.S. Pat. No. 4,816,567, the entire disclosures of which are herein incorporated by reference.) Methods for humanizing the antibodies are well known in the art.

The choice of human variable domains, both light and heavy, to be used in making the humanized antibodies is very important to reduce antigenicity. According to the so-called “best-fit” method, the sequence of the variable domain of an antibody is screened against the entire library of known human variable-domain sequences. The human sequence which is closest to that of the mouse is then accepted as the human framework (FR) for the humanized antibody (Sims et al., J. Immunol. 151, pp. 2296 (1993); Chothia and Lesk, J. Mol. 196, 1987, pp. 901). Another method uses a particular framework from the consensus sequence of all human antibodies of a particular subgroup of light or heavy chains. The same framework can be used for several different humanized antibodies (Carter et al., PNAS 89, pp. 4285 (1992); Presta et al., J. Immunol., 151, p. 2623 (1993)).

It is further important that antibodies be humanized with retention of high affinity for TLR3 receptors and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen (s), is achieved. In general, the CDR residues are directly and most substantially involved in influencing antigen binding.

Another method of making “humanized” monoclonal antibodies is to use a XenoMouse (Abgenix, Fremont, Calif.) as the mouse used for immunization. A XenoMouse is a murine host according that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference.

Human antibodies may also be produced according to various other techniques, such as by using, for immunization, other transgenic animals that have been engineered to express a human antibody repertoire (Jakobovitz et Nature 362 (1993) 255), or by selection of antibody repertoires using phage display methods. Such techniques are known to the skilled person and can be implemented starting from monoclonal antibodies as disclosed in the present application.

Therapeutic formulations of the antibodies are prepared for storage by mixing the antagonist having the desired degree of purity with optional pharmaceutically acceptable carriers, excipients, or stabilizers in the form of lyophilized formulations or aqueous solutions. For general information concerning formulations, see, e.g., Gilman et al. (eds.), The Pharmacological Bases of Therapeutics, 8^{th} Ed. (Pergamon Press, 1990); Gennaro (ed.), Remington's Pharmaceutical Sciences, 18^{th} Edition (Mack Publishing Co., Easton, Pa., 1990); Avis et al. (eds.), Pharmaceutical Dosage Forms: Parenteral Medications (Dekker, New York, 1993); Lieberman et al. (eds.), Pharmaceutical Dosage Forms: Tablets (Dekker, New York, 1990); Lieberman et al. (eds.) Pharmaceutical Dosage Forms: Disperse Systems (Dekker, New York, 1990); and Walters (ed.), Dermatological and Transdermal Formulations (Drugs and the Pharmaceutical Sciences), Vol 119 (Dekker, New York, 2002).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as ethylenediaminetetraacetic acid (EDTA); sugars such as sucrose, mannitol, trehalose, or sorbitol; salt-forming counter-ions such as sodium; metal complexes (e.g., Zn-protein complexes); and/or non-ionic surfactants such as TWEEN™, PLURONICS™, or PEG.

Exemplary antibody formulations are described for instance in WO 1998/56418, which describes a liquid multidose formulation for an anti-CD20 antibody, comprising 40 mg/mL rituximab, 25 mM acetate, 150 mM trehalose, 0.9% benzyl alcohol, and 0.02% polysorbate20™ at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL polysorbate80™, and Sterile Water for Injection, pH 6.5.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

The formulations to be used for in vivo administration must be sterile. This is readily accomplished by filtration through sterile filtration membranes. Pharmaceutically acceptable carriers that may be used in these compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The antibodies may be employed in a method of modulating, e.g. inhibiting, the activity of TLR3-expressing cells in a patient. This method comprises the step of contacting said composition with said patient. Such method will be useful for both prophylaxis and therapeutic purposes.

Further aspects and advantages will be disclosed in the following experimental section, which should be regarded as illustrative and not limiting the scope of this application.

### EXAMPLES

### Materials and Methods

Tumor Cell lines: A375 malignant melanoma tumor cell lines (CRL-1619) and 293T Human Embryonic Kidney cells (#CRL-1573) are purchased from ATCC. Antibodies (antigen, supplier, reference): Anti-TLR3 antibody pAb, R&D Systems, ref. AF1487, anti-TLR3 antibody mAb clone TLR3.7 from eBioscience, anti-TLR3 mAb from lmgenex clone 40C1285. Instrumentation: FACSCalibur™ flow cytometer (BD Biosciences). PolyAU, also referred to as IPH3102, is an at least partially double stranded molecule made of polyadenylic acid(s) and polyuridylic acid(s), prepared as described in WO2009/130616 (Innate Pharma), the disclosure of which is incorporated herein by reference. PolyAU was a high molecular weight polyAU having an Mₙ (also referred to as “number average molecular weight” or “mean molecular weight”) above 2000 kD, a PI of 1.4-1.6, and thermal stability: 62.3-63.2° C., hyperchromicity of 53-60%.

Surface Plasmon Resonance (SPR)

(a) General Biacore T100 methods. SPR measurements were performed on a Biacore T100 apparatus (Biacore GE Healthcare) at 25° C. In all Biacore experiments HBS-EP+buffer (Biacore GE Healthcare) or 10 mM sodium acetate pH 5.6, 150 mM NaCl, 0.05% P20 served as running buffer and sensorgrams were analyzed with Biaevaluation 4.1 and Biacore T100 Evaluation software. Recombinant human and mouse TLR3 were purchased from R&D Systems.

(b) Protein immobilization. Recombinant TLR3 protein was immobilized covalently to carboxyl groups in the dextran layer of a Biacore Series 5 Sensor Chip CM5 (chip). The chip surface was activated with EDC/NHS (0.2M N-ethyl-N′-(3-dimethylaminopropyl) carbodiimidehydrochloride, 0.05M N-hydroxysuccinimide (Biacore GE Healthcare)). Proteins were diluted to 10 μg/ml in coupling buffer (10 mM sodium acetate, pH 5.6) and injected until the appropriate immobilization level was reached (i.e. approximately 2000 RU for binding experiments and 600 RU for affinity experiments). Deactivation of the remaining activated groups was performed using 100 mM ethanolamine pH 8 (Biacore GE Healthcare).

(c) Antibody binding analysis was run using HBS-EP+(neutral pH). Antibodies at a concentration of 10 μg/ml were injected for 2 min at a constant flow rate of 10 μl/min over the immobilized proteins and allowed to dissociate for 3 min before regeneration by a ten second injection of 10 mM NaOH, 500 mM NaCl regeneration buffer. Blank correction was performed on line by co-injecting the soluble antibodies onto the reference dextran flow cell.

(d) Competition assay in acidic buffer (pH 5.6). Flow rate is set to 10 μl/min, the first antibody at a concentration of 50 μg/ml (or polyAU at 100 μg/ml) was injected for 2 min, 3 times successively in order to saturate the rhTLR3 surface. The second antibody (or polyAU at 100 μg/ml when the polyAU was added after first antibody) is then injected for 2 min also at 50 μg/ml and allowed to dissociate for 3 min before regeneration by a 15 second injection of 10 mM NaOH, 500 mM NaCl regeneration buffer. Blank correction is also performed on line and the curve using the saturating antibody (or nucleic acid) followed by an injection of buffer subtracted to remove the signal due to the dissociation of the first complex. The resulting signal is compared to that obtained by the injection of the second antibody directly onto the rhTLR3 surface.

Luciferase Reporter Assay.

A reporter gene assay using as promoter ISRE (IFN-stimulated response element) and as reporter gene and protein luciferase was set up. A 293T cell line (ATCC, #CRL-1573) was stably transfected with pISRE-luc plasmid (#219089—Stratagene), further selected by cloning as inducing optimal response to IFN-alpha stimulation and referred to as control 293T-ISRE. This cell line was further stably transfected with pUNO-humanTLR3 plasmid (#puno-htlr3—InVivogen) or pUNO-mouseTLR3 plasmid (#puno-mtlr3—InVivogen) and referred to as 293T-TLR3-ISRE and 293T-mTLR3-ISRE respectively. On day 0, cells are seeded at 4×105 cells/mL in complete culture medium in 96-well culture plate (100 μl/well). Cells are first incubated at 37° C. for 20 hours, then 50 μL of medium are discarded and cells are activated with 100 μL/well final of increasing amounts of polyAU together with various concentrations of anti-TLR3 antibodies. Cells incubated with fresh medium will be used as background luciferase activity. Cells are incubated at 37° C. for 6 hours. 100 μL of freshly thawed Steady Glo (Promega) are added to each well, plates were incubated 10 min at RT in the dark and the light emitted in each well is quantified as Count Per Second (CPS) on a gamma-counter (TopCount) apparatus.

MdDC Secretion and Expression Inhibition In Vitro Tests.

Myeloid DC (MdDC) are obtained from PBMC by isolating PBMC from normal healthy human donors. Monocytes are purified from PBMC using positive selection with human CD14 microbeads (Miltenyi Biotech) following general instructions. Monocytes are further derived into DC (MdDC) by 5-6 days incubation in human GM-CSF (Leucomax, SP) and human IL-4 (R&D Systems) at respectively 200 ng/ml and 20 ng/ml.

The resulting MdDC are then seeded at 10⁶ cells/ml, in duplicate, in flat bottom 96-well plates. Cells are activated for 20 hours in a final volume of 200 μl, together with the anti-TLR3 antibodies at the indicated concentrations. Increasing amounts of polyAU are added to the wells to obtain a dose effect read-out at the dose and timepoint as indicated.

Supernatants are collected after 20 h of stimulation, frozen at −20° C., and further assayed for IL-6 and IP-10 using Enzyme-linked immunosorbent assay. Cells are then harvested, stained for activation markers CD86 (with a.huCD86 mouse, IgG1, FITC, BD Biosciences, ref 555657), with detection using FACSCanto™ flow cytometer (BD Biosciences).

### Example 1

### Generation of TLR3-Specific Monoclonal Rat Anti-Mouse Antibodies

Primary screen. To obtain anti-TLR3 antibodies, LOU/c rats were immunized with a recombinant His-tagged mouse TLR3, carrier free extracellular domain recombinant protein (R&D systems, #3005-TR) and recombinant His-tagged human TLR3, carrier free extracellular domain recombinant protein (R&D systems, #1487-TR). Rats received, on day 0, one primo-immunisation with an emulsion of 50 μg of mouse TLR3+50 μg of human TLR3 diluted in PBS and Complete Freund Adjuvant, intraperitoneally, a 2^{nd} immunization on day 14 with an emulsion of 50 μg of mouse TLR3+50 μg of human TLR3 diluted in PBS and Incomplete Freund Adjuvant, intraperitoneally, and one boost with 25 μg of mouse TLR3+25 μg of human TLR3 diluted in PBS, intravenously. Immune spleen cells were fused with X63.Ag8.653 immortalized B cells, and cultured in the presence of irradiated spleen cells.

40 culture plates were obtained and evaluated in a first screen for mouse TLR3 binding using an ELISA developed for detection of binding to TLR3. Briefly, His-tagged recombinant mouse TLR3 protein (R&D systems, #1487-TR-050) was coated on Ni-NTA 96-wells plates (Qiagen). Supernatant (SN) from hybridoma culture plates and incubated in TLR3-plates, and the presence of TLR3 binding Ig was revealed with goat anti-mouse F(ab) IgG-HRP.

Secondary screen: selection of hybridomas of interest. 181 supernatants were retained and tested in a further screen in an inhibition test on 293T-mTLR3 cells. Wells from supernatants having an inhibitory effect superior to 95% were selected for further cloning by limiting dilution.

Cloning of hybridomas of potential interest. 27 potentially interesting hybridomas selected from the initial screening were cloned by limiting dilution techniques in 96-wells plates, and 370 subclones were evaluated in a screen for mouse TLR3 binding using an ELISA as above. The 178 positive clones were tested in a further screen in an inhibition test on 293T-TLR3 cells as above. Among them was supernatant from well G7 from plate 28 (28G7).

### Example 2

### Reporter Assay

Antibodies were tested for inhibition of TLR3 signalling in a luciferase-based reporter gene activity (293T-TLR3-ISRE). Engagement of TLR3 receptor using TLR3-agonists such as poly (I:C) has been reported to activate the type-IFN pathway including the promoter ISRE (Wietek et al. J. Biol. Chem., 278(51), p 50923, 2003). Briefly, dsRNA TLR3 agonists were used to induce TLR3 signalling in the reporter assay in the presence of anti-TLR3 antibodies, and TLR3 signalling was assessed.

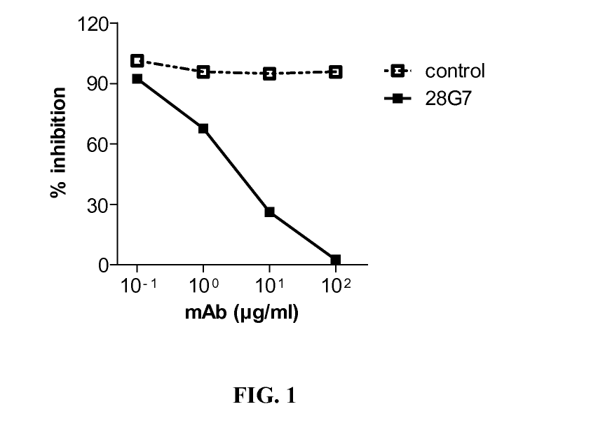

Rat anti-mouse TLR3 antibodies were assessed for their ability to inhibit TLR3 signalling in a luciferase based reporter gene activity (293T-mTLR3-ISRE). FIG. 1 shows the inhibition properties of increasing doses of the antibody 28G7 (black squares, full line), in comparison with a non-relevant control antibody (control, open squares, dashed line), the inhibition of the TLR3 signalling is dose dependent, with an IC50 of 2.6 μg/ml.

### Example 3

### TLR3 Modulation

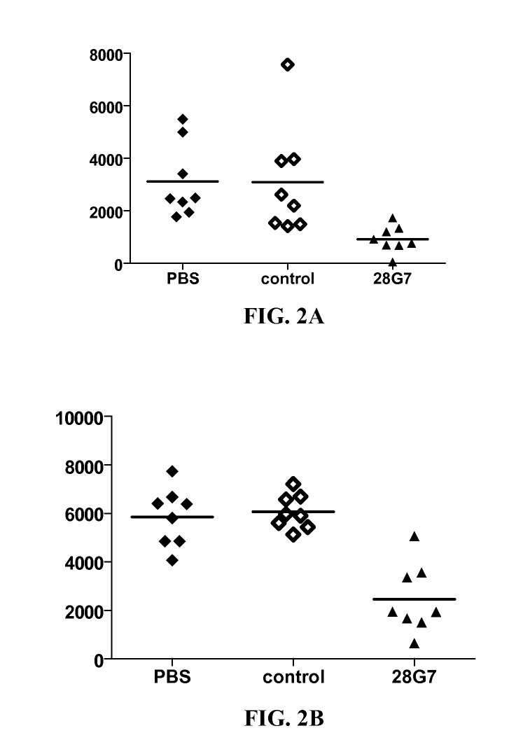

Mouse antibodies were tested for their ability to inhibit TLR3 induced signalling in vivo. Briefly, groups of 5 mice (C57Bl/6J, 8-10 weeks old) are constituted. PBS or anti-TLR3 antibodies (100 μg per mice or 200 μg per mice) is injected IP three hours before polyAU administration IV (20 or 100 μg). Two hours later, blood is withdrawn, serum is prepared and a serum dosage of IL-6 is performed (BD optEIA™ set mIL-6). Results are reported in FIGS. 2A and 2B. In FIG. 2A, is shown the inhibition of 100 μg of anti-mouse TLR3 antibodies 28G7 and a non-functional anti-mouse TLR3 control mAb on IL-6 secretion induced by 20 μg IPH3102. In FIG. 2B, is shown the inhibition of 200 μg of anti-mouse TLR3 antibodies 28G7, a non-functional anti-mouse TLR3 control mAb and a control irrelevant rat IgG pAb, on IL-6 secretion induced by 100 μg IPH3102.

The figures underline the fact that the anti-TLR3 mouse antibodies are able to inhibit a TLR3 ligand (here polyAU) induced signalling.

### Example 4

### Bivalent Affinity

Binding properties of the antibody 28G7 was evaluated using the methods described for SPR, item c). Binding to TLR3 was determined at neutral (pH 7.2) and acid (pH 5.6) conditions, and K_{D} values were calculated. The results (mean of 2 or 3 experiments) are shown in Table 1. At neutral and acid pH, mAb 28G7 showed strong and similar bivalent affinity (K_{D}) for recombinant mouse TLR3 better than 500 picomolar. The affinity of mAb 28G7 was measured in the same conditions in a separate assay, the results are represented in Table 1. These results indicate that the antibody according has a high affinity, especially at acidic pH.

TABLE 1

| | Mean K_{D} (M) at | Mean K_{D} (M) at |
|---|---|---|
| Antibody | pH 7.2 | pH 5.6 |
| 28G7 | 7.05 * 10⁻¹³ | 1.26 * 10⁻¹³ |

### Example 5

### In Vivo Efficacy Model for the Treatment of RA—Preventive Setting

Briefly 20 mice were immunized on day 0 with 100 μg of collagen emulsified in CFA complemented with Mycobater tuberculosis (2 mg/ml) and injected intradermally (ID) at the base of the tail. At day 17, animals were scored (clinical signs often appear prior to the boost), randomized into 2 groups of 8 or 9 mice according to the sum of the 4 limbs clinical score and treated. At days 21, the collagen immunization was boosted by ID administration of collagen alone (100 μg in 50 μl).

The following groups were constituted:
group 1 (PBS, n=9): treated 200 μl twice/week IP
group 2 (28G7, n=8): treated 500 μg/mice twice/week IP

Scoring of the four limbs of the animal was evaluated thrice a week for 3 to 4 weeks. Scoring was evaluated according to table 2.

TABLE 2

| Score | Inflammation level |
|---|---|
| 0 | No evidence of erythema and swelling |
| 1 | Erythema and mild swelling confined to the |
| | tarsals or ankle joint |
| 2 | Erythema and mild swelling extending from the |
| | ankle to the tarsals |
| 3 | Erythema and moderate swelling extending from |
| | the ankle to metatarsal joints |
| 4 | Erythema and severe swelling encompass the ankle, |
| | foot and digits, or ankylosis of the limb |

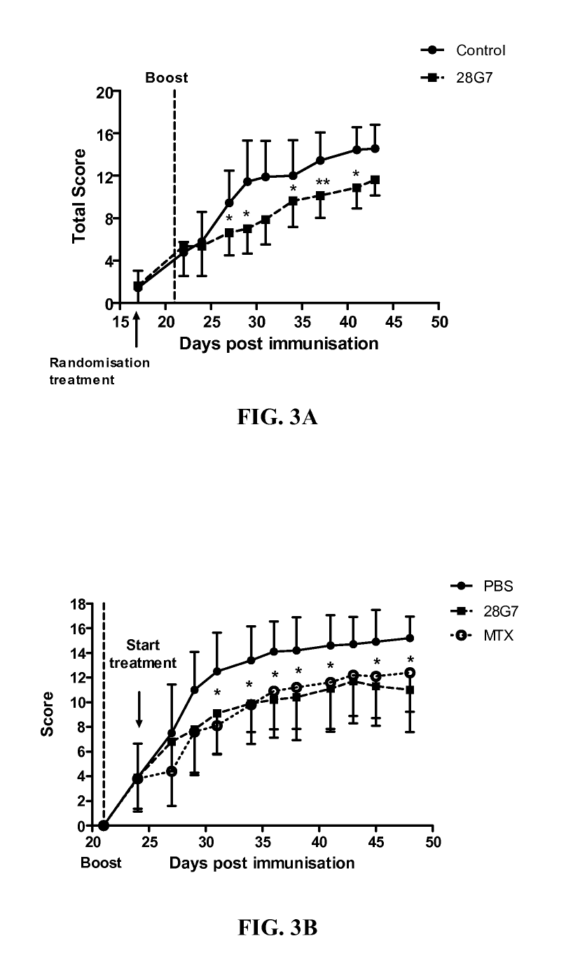

The results are reported in FIG. 3A. The present experiment underlines that the anti-TLR3 antibodies are statistically effective (* p>0.05, ** p>0.005, in Dunnett's test) in the curative treatment of Rheumatoid Arthritis (RA) in comparison with PBS.

### Example 6

### In Vivo Efficacy Model for the Treatment of RA—Curative Setting

Experiment #1: 28G7, PBS, MTX

Briefly 30 mice were immunized on day 0 with intradermal injection of 100 μg of collagen emulsified in CFA complemented with Mycobater tuberculosis (2 mg/ml) at the base of the tail. 21 days later, the collagen immunization is boosted by ID administration of collagen alone (100 μg in 50 μl/mice). At day 24, animals were randomized into 3 groups of 10 mice according to the sum of the 4 limbs clinical score and treatment began.

Group 1 (PBS, n=10): treated 200 μl twice/week IP

Group 2 (Methotrexate—MTX, n=10): treated 2.5 mg/kg twice/week IP

Group 3 (28G7, n=10): treated 500 μg/mice twice/week IP

Scoring of the four limbs of the animal was evaluated thrice a week for 3 to 4 weeks. Scoring was evaluated according to table 2.

The results are reported in FIG. 3B. The present experiment underlines that the anti-TLR3 antibodies are statistically effective in the treatment of an established Rhumatoid Arthritis (RA) in comparison with PBS and Methotrexate (* p>0.05, in Dunnett's test). MTS having a different mechanism of action than the anti-TLR3 antibody, a combination of the two drugs could be beneficial.

Experiment #2: 28G7, PBS, Anti-TNFα Humira™

Briefly 35 mice were immunized on day 0 with intradermal injection of 100 μg of collagen emulsified in CFA complemented with Mycobater tuberculosis (2 mg/ml) at the base of the tail. 21 days later, the collagen immunization is boosted by ID administration of collagen alone (100 μg in 50 μl/mice). At day 24, animals were randomized into 4 groups according to the sum of the 4 limbs clinical score and treatment began.

Group 1 (PBS, n=9): treated 200 μl twice/week IP.

Group 2 (control Ig antibody, n=9): treated 500 μl twice/week IP.

Group 3 (28G7, n=9): treated 500 μg/mice twice/week IP.

Group 4 (Humira™, n=6): treated 100 μl twice/week IP.

Scoring of the four limbs of the animal was evaluated thrice a week for 3 to 4 weeks. Scoring was evaluated according to table 2.

The results are reported in FIG. 3C. The present experiment underlines that the anti-TLR3 antibodies are effective in the treatment of an established Rheumatoid Arthritis (RA) in comparison with PBS and anti-RNFa antibody Humira™. The anti-TNFα having a different mechanism of action than the anti-TLR3 antibody, a combination of the two drugs could be beneficial.

### Example 7

### In Vivo Efficacy Model for the Treatment of Colitis

Four groups of 10 male mice (Balb/c) were used for the model of colitis and one extra group of 8 mice without colitis were used as control (no dosage, intracolonic instillation of saline).

The treated groups were divided as follows:

Group 1: 10 mice received antibody 28G7 (ip, 500 μg/mouse).

Group 2: 10 mice received a non TLR3-relevant antibody administration (ip, 500 μg/mouse).

Group 3: 10 mice received the rat anti-mouse TNF antibody (ip, 15 mg/kg, Humira™).

Group 4: 10 mice received a PBS (ip, 200 μg/mouse).

One hour after in injections, colitis was induced by intracolonic instillation of 2,4,6-trinitrobenzen-sulfonic acid (TNBS) (2 mg/mouse in 40% ethanol of TNBS) in male Balb/C mice (5 to 6 weeks-old). In groups 1, 2 and 3, another injection of either 28G7 or non TLR3-relevant antibody or the anti-TNF antibody was repeated 72 hours after the first antibody injection.

For all groups, several parameters of disease progression were assessed daily: body weight, presence of blood in the feces, presence and severity of diarrhea. All animals were sacrificed for tissue collection 7-days after the induction of colitis. Macroscopic damage score, wall thickness and myeloperoxydase activity (index of granulocyte infiltration), were measured in colonic tissues. Macroscopic damage score is evaluated by observing the parameters as detailed in Table 3.

TABLE 3

| | Score |
|---|---|
| Faecal blood | |
| Diarrhea | |
| Haemorrhage | 0 |
| | 1 (blood in lumen) |
| Adhesion | 0 |
| | 1 (1 point) |
| | 2 (more than 1) |
| | 3 (very severe) |
| Mucus | 0 |
| | 1 |
| Erythema | 0 |
| | 1 (less than 1 cm) |
| | 2 (more than 1 cm) |
| Edema | 0 |
| | 1 (intermediate) |
| | 2 (severe) |
| Ulcer | 0 |
| | 1 (less than 1 cm) |
| | 2 (more than 1 cm) |
| Stricture | 0 |
| | 1 (1 stricture) |
| | 2 (2 stricture) |
| | 3 (more than 2) |
| Total | |

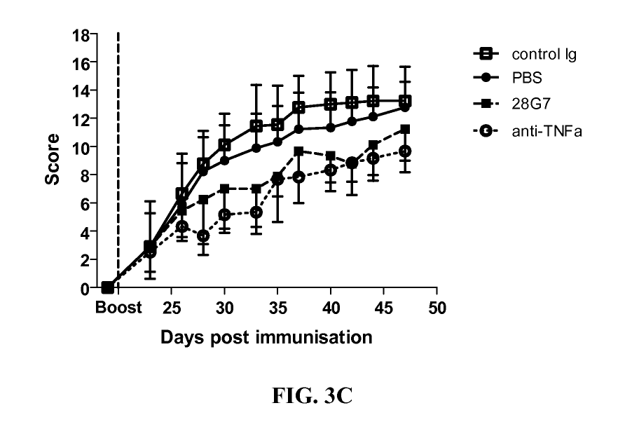

FIG. 4 shows the results of the experiment. FIG. 4A shows the wall thickness measurements for the mice treated with saline (black dots), with TNBS only (black squares) with an anti-TNFα antibody and TNBS (black triangles), with 28G7 and TNBS (open dots), and with a control Ab and TNBS (open squares). FIG. 4B shows the macroscopic damage score for the mice treated with saline (black dots), with TNBS only (black squares) with an anti-TNFα antibody and TNBS (black triangles), with 28G7 and TNBS (open dots), and with a control Ab and TNBS (open squares). These results underline an effect of the anti-TLR3 antibody in the development of the disease, under stringent conditions.

### Example 8

### In Vivo Efficacy Model for the Treatment of Sepsis—CæCal Ligature and Puncture (CLP)—Curative Setting

Briefly 30 mice were operated: the surgery consists in caecal ligature and puncture. By this way the content of the caecal lumen is draining of in the abdominal cavity leading to peritonitis and consequently a septic shock. The CLP is mid-grade, e.g. ligature is performed approximately in the middle of the cecum.

Mice were treated with 28G7 (100 μg/mouse, ip), a control antibody with no TLR3 specificity (“control”, 100 μg/mouse, ip) or the PBS (300 μl/mouse, ip) 6 hours and 24 hours after operation. Survival was assessed at hours 24, 28, 32, 48, 52, 56, 72, 76, 80, 96, 100, 104, 120, 124, 128, 144, 148, 152, 168, 172, 176, 192, 196, 200, 216, 220, 224, 240, 244, 248, 264, 270, 274, 288, 292, 296, 312, 316, 320 and 336. After 336 hours, the mice which have survived have cleared the acute phase infection. The experiment was stopped and mice were sacrificed.

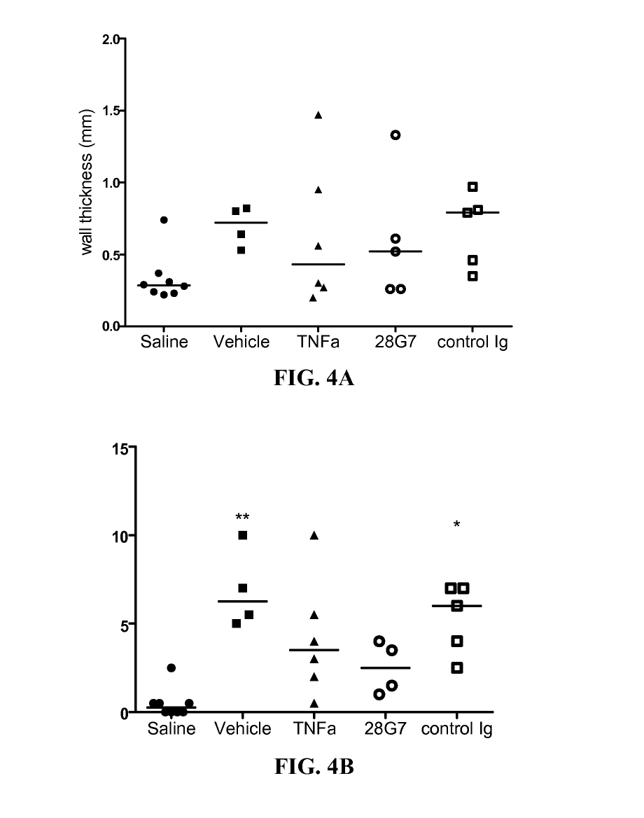

FIG. 5 shows the results of the experiment. The 28G7 treated group has a 80% survival whereas the non-treated group experiences a 40% survival. These results demonstrate that TLR3 antibodies are efficient for the treatment of mice in a CLP mouse model. In this acute model, mice experience an acute infection, mimicking septic shock. The mice which survive have efficiently cleared the acute infection. Therefore, the anti-TLR3 antibodies are suitable agents to treat a patient experiencing a severe acute infection such as a SIRS, a sepsis, a severe sepsis or a septic shock.

### Example 9

### IP-10 Production by Donors in Response to dsRNA and Drug Combinations

IP-10 production was assessed in human donors in response to polyAU (IPH3102), believed to be a specific agonist of TLR3, or plC, believed to be an agonist of TLR3 as well as other dsRNA receptors such as RIG-I and MDA-5. Fresh PBMC were isolated from whole blood of two independent donors. 1.5×10⁶ (donor #1) and 3×10⁶ (donor #2) PBMC per ml were incubated in flat-bottom 96W plates in the presence of 50 μg/ml 31C3 or 34A3 anti-human TLR3 mAbs and a dose range of methotrexate (300, 30 and 3 μg/ml), dexamethasone (200, 20, 2 μg/ml) or Humira® (100, 10, 1 μg/ml). Cells were incubated 1 hr at 37° C. prior addition of 300 μg/ml IPH3102 or 30 μg/ml poly(I:C). Cells were incubated for 24 additional hours at 37° C. Supernatant were then harvested to quantify IP10 production by ELISA. Cells were then recovered, stained with 7-AAD and analyzed by flow cytometry (FACS) to evaluate potential toxic effects of any drugs.

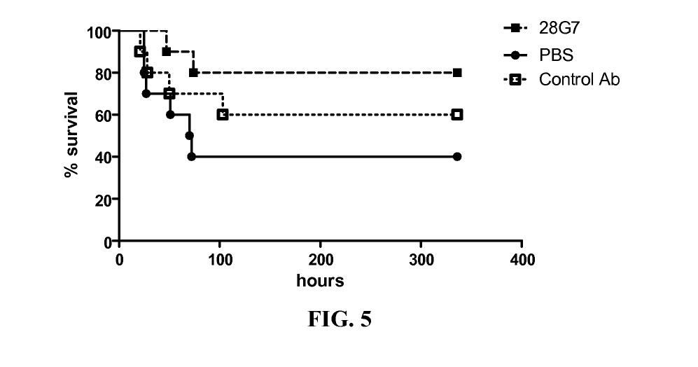

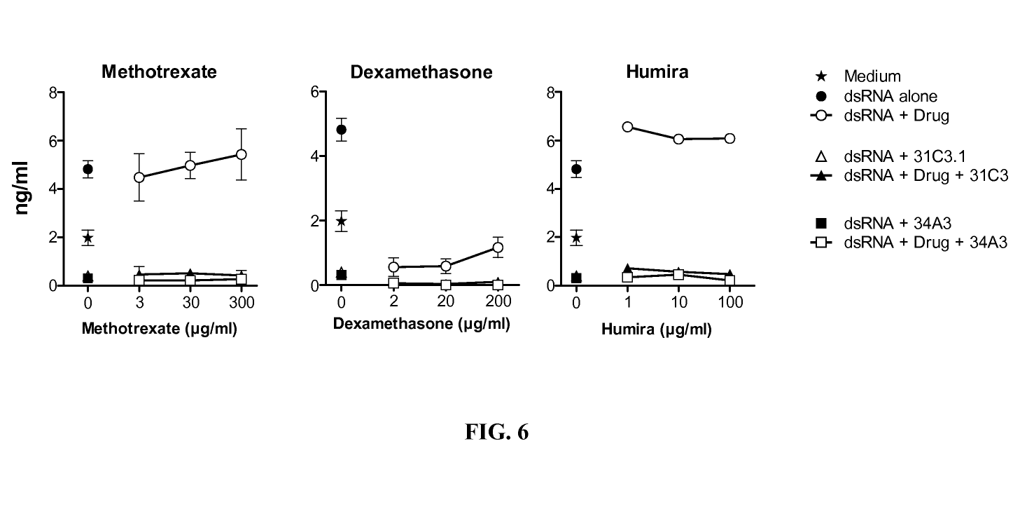

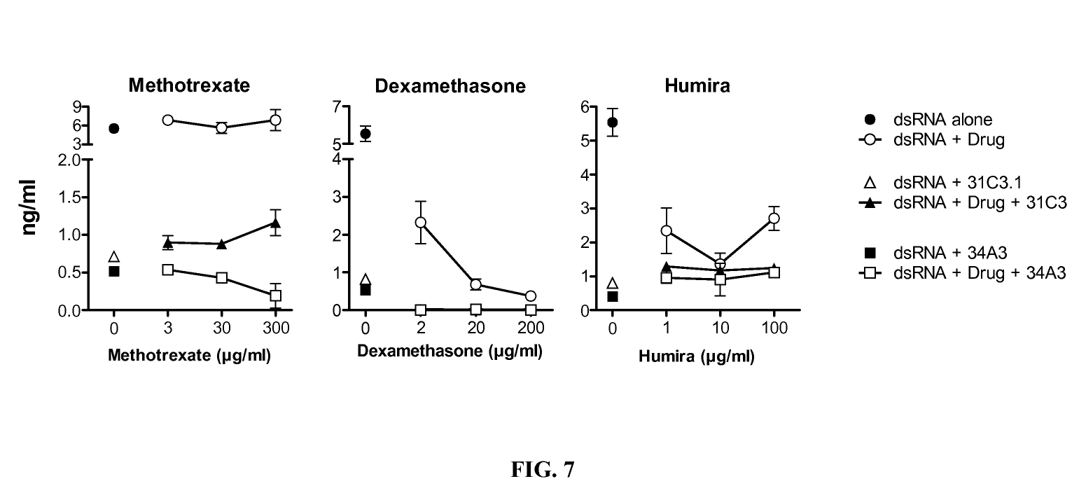

Result of drug combinations with anti-human TLR3 mAbs 31C3 or 34A3 in combination with methotrexate, dexamethasone or Humira® are shown in FIGS. 6, 7 and 8. As shown in FIG. 6 for human donor 2, antibodies 31C3 and 34A3 each substantially reduce IP-10 production in response to polyAU and that the antibodies reduce IP-10 further when combined with methotrexate, dexamethasone or Humira®. In FIG. 6, for each of methotrexate, dexamethasone or Humira®, the data points for antibodies 31C3 and 34A3 (alone or in combination with dsRNA and drug) are superposed. As shown in FIGS. 7 and 24 for human donors 1 and 2 respectively, antibodies 31C3 and 34A3 each substantially reduce IP-10 production in response to polyIC and that the antibodies reduce IP-10 further when combined with methotrexate, dexamethasone or Humira®. In FIG. 7, for dexamethasone the data points for antibodies 31C3 and 34A3 are superposed. In FIG. 8, for each of methotrexate, dexamethasone or Humira, the data points for antibodies 31C3 and 34A3 (alone or in combination with dsRNA and Drug) are superposed, unless indicated otherwise by diverging data points. For human donor 2, IP-10 production in the presence of medium is indicated and IP-10 is shown in intervals of 2 ng/ml because production in the absence of dsRNA was not null. finally, FACS analysis was performed on cells after activation to evaluate the toxic effect of the tested drug combinations. No toxicity was observed.

Anti-TLR3 antibodies can therefore provide an additional effect when used in combination with methotrexate, dexamethasone or Humira. In particular, in response to polyIC the antibodies potentiate the effects of dexamethasone, the treatment of reference in rheumatoid arthritis. Furthermore, the anti-TLR3 antibodies appeared to be more effective that Humira, suggesting that based on IP-10 production, the TNFα pathway is included in the TLR3 response to dsRNA. The anti-TLR3 antibodies can operate by modulating a signaling pathway that is complementary to those modulated by methotrexate, dexamethasone or Humira®, without antagonistic effects, and can therefore be used advantageously in combination with such drugs.

### Example 10

### Anti-TLR3 Antibodies Sensitize Animals to Corticosteroids In Vivo

Corticosteroid treatment is able to protect mice in a model of colitis. For this study a low-dose corticosteroid regimen in which corticosteroids are not sufficiently effective was developed as a model of corticosteroid-insensitivity.

Mild TNBS-Colitis: Six (6) weeks old mice Balb/c were used (Janvier, Le Genest Saint Isle, France). Colitis was induced using the following methodology. On day 0, a catheter fitted to 1 mL syringe was then inserted intracolonically at 2 cm from the anus. To induce colitis, 2 mg of TNBS was administered slowly into the lumen of the colon at a final volume of 0.1 ml/mouse. TNBS (Picrylsulfonic acid solution from Sigma Fluka) was prepared at a final concentration of 20 mg/ml in 30% ethanol (Sigma) in physiological saline and was kept in the dark before use. Ethanol is usually used as a “breaker” of the intestinal barrier, which allows TNBS to penetrate colonic tissues. Control mice received physiological saline (0.1 ml/mouse) using the same technique. All intracolonic administrations were performed under light anaesthesia. Mice were then maintained with the anus held shut for almost 1 minute, to ensure the distribution of TNBS within the bowel and to prevent any leakage. Mice then returned to their home cages. Development of colitis was assessed daily for 7 days beginning on day 0.

All mice were checked every day for the following parameters: Body weight change was calculated and normalized to weight at day 0; Stool consistency (from 0=normal to 2=diarrhea) and detection of blood in stools using a Hemoccult paper test (Hemoccult II papers and revealing solutions from SKD France; score from 0=normal to 2=visible blood) were also evaluated. Both scorings were then pulled to give a daily disease activity index (DAI). Collection of feces was performed during weighting. If no feces were collected, no score was given to the animal. Seven (7) days after TNBS administration, mice were sacrificed by cervical dislocation and laparotomy was performed. The whole colon from anus to caecum was excised, freed of adherent tissue and washed gently to remove any feces. Macroscopic damage scores were also evaluated by observing the following parameters:

| | Score |
|---|---|
| Faecal blood | Cf Hemoccult score |
| Diarrhea | Cf Stool consistency score |
| Haemorrhage | 0 |
| | 1 (blood in lumen) |
| Adhesion | 0 |
| | 1 (1 point) |
| | 2 (more than 1) |
| | 3 (very severe) |
| Mucus | 0 |
| | 1 |
| Erythema | 0 |
| | 1 (less than 1 cm) |
| | 2 (more than 1 cm) |
| Edema | 0 |
| | 1 (medium) |
| | 2 (severe) |
| Ulcer | 0 |
| | 1 (less than 1 cm) |
| | 2 (more than 1 cm) |
| Stricture | 0 |
| | 1 (1 stricture) |
| | 2 (2 strictures) |
| | 3 (more than 2) |
| Total | From 0 to 18 |

Dexamethasone (from Sigma, diluted in sterile NaCl) or vehicle control (NaCl) was administrated by subcutaneous injection (s.c.) 1-hour before TNBS administration and then every day for 6 days with dexamethasone at the dose of 0.1 mg/kg (final volume function of weight) or its vehicle (NaCl, volume function of weight). Anti-mouse TLR3 or Isotype Control rat IgG1 Ab (diluted in PBS) was administrated by intraperitoneal injection (i.p) 1-hour before TNBS administration and then repeated at day 3 with anti-moTLR3 or isotype control rat IgG1 at the dose of 500 μg/mouse in 200 μl. Statistical analysis was performed using GraphPad Prism® (Version 5; GraphPad Software Inc., La Jolla, Calif., USA). A p<0.05 was accepted for statistical significance.

### Results:

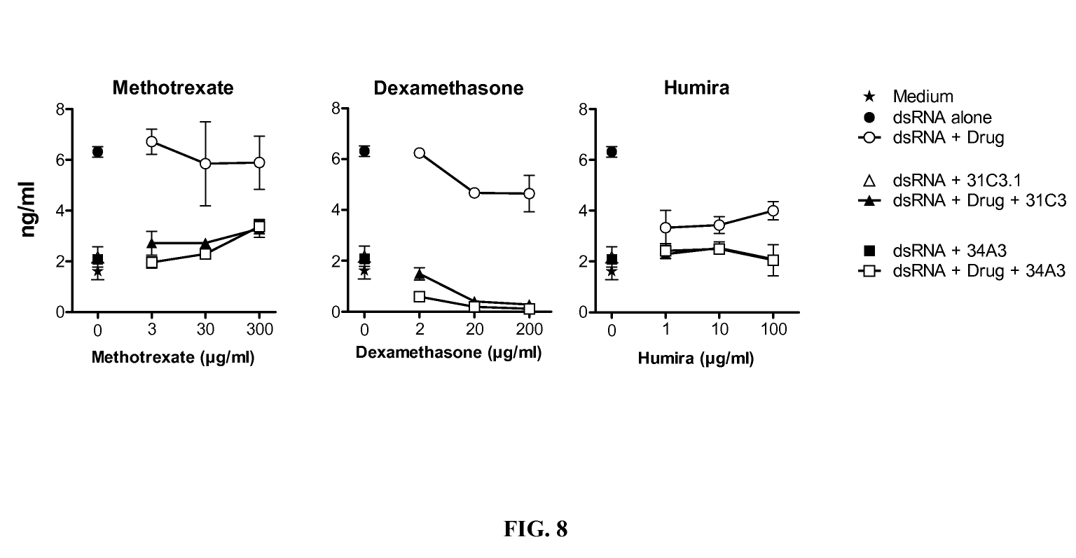

While TNBS at a dose of 2 mg/mouse in 30% ethanol increased significantly weight loss from day 4 to 7, compared to control (saline) group, in animals that have received the Isotype Control rat IgG1 Ab, in mice that have received the anti-moTLR3 antibody, TNBS did not induce any significant weight loss. This suggests that the treatment with the anti-TLR3 antibody is able to inhibit TNBS-induced weight loss (FIG. 9). Dexamethasone treatment at a dose of 0.5 mg/kg could not inhibit weight loss induced by TNBS instillation in mice that have also received a treatment with the control antibody. On the contrary, dexamethasone treatment seemed to exacerbate the weight loss induced by TNBS: the significance compared to control (saline) group were higher in mice treated with dexamethasone, than in mice treated with the vehicle of dexamethasone (FIG. 9). Combo treatment with both dexamethasone and anti-moTLR3 antibody significantly inhibited weight loss: the weights of those mice were not different from control non-inflamed mice, except at day 7 (FIG. 9). Here again, treatment with the anti-moTLR3 antibody seemed to protect mice from weight loss upon colitis.

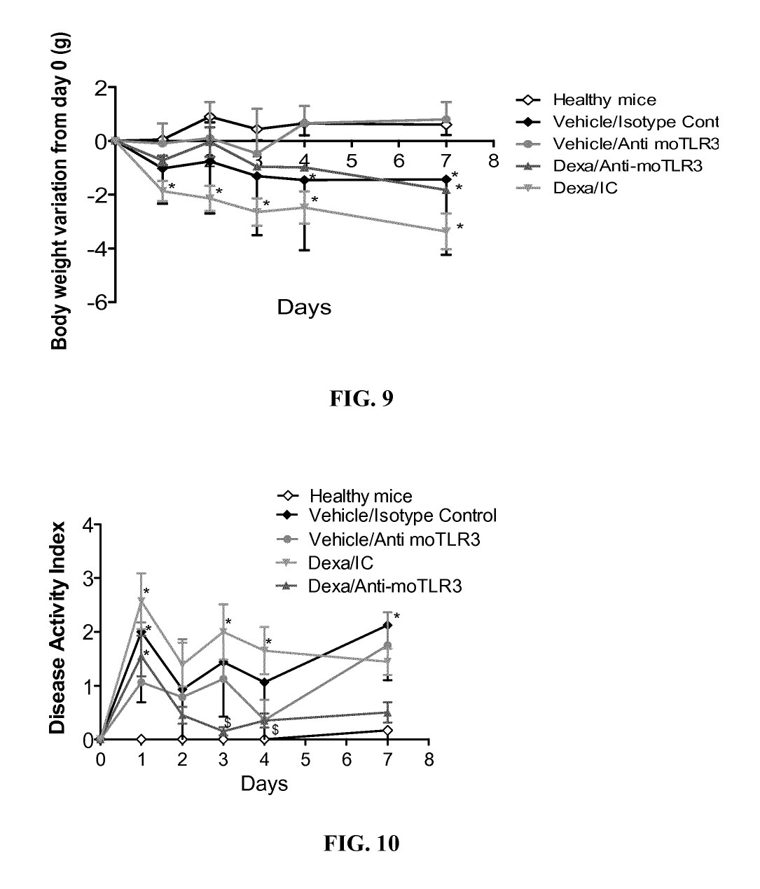

In mice treated with the anti-moTLR3 antibody, no significant increase in disease activity over control non-inflamed mice (receiving saline) was observed (FIG. 10), as opposed to mice receiving the isotype control rat IgG1 Ab, which showed significant increased disease activity compared to controls (FIG. 10). This suggests that the treatment with anti-moTLR3 antibody, but not treatment with an irrelevant antibody, is able to inhibit TNBS-induced disease activity (FIG. 10B). Dexamethasone treatment did not inhibit TNBS-induced disease activity in mice that have received the isotype control rat IgG1 Ab (FIG. 10), this activity was significantly elevated at days 1, 3 and 4 after inducing colitis. However, combo treatment with both dexamethasone and the anti-moTLR3 antibody significantly inhibited disease activity, which was comparable at that observed in control non-inflamed (saline) group, from day 2 to day 7 (FIG. 10B). Here again, Anti-moTLR3 treatment reduced signs of colitis.

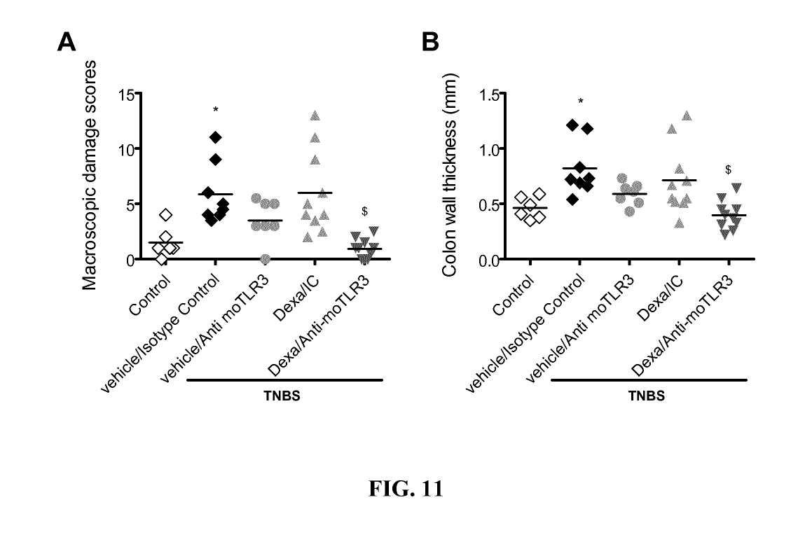

Administration of TNBS increased significantly macroscopic damage scores compared to saline mice in the group treated with the vehicle of dexamethasone and with the isotype control rat IgG1 Ab (FIG. 11A). After administration of TNBS, the wall thickness was also significantly increased compared to saline-treated group in mice treated with the vehicle of dexamethasone and with the isotype control rat IgG1 Ab (FIG. 11B) Treatment with the anti-moTLR3 antibody reduced tissue damage associated with TNBS-induced colitis and observed 7-days after TNBS, both alone or in combo treatment with dexamethasone (FIG. 11A): no significant increased damage score was observed compared to controls, in groups that have received the anti-moTLR3 treatment (FIG. 11A). Similarly, colonic wall thickness was significantly increased 7-days post TNBS enema in mice treated with the isotype control rat IgG1 Ab, but not in mice treated with the anti-moTLR3 antibody (FIG. 11A). This demonstrates that treatment with only the anti-moTLR3 antibody is able to reduce TNBS-induced increased wall thickness.

In mice that have received a dexamethasone treatment (0.5 mg/kg), the damage scores observed in tissues 7-days after the induction of colitis by TNBS enema were not significantly increased compared to control non-inflamed mice (FIG. 11). While dexamethasone treatment is known to have protective effects at higher doses, at the 0.5 mg/kg dexamethasone had little protective effect, at a level close to that of the isotype control rat IgG1 Ab, as observed by the lack of significant decrease in damage score of those groups, compared to controls. However, animals that received the combination treatment with dexamethasone and anti-moTLR3 treatment had almost no damage to their tissues and were fully protected compared to mice that had the combination treatment with dexamethasone and the isotype control rat IgG1 Ab (FIG. 11A). Damage score was significantly lower in dexamethasone- and 28G7-treated group compared to dexamethasone-isotype control-treated group (FIG. 3A, p<0.05).

Colonic wall thickness was significantly increased by TNBS in mice that have received treatment with the isotype control rat IgG1 Ab, (FIG. 11B), and this significant increase was also observed in mice treated with dexamethasone (FIG. 11B). However, the mice that had received the combo treatment dexamethasone+anti TLR3 had a significantly lower wall thickness compared to mice that had received dexamethasone+isotype control (FIG. 11B, p<0.05). This suggests that the combo treatment with dexamethasone plus the anti-moTLR3 antibody protects against mucosal in a corticosteroid-insensitive model.

### Example 11

### Anti-TLR3 Antibodies Sensitize Human Epithelial Bronchial Cells to Corticosteroids

IL-6 and IP-10 production was assessed in PBMC and virus-transformed lung epithelial BEAS-2B cell line in response to high molecular weight polyAU (IPH3102), a specific agonist of TLR3. Experiments with BEAS-2B were performed in Bronchial Epithelial Growth Medium (Lonza, #CC-3170). 2×10⁴ BEAS-2B per well were plated in flat-bottom 96W plates in 100 μl culture medium and incubated over night at 37° C. A part of the cells was treated for 24-supplemental hours with 1000 UI/ml Intron A (Schering Plough, IFN-alpha 2b). Medium was discarded and 37B7 anti-human TLR3 Ab (0.01 and 0.1 μg/ml in the condition without Intron A and 0.01 and 0.3 μg/ml after treatment with Intron A) was added to the culture. Cells were incubated 30 min at 37° C. prior addition of Dexamethasone (1, 0.1, 0.01, 0.001 μg/ml in the condition with Intron A and 10, 1, 0.1, 0.01 μg/ml after treatment with Intron A) or Budesonide (1, 0.1, 0.01, 0.001 μg/ml). Cells were incubated 30-additional min at 37° C. before addition of 300 μg/ml IPH3102. Cells were finally incubated for 24-additional hours and supernatants were harvested to quantify IL-6 and IP-10 production by ELISA.

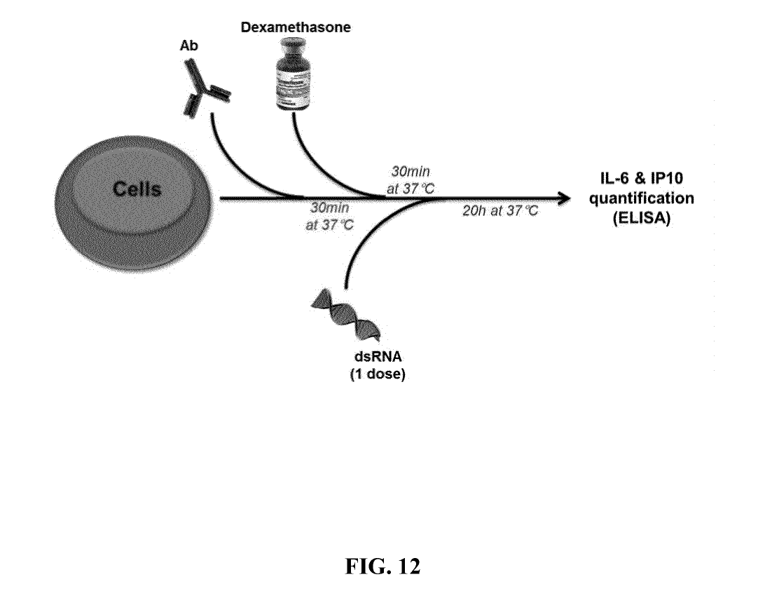

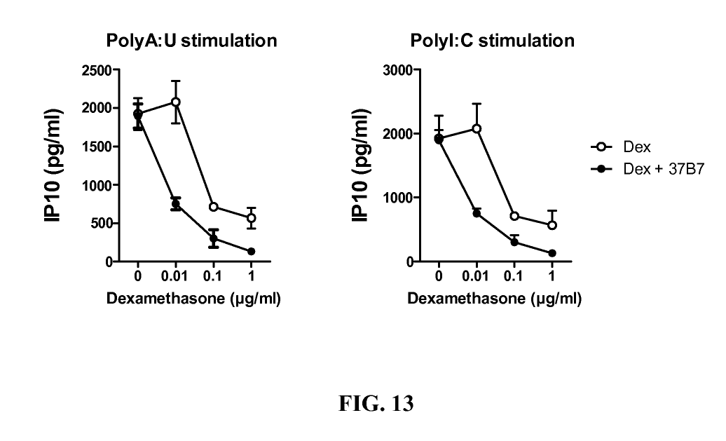

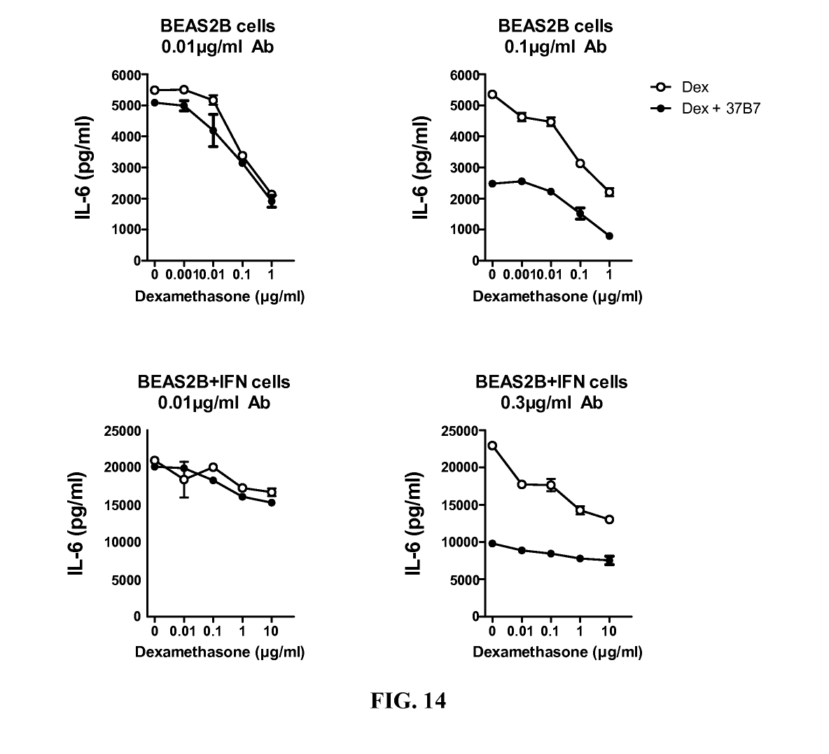

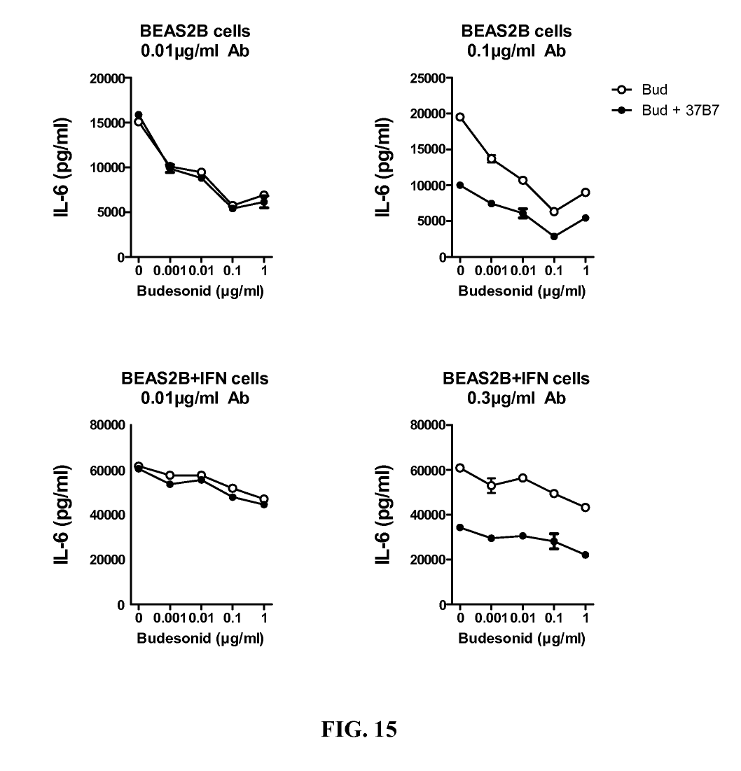

The experimental setup is summarized in FIG. 12. Results with PBMC are shown in FIG. 13. It can be seen that anti-TLR3 antibodies sensitize human PBMC to corticosteroids, illustrated by reduction in IP-10 production in response to double stranded RNA. Results with BEAS-2B cells are shown in FIGS. 14 and 15. It can be seen that anti-TLR3 antibodies sensitize human epithelial bronchial cells to corticosteroids, illustrated by reduction in IP-10 (FIG. 14) and IL-6 (FIG. 15) production in response to double stranded RNA in a virus-transformed lung epithelial BEAS-2B cell line model.

As expected, only the highest concentration of anti-TLR3 Ab blocks almost 50% of the BEAS-2B IL-6 production. Likewise, both Dexamethasone and Budesonide inhibit IL-6 production in a dose-dependent manner. A strong additive inhibition of IL-6 production was observed when the highest anti-TLR3 Ab dose was combined with either dexamthesone or Budesonid. Thus, combination treatment will allow to strongly enhance anti-inflammatory properties of corticosteroids used alone.

While sensitization to corticosteroids is observed in both the PBMC and the bronchial epithelial cell models, the cooperative effects of anti-TLR3 antibody and corticosteroids appears to be particularly synergistic in a complex inflammatory model (such as PBMC) involving cell-to-cell contact and cross-talk among different cell populations such as DC (on which anti-TLR3 may have a strong modulatory effect), B cells and/or T cells, compared to the single population BEAS-2B model. The BEAS-2B model may more closely model a disease such as COPD wheareas the PBMC model may resemble a more complex immune-mediated (e.g. B cell-, T cell-) driven disease.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents, The description herein of any aspect or embodiment of the invention using terms such as reference to an element or elements is intended to provide support for a similar aspect or embodiment of the invention that “consists of′,” “consists essentially of” or “substantially comprises” that particular element or elements, unless otherwise stated or clearly contradicted by context (e. g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-38. (canceled)

39. A method for treating an autoimmune or inflammatory disease in an individual comprising administering to the individual an anti-TLR3 agent that inhibits TLR3 signaling in combination with a corticosteroid at a dose of 10 mg per day or less of prednisone equivalent.

40. The method of claim 39, wherein the subject has corticosteroid insensitivity.

41. The method of claim 39, wherein the corticosteroid is administered at a dose of 1 mg/kg per day or less than 1 mg/kg per day of prednisone equivalent.

42. The method of claim 39, wherein the anti-TLR3 agent and the corticosteroid are administered separately.

43. The method of claim 39, wherein the method comprises: (a) evaluating whether said patient has corticosteroid insensitivity; and (b) if said patient has corticosteroid insensitivity, administering to said patient an effective dose of an anti-TLR3 agent in combination with a corticosteroid.

44. The method of claim 39, wherein the corticosteroid is administered at a dose of 1 mg/kg per day or less of beclomethasone equivalent.

45. The method of claim 39, wherein the subject has a disorder selected from the group consisting of asthma, corticosteroid-resistant asthma, severe asthma, COPD, and sarcoidosis.

46. The method of claim 39, wherein the anti-TLR3 agent is an anti-TLR3 antibody that binds to a TLR3 polypeptide and inhibits dsRNA-induced signaling by a TLR3 polypeptide.

47. The method of claim 41, wherein the corticosteroid is administered at least 3 times per week by oral, injection or infusion route, wherein the anti-TLR3 antibody is administered by injection or infusion in an amount between about 0.01 and 20 mg/kg, at a frequency of from about once per week to about once every 2 months.

48. The method of claim 47, wherein the corticosteroid is administered at a dose of 40 mg per day or less than 40 mg per day of prednisone equivalent and the subject has systemic lupus erythematosus (SLE).

49. The method of claim 39, wherein the corticosteroid is administered at least 3 times per week by oral, injection or infusion route, wherein the anti-TLR3 antibody is administered by injection or infusion in an amount between about 0.01 and 20 mg/kg, at a frequency of from about once per week to about once every 2 months, and wherein the subject has lupus, asthma, corticosteroid-resistant asthma, severe asthma, COPD, sarcoidosis, sepsis or acute or chronic Graft versus Host Disease.

50. The method of claim 41, wherein the corticosteroid is administered at least 3 times per week by oral, injection or infusion route, wherein the anti-TLR3 antibody is administered by injection or infusion in an amount between about 0.01 and 20 mg/kg, at a frequency of from about once per week to about once every 2 months, and wherein the subject has lupus, asthma, corticosteroid-resistant asthma, severe asthma, COPD, sarcoidosis, sepsis or acute or chronic Graft versus Host Disease.

51. The method of claim 39, wherein the corticosteroid is selected from the group consisting of hydrocortisone (Cortisol), cortisone acetate, prednisone, prednisolone, methylprednisolone, deflazacort, betamethasone, triamcinolone, beclometasone, paramethasone, fluticasone, fludrocortisone acetate, deoxycorticosterone acetate (DOCA), fluprednisolone, fluticasone propionate, budesonide, beclomethasone dipropionate, flunisolide and triamcinolone acetonide.

52. The method of claim 39, wherein the anti-TLR3 agent is a monoclonal antibody that specifically binds and inhibits the signaling of a TLR3 polypeptide, wherein said antibody inhibits signaling by the TLR3 polypeptide, and has a Kd of less than 10⁻⁹M for binding to a TLR3 polypeptide at both neutral and acidic pH.

53. A method for treating an autoimmune or inflammatory disease in an individual having corticosteroid insensitivity comprising administering to the individual an anti-TLR3 agent that inhibits TLR3 signaling.

54. A method for the treatment of an autoimmune or inflammatory disease in an individual, for determining an optimal treatment regimen, or for selecting an individual for inclusion in a clinical trial, the method comprising:
(a) evaluating whether an individual is corticosteroid insensitive; and
(b) if said individual is corticosteroid insensitive, administering to said patient an effective dose of an anti-TLR3 agent that inhibits TLR3 signaling, in combination with a corticosteroid.

55. The method of claim 54, wherein the step of evaluating whether an individual is corticosteroid insensitive comprises obtaining a biological sample from the individual and determining in vitro the presence of one or more biomarkers of corticosteroid insensitivity, optionally wherein the biomarker is an inflammatory mediator and/or markers of ongoing inflammation or disease.

56. The method of claim 54, wherein the corticosteroid is administered at a dose of 1 mg/kg per day or less than 1 mg/kg per day of prednisone equivalent.

57. The method of claim 54, wherein the corticosteroid is administered at a dose of 10 mg per day or less of prednisone equivalent.


SEQUENCE LISTING (80 sequences)

SEQ ID NO: 1 - protein, 904 aa, HOMO SAPIENS
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 2 - protein, 5 aa, Rattus norvegicus
NYWMN

SEQ ID NO: 3 - protein, 6 aa, Rattus norvegicus
GYTFTN

SEQ ID NO: 4 - protein, 10 aa, Rattus norvegicus
GYTFTNYWMN

SEQ ID NO: 5 - protein, 17 aa, Rattus norvegicus
MIDPSDSETHYNQMFKD

SEQ ID NO: 6 - protein, 10 aa, Rattus norvegicus
MIDPSDSETH

SEQ ID NO: 7 - protein, 11 aa, Rattus norvegicus
GASSDYYYFDY

SEQ ID NO: 8 - protein, 11 aa, Rattus norvegicus
ITSTDIHDDIN

SEQ ID NO: 9 - protein, 7 aa, Rattus norvegicus
EGNTLRP

SEQ ID NO: 10 - protein, 9 aa, Rattus norvegicus
LQSDNLPRT

SEQ ID NO: 11 - protein, 5 aa, Rattus norvegicus
YTYMH

SEQ ID NO: 12 - protein, 6 aa, Rattus norvegicus
GYNIRY

SEQ ID NO: 13 - protein, 10 aa, Rattus norvegicus
GYNIRYTYMH

SEQ ID NO: 14 - protein, 17 aa, Rattus norvegicus
RIDPANGDSIYGEDFKT

SEQ ID NO: 15 - protein, 10 aa, Rattus norvegicus
RIDPANGDSI

SEQ ID NO: 16 - protein, 8 aa, Rattus norvegicus
GEFDYFDY

SEQ ID NO: 17 - protein, 11 aa, Rattus norvegicus
LASEGISNDLA

SEQ ID NO: 18 - protein, 7 aa, Rattus norvegicus
AASRLQD

SEQ ID NO: 19 - protein, 9 aa, Rattus norvegicus
QQNYKYPLT

SEQ ID NO: 20 - protein, 5 aa, Rattus norvegicus
STYIY

SEQ ID NO: 21 - protein, 6 aa, Rattus norvegicus
GYNIWS

SEQ ID NO: 22 - protein, 10 aa, Rattus norvegicus
GYNIWSTYIY

SEQ ID NO: 23 - protein, 17 aa, Rattus norvegicus
RIDPANGNTIYAEKFKT

SEQ ID NO: 24 - protein, 10 aa, Rattus norvegicus
RIDPANGNTI

SEQ ID NO: 25 - protein, 10 aa, Rattus norvegicus
GDHGGYVMDA

SEQ ID NO: 26 - protein, 11 aa, Rattus norvegicus
LASEDIYSYLA

SEQ ID NO: 27 - protein, 7 aa, Rattus norvegicus
AANRLED

SEQ ID NO: 28 - protein, 9 aa, Rattus norvegicus
LQGSEFPYT

SEQ ID NO: 29 - protein, 5 aa, Rattus norvegicus
SNYMH

SEQ ID NO: 30 - protein, 6 aa, Rattus norvegicus
GYTFTS

SEQ ID NO: 31 - protein, 10 aa, Rattus norvegicus
GYTFTSNYMH

SEQ ID NO: 32 - protein, 17 aa, Rattus norvegicus
WIFPGDGDTNYNQKFNG

SEQ ID NO: 33 - protein, 10 aa, Rattus norvegicus
WIFPGDGDTN

SEQ ID NO: 34 - protein, 9 aa, Rattus norvegicus
SDGDWYFDF

SEQ ID NO: 35 - protein, 11 aa, Rattus norvegicus
QASEDIYSGLA

SEQ ID NO: 36 - protein, 7 aa, Rattus norvegicus
AASRLQD

SEQ ID NO: 37 - protein, 9 aa, Rattus norvegicus
QQGVKYPNT

SEQ ID NO: 38 - protein, 5 aa, Rattus norvegicus
YTYMH

SEQ ID NO: 39 - protein, 6 aa, Rattus norvegicus
GYNITY

SEQ ID NO: 40 - protein, 10 aa, Rattus norvegicus
GYNITYTYMH

SEQ ID NO: 41 - protein, 17 aa, Rattus norvegicus
RIDPANGNTIYGEKFKN

SEQ ID NO: 42 - protein, 10 aa, Rattus norvegicus
RIDPANGNTI

SEQ ID NO: 43 - protein, 8 aa, Rattus norvegicus
GEFDYFDH

SEQ ID NO: 44 - protein, 11 aa, Rattus norvegicus
LASEDISNDLA

SEQ ID NO: 45 - protein, 7 aa, Rattus norvegicus
AASRLED

SEQ ID NO: 46 - protein, 9 aa, Rattus norvegicus
QQSYKYPVT

SEQ ID NO: 47 - protein, 5 aa, Mus musculus
AYYMH

SEQ ID NO: 48 - protein, 6 aa, Mus musculus
GYSFTA

SEQ ID NO: 49 - protein, 10 aa, Mus musculus
GYSFTAYYMH

SEQ ID NO: 50 - protein, 17 aa, Mus musculus
RINPYNGATSYNRNFKD

SEQ ID NO: 51 - protein, 10 aa, Mus musculus
RINPYNGATS

SEQ ID NO: 52 - protein, 9 aa, Mus musculus
SGGNTYFDY

SEQ ID NO: 53 - protein, 10 aa, Mus musculus
RASENIYSSL

SEQ ID NO: 54 - protein, 7 aa, Mus musculus
NAKTLAE

SEQ ID NO: 55 - protein, 9 aa, Mus musculus
QHHYGTPPT

SEQ ID NO: 56 - protein, 5 aa, Mus musculus
TYSIY

SEQ ID NO: 57 - protein, 6 aa, Mus musculus
GYVFTT

SEQ ID NO: 58 - protein, 10 aa, Mus musculus
GYVFTTYSIY

SEQ ID NO: 59 - protein, 17 aa, Mus musculus
YIDPYNGDTSYNQKFKG

SEQ ID NO: 60 - protein, 10 aa, Mus musculus
YIDPYNGDTS

SEQ ID NO: 61 - protein, 10 aa, Mus musculus
EGNYYGYFDY

SEQ ID NO: 62 - protein, 10 aa, Mus musculus
SASSSVSYMF

SEQ ID NO: 63 - protein, 6 aa, Mus musculus
LTSNLA

SEQ ID NO: 64 - protein, 9 aa, Mus musculus
QQWTGNPPT

SEQ ID NO: 65 - protein, 125 aa, Rattus norvegicus
QVQLQQPGAELVRPGASVKLSCKASGYTFTNYWMNWVKQRPGQGLEWIGMIDPSDSETHYNQMFKDKATLTVDKSSSTAYMQLSSLTSEDSAVYYCARGGASSDYYYFDYWGQGTTLTVSSASTK

SEQ ID NO: 66 - protein, 109 aa, Rattus norvegicus
ETTVTQSPASLSMAIGEKVTIRCITSTDIHDDINWYQQKPGEPPKLLISEGNTLRPGVPSRFSSSGYGTDFVFTIENMLSEDVADYYCLQSDNLPRTFGGGTKLEIKRT

SEQ ID NO: 67 - protein, 121 aa, Rattus norvegicus
EVQLQQSGAELGKPGTSVKLSCKVSGYNIRYTYMHWVNQRPGKGLEWIGRIDPANGDSIYGEDFKTKATLTADTSSNTAYMQLSQLKSDDTAIYFCAMGEFDYFDYWGQGVMVTVSSASTK

SEQ ID NO: 68 - protein, 107 aa, Rattus norvegicus
DIQMTQSPPSLSASLGETVSIECLASEGISNDLAWYQQRSGKSPQLLIYAASRLQDGVPSRFSGSGSGTRYSLKISGMQPEDEADYFCQQNYKYPLTFGSGTKLEIK

SEQ ID NO: 69 - protein, 119 aa, Rattus norvegicus
EVQLQQYGAELGKPGTSVKLSCKVSGYNIWSTYIYWVNQRPGKGLEWIGRIDPANGNTIYAEKFKTKATLTADTSSNTAYMQLSQLKSDDTAIYFCAMGDHGGYVMDAWGQGASVTVSS

SEQ ID NO: 70 - protein, 107 aa, Rattus norvegicus
DIQMTQSPGSLSASLGETVSIECLASEDIYSYLAWYQQKPGKSPQLLIYAANRLEDGVPSRFSGSGSGTQYSLKISGMQPEDEGDYFCLQGSEFPYTFGTGTKLELK

SEQ ID NO: 71 - protein, 118 aa, Rattus norvegicus
QVQLQQSGTELVKPGSSVKISCKASGYTFTSNYMHWIRQLPGNGLEWIGWIFPGDGDTNYNQKFNGKATLTADKSSSTAYMQLSSLTSEDYAVYFCARSDGDWYFDFWGPGTMVTVSS

SEQ ID NO: 72 - protein, 107 aa, Rattus norvegicus
DIQMTQSPGSLSASLGETVTIQCQASEDIYSGLAWYQQKPRKSPQLLISAASRLQDGVPSRFSGSGSGTQYSLKISSMQTEDEGVYFCQQGVKYPNTFGPGTKLELK

SEQ ID NO: 73 - protein, 117 aa, Rattus norvegicus
EVQLQQYGAELGKPGTSVKLSCKVSGYNITYTYMHWVNQRPGKGLEWIGRIDPANGNTIYGEKFKNKATLTADTSSNTAYMQLSQLKSDDTAIYFCAMGEFDYFDHWGQGVMVTVSS

SEQ ID NO: 74 - protein, 107 aa, Rattus norvegicus
DIQMTQSPASLSASLGETVSIECLASEDISNDLAWYQQKSGKSPQVLIYAASRLEDGVPSRFSGSGSGTRYSLKISGMQPEDEADYFCQQSYKYPVTFGSGTKLEIK

SEQ ID NO: 75 - protein, 136 aa, Mus musculus
MGWSWIFLFLLSGTAGVLSEVQLQQSGPELVKPGASVKISCKPSGYSFTAYYMHWVKQSHVKSLEWIGRINPYNGATSYNRNFKDKASLTVDKSSSTAYMELHSLTSEDSAVYYCARSGGNTYFDYWGQGTTLTVS

SEQ ID NO: 76 - protein, 128 aa, Mus musculus
MSVPTQVLGLLLLWLTGARCDIQMTQSPASLSASVGETVTITCRASENIYSSLAWYQQKQGKSPQLLVYNAKTLAEGVPSRFSGSGSGTQFSSLKINSLQPEDFGTYYCQHHYGTPPTFGGGTKLEIK

SEQ ID NO: 77 - protein, 137 aa, Mus musculus
MEWRWIFLFLLSGTTGVHSEIQLQQSGPELVKPGASVKVSCKASGYVFTTYSIYWVKQSHGKSLEWIGYIDPYNGDTSYNQKFKGKATLTVDKSSSTAYMHLNSLTSEDSTVYYCAREGNYYGYFDYWGQGTTLTVS

SEQ ID NO: 78 - protein, 128 aa, Mus musculus
MDFQVQIFSFLLMSASVIMSRGQIVLTQSPALMSASPGEKVTMTCSASSSVSYMFWYQQKPRSSPKPWIYLTSNLASGVPARFSGSGSGTSYSLTISSMEAEDAATYYCQQWTGNPPTFGGGTKLEIK

SEQ ID NO: 79 - protein, 448 aa, HOMO SAPIENS
EVQLVQSGAEVKKPGESLKISCKGSGYSFTNYWVGWVRQMPGKGLEWMGFIDPSDSYTNYAPSFQGQVTISADKSISTAYLQWSSLKASDTAMYYCARELYQGYMDTFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 214 aa, HOMO SAPIENS
DIQMTQSPSSLSASVGDRVTITCRASQSIGLYLAWYQQKPGKAPKLLIYAASSLQSGVPSRFSGSGSGTDFTLTISSLQPEDFATYYCQQGNTLSYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC